(19) United States
(12) Patent Application Publication
(10) Pub. No.: US 2007/0167526 A1
(43) Pub. Date: Jul. 19, 2007

(54) Topical mecamylamine formulations for ocular administration and uses thereof

(76) Inventors: Xiaoming Zhang, Sunnyvale, CA (US); Muralitharan Kengatharan, Menlo Park, CA (US); John P. Cooke, Palo Alto, CA (US); Harun Takruri, Newport Beach, CA (US)

Correspondence Address: MORRISON & FOERSTER LLP, 755 PAGE MILL RD, PALO ALTO, CA, 94304-1018, US

(21) Appl. No.: 11/641,192

(22) Filed: Dec. 18, 2006

Related U.S. Application Data
(60) Provisional application No. 60/859,582, filed on Nov. 17, 2006.
(60) Provisional application No. 60/838,605, filed on Aug. 17, 2006.
(60) Provisional application No. 60/751,808, filed on Dec. 19, 2005.

(51) Int. Cl. A61K 31/137

(52) U.S. Cl. 514/649

(57) ABSTRACT

Provided are methods, pharmaceutical formulations and kits thereof for the treatment and/or prevention of conditions mediated by neovascularization, abnormal angiogenesis, vascular permeability, or combinations thereof, of posterior and/or anterior tissues and fluids of the eye, including conditions associated with proliferative retinopathies, for example, diabetic retinopathy, age-related maculopathy, retinopathy of prematurity, retinopathy associated with macular edema, or retinopathy associated with sickle cell disease, using the topical administration of mecamylamine or a pharmaceutically acceptable salt thereof to the eye. Methods of preparing the pharmaceutical formulations are also provided.

### CROSS REFERENCE TO RELATED APPLICATIONS

This application claims benefit of U.S. Provisional Application No. 60/859,582, filed on Nov. 17, 2006, Provisional Application No. 60/838,605, filed on Aug. 17, 2006 and Provisional Application No. 60/751,808, filed on Dec. 19, 2005, the disclosures of which are incorporated herein by reference in their entirety.

### BACKGROUND OF THE INVENTION

Age related macular degeneration (AMD) is the leading cause of irreversible severe vision loss among the elderly in North America and Europe (See Arch Ophthalmol. (2004), 1122: 564-72; Olejnik et al., (2005) Adv. Drug. Dev. Rev. 57: 1991-1993; Kulkarni et al., (2005) Adv. Drug. Dev. Rev. 57: 1994-2009; Gryziewicz (2005) Adv. Drug. Dev. Rev. 57: 2092-2098). There are two forms of AMD: The non-neovascular (also known as dry form or non-exudative) and the neovascular (also known as wet form or exudative). Though less common, the neovascular form accounts for the majority of cases of blindness. In the neovascular AMD, newly created abnormal blood vessels grow under the center of the retina, a process called choroidal neovascularization, which is then accompanied by vascular leakage and permeability. This leads to scarring of the retina resulting in distorted vision or destruction of central vision. While several growth factors are associated with the angiogenic processes, the isozymes of VEGF (vascular endothelial growth factor) are central to the sequence of events, leading to neovascularization, angiogenesis and vascular leakage (See Ferrara et al. Recent Prog Horm Res., (2000), 55: 15-35, discussion 35-6). The dry or non-neovascular form of macular degeneration often appears prior to the diagnosis of the neovascular (“wet”) form of macular degeneration and is a risk factor for the development of the neovascular form of macular degeneration. Individuals at higher risk for developing the neovascular form of macular degeneration are those with the large areas of macular degeneration caused by the non-neovascular form.

Animal studies have shown that VEGF expression is sufficient to induce neovascularization in the eye (see e.g., Tolentino et al., Arch Ophthalmol. 1996, 1114:964-70), whereas its antagonists reduce or eliminate this effect (see e.g. Adamis et. al., Arch Ophthalmol., 1996, 1114:66-71). Moreover, the presence of VEGF is temporally and spatially correlated with ocular neovascularization in the primate model (see e.g., Miller et al. Am J. Pathol., 1994, 145: 574-84). Patients with ocular neovascularization secondary to proliferative diabetic retinopathy also have elevated vitreous levels of VEGF (see e.g., Aiello et al. N Engl J Med., 1994, 331: 1480-7).

Currently available modalities for the treatment of exudative AMD include thermal laser photocoagulation, photodynamic therapy and administration of the VEGF receptor antagonist pegaptanib (Macugen®; Pfizer) by intraocular injections, which are invasive and can cause significant side effects, such as retinal detachment, vitreous hemorrhage, endophthalmitis, and lens damage (Peyman et al., (1995) Adv. Drug. Delivery Rev. 16: 107-123).

Cigarette smoking has been shown to be the most important environmental risk factor for AMD in humans (Tomany et al., Ophthalmology, 2004, 111:1280-1287) and passive smoking has been linked to myopia in children (Stone et al., (2001) Investigative Ophth. Vis. Sci. 42(3):557-565; Stone et al., (2001) Investigative Ophth. Vis. Sci. 47(10):4277-4287; U.S. Pat. App. No. 2003-0096831). In experimental animal models, nicotine has been shown to increase choroidal neovascularization, an effect mediated through activation of nicotinic acetylcholine receptors (nAChR) on endothelial cells (Suñier et al., Invest Ophthalmol Vis Sci., 2004, 45(1): 311-317). This pro-angiogenic effect of nicotine has been shown to be blocked by the nAChR antagonist, hexamethonium. These results indicate that activation of nAChRs plays a significant role in the pathological angiogenesis associated with AMD, and that inhibition of this pathway may inhibit progression of the disease.

Nicotine stimulates endothelial nAChRs to induce endothelial cell proliferation (Villablanca et al., J. Appl. Physiol. 1988, 2089-2098), mobilization and tube formation in vitro (Cooke et al., J. Clin. Invest., 2002, 110:527-536; U.S. Pat. Nos. 6,720,340, 6,417,205) and it has been reported that the maximal effect of nicotine occurs at concentrations similar to those achieved in smokers i.e., 10-100 nM. Nicotine also increases endothelial cell migration, an important event in angiogenesis (U.S. Pat. Nos. 6,720,340, 6,417,205; WO 01/08684; WO 01/08683. This effect of nicotine of increasing endothelial cell migration can be blocked by mecamylamine, a known nAChR antagonist, which has previously been approved by the U.S. Food and Drug Administration for use in the treatment of hypertension. Agents such as mecamylamine that antagonize the endothelial nAChR could represent a novel class of drugs for use in the treatment of diseases characterized by abnormal angiogenesis, such as neovascular or exudative AMD (WO 03/068208; U.S. Pat. App. No. 2003/0216314).

Abnormal angiogenesis and/or neovascularization leading to proliferative retinopathies are believed to mediate other serious conditions affecting the eye and visual acuity. For example, conditions including diabetic retinopathy, retinopathy of prematurity (WO 03/068208; U.S. Pat. App. No. 2003/0216314) and retinopathy associated with sickle cell disease are each believed to be associated with abnormal angiogenesis, neovascularization or combinations thereof.

Mecamylamine has been marketed since the late 1950s for the treatment of hypertension. In normotensive subjects, mecamylamine can cause orthostatic hypotension with a concomitant increase in heart rate. Frequently reported adverse effects associated with systemic mecamylamine administration include constipation, dry mouth, blurred vision from impaired accommodation, weakness, fatigue, cycloplegia, mydriasis (dilated pupil), decreased libido, and urinary retention, as well as CNS disturbance such as tremor, hypersomnia, sedation, convulsion, seizures, choreiform movements, insomnia, mental aberrations, depression, and altered mentation.

In view of these dose-limiting side effects associated with mecamylamine, particularly with systemic delivery of mecamylamine, targeted delivery of the mecamylamine to ocular tissue, for example topical delivery to the surface of the eye, would be highly desirable and could diminish, if not entirely eliminate, the systemic side effects of ganglionic blockade. Additionally, treatment methods and formulations that avoid the use of intra-ocular injection to treat conditions associated with proliferative retinopathies would likely increase patient compliance with treatment regimens as well as reducing the costs associated with administration of injections under local anesthetic, and reducing discomfort to the patient caused by the injection itself and complications associated with intra-ocular injection (Peyman et al., (1995) Adv. Drug. Delivery Rev. 16: 107-123; Tojo et al. (2001) Adv. Drug. Delivery Rev. 52: 17-24).

Thus, methods and formulations utilizing topical ocular administration of therapeutically effective mecamylamine (or pharmaceutically acceptable salts thereof) have many advantages, from standpoint of efficacy, cost, side effects, complications and patient comfort. Such advantages are even more important in the treatment of retinopathy of prematurity in premature infants, as the side effects of drugs, difficulties and complications associated with intra-ocular injection are increased in premature infants due to a number of factors, including the small size of the infant eye, the immaturity of the immune system and the trauma occasioned by such injections.

One of the first successful topical ocular formulations was an in situ gel formulation of timolol, a beta-blocker used to treat glaucoma. The gel formulation has been marketed by Merck & Co. as TIMOPTIC® and is a formulation of timolol and GELRITE® gel, a gellan gum-based gel, which was originally developed as a gelling agent for use in culture media and food products (U.S. Pat. No. 4,861,760). Other gel-based topical ocular formulations include xanthan gum-based gels (U.S. Pat. Nos. 6,174,524 and 6,264,935), which also disclose formulations for the treatment of glaucoma. Other ocular formulations include various components such as polymers or components that complex with the drug active (e.g., U.S. Pat. Nos. 6,159,458, U.S. Pat. App. Pub. Nos. 2005/0084534, 2005/0031697, 2005/0255144). U.S. Pat. No. 6,174,524 also suggests use with timolol, anti-inflammatory agents, growth factors, immunosuppressive agents and other anti-glaucoma agents. In part, it is believed that the success of these formulations is due to the targeted region of treatment, as glaucoma is a condition affecting the anterior region of the eye and thus, to be effective, the drug does not have to reach the posterior region of the eye, thereby traversing additional structures within the eye and being exposed for longer periods to the clearance mechanisms within the eye.

However, despite the success of TIMOPTIC® and intensive research in the field, the development of topical ocular formulations of other drugs has proven difficult and unpredictable, particularly the development of formulations capable of delivering therapeutically effective amounts of drug to the posterior regions of the eye, including the posterior tissues such as the choroid and retina.

For many years researchers have attempted to identify the various mechanisms by which drugs are delivered to the eye in general, and the posterior region of the eye in particular (for example, the retina and/or choroid). The areas of investigation include the barrier functions (e.g., transmembrane flux, etc.) of the various eye tissues and fluids (e.g., retinal pigment epithelium, cornea, retina, choroid, conjunctiva, vitreous body, aqueous humor, etc.), routes of clearance (e.g., clearance from the various eye tissues and/or fluids), clearance due to lacrimal drainage, precorneal tear film, systemic absorption, blood-ocular barrier (from the back of the eye), reflex tearing, etc. (See, for example, Peyman et al., ibid; Lang et al., (1995) Adv. Drug. Delivery Rev. 16: 39-43; Dey et al. (2005) Expert Opin. Drug Deliv. 2(2): 201-204; Järvinen et al. (1995) Adv. Drug. Delivery Rev. 16: 3-19; Pitkanen et al., (2005) Investigative Ophthalmol. Vis. Sci. 46(2): 641-646). Whether or not a particular drug can be absorbed sufficiently through one or more of these barriers and avoid elimination through the processes which clear exogenous materials from the eye in order to deliver an effective amount of drug to the posterior region of the eye depends on a multiplicity of factors, including the physicochemical properties of the drug itself (e.g., size, structure, ionic/charge state, lipophilicity, hydrophilicity, etc.), as well as the interplay between the drug and each of the components in the formulation in which it is administered and the characteristics of the formulation non-drug components (e.g., viscosity, pH, ionicity, etc.). (See, for example, Lang et al., (1995) ibid; Dey et al. (2005) ibid). Unsurprisingly, the multiplicity of factors, which are difficult to model accurately in vitro, has hindered the development of guidelines or the prediction of what drugs, or types of drugs, can be successfully developed for topical administration to the posterior regions of the eye. Even in vivo model studies are difficult to perform accurately and can lead to conflicting results (Maurice (2002) Survey of Ophthalmology 47(Supp. 1): S41-S52). Thus, it is not at all unexpected that a topical method of treatment for conditions associated with proliferative retinopathy, which affects the posterior tissues of the eye, are not, as yet, commercially available.

Quite unexpectedly, in view of the difficulties and unpredictability associated with successful development of topically administered ocular formulations, it has been found and is further described herein, that mecamylamine, when formulated for topical administration, can be delivered to the posterior region of the eye in amounts considered to be therapeutically effective for the treatment or prevention of ocular conditions mediated by angiogenesis and/or abnormal neovascularization in the posterior tissues (e.g., retina, choroid) of the eye, for example, proliferative retinopathies, including diabetic retinopathy, retinopathy of prematurity, retinal neovascularization due to macular degeneration, etc. Even more surprisingly, administration of mecamylamine via topical ocular delivery results in extremely low levels of mecamylamine in plasma (and in red blood cells), while preferentially delivering high levels of mecamylamine to the target tissue in the posterior of the eye (e.g., the retina and choroid).

The formulations and methods described herein may also be used to deliver mecamylamine to other tissues of interest in the eye and the fluids of the eye (which may also be affected by neovascularization, abnormal angiogenesis or combinations thereof). Tissues of interest throughout the eye include the anterior tissues of the eye, when affected by angiogenic disorders such as corneal neovascularization, pterygium, post-corneal transplant neovascularization, rubeosis iridis, neovascular glaucoma, etc.; as well as the posterior tissues of the eye when affected by angiogenic disorders affecting the eye fluids, retinal or choroidal tissues such as age related macular degeneration or diabetic retinopathy.

In view of the numerous well-documented side effects associated with systemic administration (e.g., parenteral (e.g., intravenous, etc.) or oral) of mecamylamine, the low levels of mecamylamine in plasma and high levels of mecamylamine in the posterior tissues and anterior tissues (where the conditions described herein are manifested) observed with topical ocular administration suggests that the side effects associated with systemic administration will be greatly reduced or absent at therapeutic doses of mecamylamine when topically administered, and these conclusions are supported by results from non-human animal models and clinical trials on humans as described in greater detail below and in the examples. When formulated with particular gel-forming polymers, these characteristics of mecamylamine can be further enhanced, preferentially increasing the relative amount of mecamylamine deposited to the tissues of interest while further minimizing the amount of mecamylamine in plasma, although data to date suggests that the solution formulation (free of gel-forming/polymeric components) is well tolerated in animals, including humans, and provides levels of mecamylamine to the tissues of interest that will be efficacious for use in treatment and/or prevention of the conditions described herein.

The advantages of being able to administer mecamylamine, shown to be effective in reducing pathological or unwanted angiogenesis (WO 03/068208; U.S. Pat. App. No. 2003/0216314), in a local (topical ocular), site-specific manner to the eye at therapeutically effective doses are numerous and immediately apparent over both the current standard of care, which usually includes intra-ocular injections, and systemic (e.g., oral, intravenous, etc.) administration of mecamylamine. Not only will patients suffer less discomfort and pain than that associated with the administration of any drug given intra-ocularly, but the complications related to intra-ocular injections will be absent, mecamylamine-specific side effects will be minimized or eliminated, medical costs related to both complications and complex intra-ocular administration will be reduced and, in view of these advantages, patient compliance is also likely to increase. Thus, it is apparent that effective methods and formulations for the delivery of mecamylamine, or pharmaceutically acceptable salts thereof, to the posterior regions of the eye for the treatment and/or prevention of the conditions described herein will be highly beneficial and are currently needed. The formulation will also likely be effective for treating angiogenic disorders of the anterior tissues of the eye, such as corneal neovascularization, pterygium, post-corneal transplant neovascularization, rubeosis iridis, neovascular glaucoma, etc.

### BRIEF SUMMARY OF THE INVENTION

Provided herein are formulations of mecamylamine formulated for topical ocular delivery, including pharmaceutical formulations, kits and methods of making and using the formulations.

In one aspect are provided methods for treating or preventing conditions mediated by neovascularization, abnormal angiogenesis, vascular permeability, or combinations thereof, of posterior tissues, anterior tissues or fluids of the eye comprising topically applying to one or both eyes of an individual in need thereof a formulation comprising mecamylamine, or a pharmaceutically acceptable salt thereof, and a carrier suitable for topical administration to the eye, wherein the mecamylamine or a pharmaceutically acceptable salt thereof is present in the formulation in an amount sufficient to deliver a therapeutically effective amount of mecamylamine to one or more of the posterior or anterior tissues or fluids of the eye for the treatment or prevention of conditions mediated by neovascularization, abnormal angiogenesis, vascular permeability, or combinations thereof, of posterior tissues, anterior tissues or fluids of the eye (step a).

In some embodiments are provided methods for treating or preventing conditions mediated by neovascularization, abnormal angiogenesis, vascular permeability, or combinations thereof, of posterior tissues of the eye comprising topically applying to one or both eyes of an individual in need thereof a formulation comprising mecamylamine, or a pharmaceutically acceptable salt thereof, and a carrier suitable for topical administration to the eye, wherein the mecamylamine or pharmaceutically acceptable salt thereof is present in the formulation in an amount sufficient to deliver a therapeutically effective amount of mecamylamine to one or more of the posterior tissues of the eye for the treatment or prevention of conditions mediated by neovascularization, abnormal angiogenesis, vascular permeability, or combinations thereof, of posterior tissues of the eye (step a). In some embodiments, the condition(s) is mediated by retinal neovascularization. In certain embodiments, the condition(s) is mediated by choroidal neovascularization. In certain embodiments, the condition is a proliferative retinopathy.

In certain embodiments, when the formulation is topically administered to a rabbit eye, the ratio of the concentration of mecamylamine present in choroidal and retinal tissue, measured in units of ng/g, to the concentration of mecamylamine in plasma measured in units of ng/mL ([ng/g mecamylamine choroidal+retinal tissue]: [ng/mL plasma]) is at least about 40:1. In some embodiments, the ratio is at least about 80:1. In others, the ratio is at least about 300:1. In particular embodiments, the ratio is from about 40:1 to about 1000:1. In some embodiments, the ratio is from about 40:1 to about 1500:1. In some embodiments, the ratio is from about 40:1 to about 2000:1.

In certain embodiments, when the formulation is topically administered to a rabbit eye, the ratio of the concentration of mecamylamine present in choroidal and retinal tissue, measured in units of ng/g, to the concentration of mecamylamine in plasma measured in units of ng/mL ([ng/g mecamylamine choroidal+retinal tissue]: [ng/mL plasma]) is at least about 20:1. In some embodiments, the ratio is at least about 25:1, about 30:1 or about 35:1. In particular embodiments, the ratio is from about 20:1 to about 1000:1. In some embodiments, the ratio is from about 20:1 to about 1500:1. In some embodiments, the ratio is from about 20:1 to about 2000:1.

In some embodiments, when the formulation is topically administered to a rabbit eye, the ratio of the concentration of mecamylamine in the choroidal and retinal tissue, measured as the area under the curve (AUC), measured in units of ng/g-hr, versus the concentration of mecamylamine in plasma, measured as the area under the curve (AUC) and measured in units of ng/mL-hr, is at least about 100:1 ([mecamylamine choroida+retinal tissue (ng/g-hr)]: [mecamylamine plasma (ng/mL-hr)]. In some embodiments, the ratio is at least about 50:1. In some embodiments, at least about 80:1, at least about 90:1, or at least about 100:1.

In some embodiments are provided methods for treating or preventing conditions mediated by neovascularization, abnormal angiogenesis, vascular permeability, or combinations thereof, of anterior tissues of the eye comprising topically applying to one or both eyes of an individual in need thereof a formulation comprising mecamylamine, or a pharmaceutically acceptable salt thereof, and a carrier suitable for topical administration to the eye, wherein the mecamylamine or a pharmaceutically acceptable salt thereof is present in the formulation in an amount sufficient to deliver a therapeutically effective amount of mecamylamine to one or more of the anterior tissues or fluids of the eye for the treatment or prevention of conditions mediated by neovascularization, abnormal angiogenesis, vascular permeability, or combinations thereof, of anterior tissues of the eye.

In some embodiments, when the formulation is topically administered to a rabbit eye, the ratio of the concentration of mecamylamine present in corneal tissue, measured in units of ng/g, to the concentration of mecamylamine in plasma measured in units of ng/mL ([ng/g mecamylamine corneal tissue]: [ng/mL plasma]) is at least about 100:1. In some embodiments, the ratio is at least 800:1. In certain embodiments, the ratio is at least about 1000:1. In some embodiments, 1500:1. In some, the ratio is from about 100:1 to about 4000:1. In certain embodiments, the ratio is from about 100:1 to about 3000:1. In some, the ratio is from about 1000:1 to about 4000:1. In certain embodiments, the ratio is from about 1000:1 to about 3000:1.

In some embodiments, when the formulation is topically administered to a rabbit eye, the ratio of the concentration of mecamylamine in the cornea, measured as the area under the curve (AUC), measured in units of ng/g-hr, versus the concentration of mecamylamine in plasma, measured as the area under the curve (AUC) and measured in units of ng/mL-hr, is at least about 100:1 ([mecamylamine cornea (ng/g-hr)]: [mecamylamine plasma (ng/mL-hr)]. In some embodiments, the ratio is at least about 800:1. In some embodiments, at least about 1000:1 or at least about 1500:1.

In some embodiments, when the formulation is topically administered to a rabbit eye, the ratio of the concentration of mecamylamine in the aqueous humor, measured in units of ng/mL, versus the concentration of mecamylamine in plasma, measured in units of ng/mL, is at least about 50:1 ([mecamylamine aqueous humor (ng/mL)]: [mecamylamine plasma (ng/mL)]). In some embodiments, the ratio is at least about 70:1. In some embodiments, at least about 100:1 or at least about 150:1.

In some embodiments, when the formulation is topically administered to a rabbit eye, the ratio of the concentration of mecamylamine in the aqueous humor, measured as the area under the curve (AUC), measured in units of ng/mL-hr, versus the concentration of mecamylamine in plasma, measured as the area under the curve (AUC) and measured in units of ng/mL-hr, is at least about 50:1 ([mecamylamine aqueous humor (ng/mL-hr)]: [mecamylamine plasma (ng/mL-hr) ]. In some embodiments, the ratio is at least about 90:1. In some embodiments, at least about 100:1 or at least about 150:1.

In some embodiments, when the formulation is topically administered to a rabbit eye, the mean maximum concentration of mecamylamine in plasma is less than about 70 ng/mL. In certain embodiments, the mean maximum concentration of mecamylamine in plasma is less than about 50 ng/mL. n certain embodiments, the mean maximum concentration of mecamylamine in plasma is less than about 25 ng/mL. n certain embodiments, the mean maximum concentration of mecamylamine in plasma is less than about 10 ng/mL. n certain embodiments, the mean maximum concentration of mecamylamine in plasma is less than about 5 ng/mL.

In some embodiments, when the formulation is topically administered to a rabbit eye, the total concentration of mecamylamine in plasma measured as the area under the curve is less than about 100 ng/mL-hr. In some embodiments, when the formulation is topically administered to a rabbit eye, the total concentration of mecamylamine in plasma measured as the area under the curve is less than about 85 ng/mL-hr.

In some embodiments, the carrier comprises water. In certain embodiments, the formulation is substantially free of surfactant. In some embodiments, the formulation further includes one or more of a preservative or a surfactant. In certain of these embodiments, the formulation includes a preservative. In some embodiments, the preservative is selected from the group consisting of benzalkonium chloride, benzethonium chloride, chlorhexidine, chlorobutanol, methylparaben, phenylethyl alcohol, propylparaben, thimerosal, phenylmercuric nitrate, phenylmercuric borate, and phenylmercuric acetate. In particular embodiments, the preservative is benzalkonium chloride. In some embodiments, the carrier further comprises one or more tonicity agent(s). In particular embodiments, the one or more tonicity agent(s) is a polyol. In some embodiments, the polyol is a sugar alcohol, trihydroxy alcohol, propylene glycol or polyethylene glycol. In certain embodiments, the one or more tonicity agent(s) is mannitol, glycerin or a combination thereof. In some embodiments, when the formulation is topically administered to a rabbit eye, the mean maximum concentration of mecamylamine in plasma is less than about 70 ng/mL. In particular embodiments, the mean maximum concentration of mecamylamine in plasma is less than about 50 ng/mL. In some embodiments, the mean maximum concentration of mecamylamine in plasma is less than about 25 ng/mL. In particular embodiments, the mean maximum concentration of mecamylamine in plasma is less than about 10 ng/mL. In particular embodiments, the mean maximum concentration of mecamylamine in plasma is less than about 5 ng/mL. In some embodiments, the formulation may also include a chelating agent. In some embodiments, the formulation also includes a chelating agent, one or more preservatives, one or more buffering agents, and one or more tonicity agents. In some embodiments, the formulation also includes a chelating agent, one or more preservatives, one or more tonicity agents, one or more buffering agents and is free of polymer. In some embodiments, the formulation also includes a chelating agent, one or more preservatives, and one or more tonicity agents. In some embodiments, the formulation also includes a chelating agent, one or more preservatives, one or more tonicity agents and is free of polymer. In certain embodiments, the carrier may include a viscosity-increasing agent. In some embodiments, the viscosity-increasing agent is selected from the group consisting of water-soluble cellulose derivatives, polyvinyl alcohol, polyvinyl pyrrolidone, chondroitin sulfate, hyaluronic acid, and soluble starches. In certain embodiments, the viscosity-increasing agent is a water-soluble cellulose derivative. In some embodiments, the viscosity-increasing agent is hypromellose. In some embodiments, the formulation is substantially free of polymer. In some embodiments, the formulation is substantially free of viscosity-increasing agent(s) (e.g., carboxymethylcellulose, polyanionic polymers, etc.). In some embodiments, the viscosity of the formulation is about the same as the viscosity of a saline solution containing the same concentration of mecamylamine (or a pharmaceutically acceptable salt thereof). In certain of these embodiments, the formulation is isotonic. In some embodiments, the formulation is substantially free of surfactant. In some embodiments, the formulation may further include a chelating agent. In certain embodiments, the chelating agent is edetate disodium (dihydrate). In some embodiments, the individual is a human.

In some embodiments, the formulation includes mecamylamine (or a pharmaceutically acceptable salt thereof), a chelating agent, and a preservative, where the carrier is water. In certain embodiments, the preservative is benzalkonium chloride, and the chelating agent is edetate disodium (dihydrate). In some embodiments, the formulation includes mecamylamine (or a pharmaceutically acceptable salt thereof), a chelating agent, one or more buffering agents, and a preservative, where the carrier is water. In some of these embodiments, the preservative is benzalkonium chloride, the buffering agents are sodium phosphate monobasic monohydrate and sodium phosphate dibasic heptahydrate, and the chelating agent is edetate disodium (dihydrate). In some embodiments, the formulations additionally include salt (e.g., NaCl). In some embodiments, the formulation includes from about 0.01% to about 4% mecamylamine (or pharmaceutical acceptable salt thereof) (w/v). In some embodiments, from about 0.01% to about 3% mecamylamine (or pharmaceutical acceptable salt thereof) (w/v). In some embodiments, from about 0.03% to about 3% mecamylamine (or pharmaceutical acceptable salt thereof) (w/v). In some embodiments, about 0.01% mecamylamine (or pharmaceutical acceptable salt thereof) (w/v). In some embodiments, about 0.03% mecamylamine (or pharmaceutical acceptable salt thereof) (w/v). In some embodiments, about 0.3% mecamylamine (or pharmaceutical acceptable salt thereof) (w/v). In some embodiments, about 1% mecamylamine (or pharmaceutical acceptable salt thereof) (w/v). In certain of these embodiments, the formulation is substantially free of polymers (e.g., gel-forming polymers, viscosity-enhancing agents, etc.). In some of these embodiments, the formulation includes a viscosity-increasing agent. In some embodiments, the formulation is substantially free of viscosity-increasing agent(s) (e.g., carboxymethylcellulose, polyanionic polymers, etc.). In some embodiments, the formulation is substantially free of gel-forming polymers. In some embodiments, the viscosity of the formulation is about the same as the viscosity of a saline solution containing the same concentration of mecamylamine (or a pharmaceutically acceptable salt thereof).

In some embodiments, the formulation comprises from about 0.001% to about 6% (w/v) mecamylamine or a pharmaceutically acceptable salt thereof. In some embodiments, the formulation comprises from about 0.001% to about 5% (w/v) mecamylamine or a pharmaceutically acceptable salt thereof. In certain embodiments, from about 0.001% to about 3% (w/v) mecamylamine or a pharmaceutically acceptable salt thereof. In some embodiments, from about 0.03% to about 4% (w/v) mecamylamine or a pharmaceutically acceptable salt thereof. In some embodiments, from about 0.03% to about 3% (w/v) mecamylamine or a pharmaceutically acceptable salt thereof. In some embodiments, from about 0.03% to about 2% (w/v) mecamylamine or a pharmaceutically acceptable salt thereof. In certain embodiments, from about 0.1% to about 1% (w/v) mecamylamine or a pharmaceutically acceptable salt thereof. In certain embodiments, from about 0.05% to about 1% (w/v) mecamylamine or a pharmaceutically acceptable salt thereof.

In some embodiments, the carrier is an aqueous isotonic solution and the formulation may additionally include a chelating agent and a preservative. In certain embodiments, the formulation may also include one or more buffering agents. In some embodiments, the formulation is substantially free of polymers (e.g., gel-forming polymers, viscosity-enhancing agents, etc.). In particular embodiments, the formulation may include a viscosity-increasing agent. In some embodiments, the formulation is substantially free of viscosity-increasing agent(s) (e.g., carboxymethylcellulose, polyanionic polymers, etc.). In some embodiments, the formulation is substantially free of gel-forming polymers. In some embodiments, the viscosity of the formulation is about the same as the viscosity of a saline solution containing the same concentration of mecamylamine (or a pharmaceutically acceptable salt thereof). In some embodiments, the viscosity-increasing agent is selected from the group consisting of water soluble cellulose derivatives, polyvinyl alcohol, polyvinyl pyrrolidone, chondroitin sulfate, hyaluronic acid, and soluble starches. In certain embodiments, the viscosity-increasing agent is a water soluble cellulose derivative. In some embodiments, the viscosity-increasing agent is hypromellose. In some of these embodiments, the formulation comprises from about 0.001% to about 6% (w/v) mecamylamine or a pharmaceutically acceptable salt thereof. In certain embodiments, from about 0.001% to about 3% (w/v) mecamylamine or a pharmaceutically acceptable salt thereof. In some embodiments, from about 0.03% to about 3% (w/v) mecamylamine or a pharmaceutically acceptable salt thereof. In certain embodiments, from about 0.1% to about 1% (w/v) mecamylamine or a pharmaceutically acceptable salt thereof. In some embodiments, the formulation is substantially free of viscosity-increasing agents (e.g., carboxymethylcellulose, polyanionic polymers, etc.). In some embodiments, the viscosity of the formulation is about the same as the viscosity of a saline solution containing the same concentration of mecamylamine (or a pharmaceutically acceptable salt thereof).

In some embodiments, the formulation may include one or more buffering agents. In certain embodiments, the one or more buffering agent(s) is selected from the group consisting of phosphate buffers, citrate buffers, maleate buffers, borate buffers and combinations thereof. In certain embodiments, the buffering agent(s) is a phosphate buffer. In some embodiments, a combination of two phosphate buffers. In particular embodiments, the carrier may additionally include a viscosity-increasing agent. In certain embodiments, the viscosity-increasing agent is hypromellose. In some of these embodiments, the formulation comprises from about 0.001% to about 6% (w/v) mecamylamine or a pharmaceutically acceptable salt thereof. In certain embodiments, from about 0.001% to about 3% (w/v) mecamylamine or a pharmaceutically acceptable salt thereof. In some embodiments, from about 0.03% to about 3% (w/v) mecamylamine or a pharmaceutically acceptable salt thereof. In certain embodiments, from about 0.1% to about 1% (w/v) mecamylamine or a pharmaceutically acceptable salt thereof.

In some embodiments, the carrier comprises an aqueous saline solution. In particular embodiments, the aqueous saline solution is isotonic.

In certain embodiments, the carrier comprises from about 0.03% to about 2% (w/v) of a gel-forming polymer and water, wherein the gel-forming polymer is selected such that when the formulation is topically administered to a rabbit eye the ratio of the concentration of mecamylamine present in choroidal and retinal tissue, measured in units of ng/g, to the concentration of mecamylamine in plasma, measured in units of ng/mL, ([ng/g mecamylamine choroidal+retinal tissue]: [ng/mL plasma]) is at least about 300:1. In some embodiments, the formulation is a gel prior to topical ocular administration. In other embodiments, the formulation forms a gel in situ upon topical ocular administration. In particular embodiments, the gel-forming polymer is a polysaccharide. In certain embodiments, the polysaccharide is gellan gum. In some embodiments, when the formulation is topically administered to a rabbit eye, the mean maximum concentration of mecamylamine in plasma is less than about 70 ng/mL. In some embodiments, less than 50 ng/mL. In some embodiments, the mean maximum concentration of mecamylamine in plasma is less than about 25 ng/mL. In particular embodiments, the mean maximum concentration of mecamylamine in plasma is less than about 10 ng/mL. In particular embodiments, the mean maximum concentration of mecamylamine in plasma is less than about 5 ng/mL. In some embodiments, the carrier further comprises one or more tonicity agent(s). In particular embodiments, the one or more tonicity agent(s) is a polyol. In some embodiments, the polyol is a sugar alcohol, trihydroxy alcohol, propylene glycol or polyethylene glycol. In certain embodiments, the one or more tonicity agent(s) is mannitol, glycerin or a combination thereof. In some of these embodiments, the formulation comprises from about 0.001% to about 6% (w/v) mecamylamine or a pharmaceutically acceptable salt thereof. In certain embodiments, from about 0.001% to about 3% (w/v) mecamylamine or a pharmaceutically acceptable salt thereof. In some embodiments, from about 0.03% to about 3% (w/v) mecamylamine or a pharmaceutically acceptable salt thereof. In certain embodiments, from about 0.1% to about 1% (w/v) mecamylamine or a pharmaceutically acceptable salt thereof.

In certain embodiments, the carrier comprises from about 0.05% to about 2% (w/v) gellan gum. In particular embodiments, the carrier comprises from about 0.1% to about 1% (w/v) gellan gum. In some embodiments, the carrier comprises from about 0.1% to about 0.6% (w/v) gellan gum.

In some embodiments, the formulation is substantially free of surfactant.

In particular embodiments, the formulation further comprises one or more of a preservative or a surfactant.

In certain embodiments, the formulation further comprises a preservative. In certain embodiments the preservative is benzalkonium chloride, benzethonium chloride, chlorhexidine, chlorobutanol, methylparaben, phenylethyl alcohol, propylparaben, thimerosal, phenylmercuric nitrate, phenylmercuric borate, or phenylmercuric acetate. In some embodiments, the preservative is benzalkonium chloride.

In certain embodiments, the carrier may include a viscosity-increasing agent. In some embodiments, the viscosity-increasing agent is selected from the group consisting of water soluble cellulose derivatives, polyvinyl alcohol, polyvinyl pyrrolidone, chondroitin sulfate, hyaluronic acid, and soluble starches. In certain embodiments, the viscosity-increasing agent is a water soluble cellulose derivative. In some embodiments, the viscosity-increasing agent is hypromellose.

In some embodiments, the formulation may further include a chelating agent. In certain embodiments, the chelating agent is edetate disodium (dihydrate).

In some embodiments, the carrier further comprises one or more tonicity agent(s). In particular embodiments, the one or more tonicity agent(s) is a polyol. In some embodiments, the polyol is a sugar alcohol, trihydroxy alcohol, propylene glycol or polyethylene glycol. In certain embodiments, the one or more tonicity agent(s) is mannitol, glycerin or a combination thereof.

In particular embodiments, the formulation comprises from about 0.001% to about 6% (w/v) mecamylamine or pharmaceutically acceptable salt thereof. In some embodiments, the formulation comprises from about 0.001% to about 5% (w/v) mecamylamine or pharmaceutically acceptable salt thereof. In certain of these embodiments, the carrier comprises from about 0.05% to about 1% (w/v) gellan gum and water.

In some embodiments, the formulation includes a pharmaceutically acceptable salt of mecamylamine. In particular embodiments, the salt of mecamylamine is mecamylamine hydrochloride.

In some embodiments, the individual has been identified as having one or more conditions mediated by retinal neovascularization, choroidal neovascularization, abnormal angiogenesis, vascular permeability, or combinations thereof, of posterior tissues of the eye (including conditions associated with proliferative retinopathy of the eye). In some embodiments, the individual has been identified as having a non-neovascular form of macular degeneration. In particular embodiments, the individual has been identified as susceptible to one or more conditions mediated by retinal neovascularization, choroidal neovascularization, neovascularization, abnormal angiogenesis, vascular permeability, or combinations thereof, of posterior tissues of the eye (including conditions associated with proliferative retinopathy of the eye). In some embodiments, the individual has as been identified as being susceptible to a non-neovascular form of macular degeneration.

In certain embodiments, the condition is diabetic retinopathy, retinopathy of prematurity, retinal neovascularization due to macular degeneration, retinopathy associated with mascular edema, or retinopathy associated with sickle cell disease. In particular embodiments, the condition is diabetic retinopathy. In other embodiments, the condition is retinopathy of prematurity. In still other embodiments, the condition is retinal neovascularization due to macular degeneration. In another embodiment, the condition is an age-related maculopathy. In still another embodiment, the condition is age-related macular degeneration. In particular embodiments, the age-related macular degeneration is a neovascular form (e.g. wet form) of age-related macular degeneration.

In some embodiments, the condition is associated with abnormal angiogenesis affecting the anterior tissues of the eye or is a condition involving abnormal angiogenesis affecting both anterior and posterior tissues of the eye. In some embodiments, the condition is associated with abnormal angiogenesis affecting the anterior tissues of the eye. In certain embodiments, the condition is corneal neovascularization, pterygium, post-corneal transplant neovascularization, rubeosis iridis, or neovascular glaucoma. In some embodiments, the condition is an ocular tumor.

In certain embodiments, the condition involves vitreal, retinal or choroidal neovascularization. In some embodiments, the condition is an ocular tumor.

In some embodiments, the condition is associated with abnormal angiogenesis affecting both anterior and posterior tissues of the eye. In some embodiments, the condition is an ocular tumor.

In some embodiments, the individual is a mammal. In certain embodiments the mammal is a primate, rabbit, canine, feline, or rodent. In particular embodiments, the mammal is a primate. In certain embodiments, the primate is a human. In some embodiments, the individual is not experiencing ocular growth. In some embodiments, the individual is an adult.

In certain embodiments, the condition is retinopathy of prematurity and the individual is a human.

In some embodiments, the therapeutically effective amount of mecamylamine is delivered to the retina.

In particular embodiments, the therapeutically effective amount of mecamylamine is delivered to the choroid.

In certain embodiments, the therapeutically effective amount of mecamylamine is delivered to the retina and the choroid.

In some embodiments, the therapeutically effective amount of mecamylamine is delivered to the cornea, iris, trabecular meshwork, sclera or lens.

In certain embodiments, the therapeutically effective amount of mecamylamine is delivered to the cornea.

In certain embodiments, the therapeutically effective amount of mecamylamine is delivered to the sclera.

In certain embodiments, the therapeutically effective amount of mecamylamine is delivered to the lens.

In some embodiments, the therapeutically effective amount of mecamylamine is delivered to the iris.

In certain embodiments, the therapeutically effective amount of mecamylamine is delivered to the trabecular meshwork.

In certain embodiments, the condition is associated with abnormal angiogenesis affecting the anterior tissues of the eye or is a condition involving abnormal angiogenesis affecting both the anterior and posterior tissues of the eye. In some embodiments, the condition is an ocular tumor.

In some embodiments, the condition is corneal neovascularization, pterygium, post-corneal transplant neovascularization, rubeosis iridis, or neovascular glaucoma.

In some embodiments, the application is performed once per day, twice per day, three times per day, four times per day, once every other day, once per week, or twice per week. In particular embodiments, the application is performed once per day or twice per day.

In some embodiments the methods further include a step (b), where step (b) includes administering to the individual an effective amount of a pharmaceutical agent (other than mecamylamine), additional treatment modality, or combinations of the foregoing. Step (b) may be performed prior to, concomitantly with or after step (a). And, in some variations, step (b) may be performed more than once (e.g., twice, three times, etc.) (e.g., both prior to and after step (a), both concomitantly with and after step (a), both prior to and concomitantly with step (a), etc.) For example, in certain variations step (b) may be performed prior to or concomitantly with step (a). In other variations, step (b) may be performed concomitantly with or after step (a). In still other variations, step (b) may be performed prior to or after step (a). In particular variations, step (b) may be performed prior to step (a). In some variations, step (b) may be performed concomitantly with step (a). In certain variations, step (b) may be performed after step (a). Where step (b) includes administration of a combination of a pharmaceutical agent and an additional treatment modality(ies), each may be independently administered prior to, concomitantly with or after step (a). In particular embodiments, step (b) includes a pharmaceutical agent. In certain embodiments, the pharmaceutical agent is an anti-VEGF antibody or fragment thereof. In some embodiments the anti-VEGF antibody is bevacizumab, ranibizumab, or a combination thereof. In some embodiments, the pharmaceutical agent is VEGF antagonist. In particular embodiments, the VEGF antagonist is a VEGF aptamer. In certain of these embodiments, the VEGF aptamer is pegaptanib. In some embodiments, the pharmaceutical agent is a tyrosine kinase inhibitor. In some embodiments, the pharmaceutical agent is a VEGF scavenger (e.g., VEGF TRAP, etc.). In some embodiments, the pharmaceutical agent is a non-steroidal anti-inflammatory drug. In some embodiments, the pharmaceutical agent is a prostaglandin receptor antagonist. In some variations, the pharmaceutical agent is a VEGF scavenger, VEGF antagonist, or tyrosine kinase inhibitor.

In particular embodiments, step (b) includes thermal laser photocoagulation or photodynamic therapy. In certain embodiments, step (b) includes photodynamic therapy. In some embodiments, step (b) includes thermal laser photocoagulation.

In another aspect are provided pharmaceutical formulations for ocular topical delivery of mecamylamine. Thus, in some embodiments are provided pharmaceutical formulations comprising mecamylamine, or a pharmaceutically acceptable salt thereof, water and a gel-forming polymer for ocular topical administration, wherein the gel-forming polymer is selected such that when the formulation is topically administered to a rabbit eye, the ratio of the concentration of mecamylamine present in the choroidal and retinal tissue, measured in units of ng/g, to the concentration of mecamylamine in plasma measured in units of ng/mL ([ng/g mecamylamine choroidal+retinal tissue]: [ng/mL plasma]) is at least about 300:1.

In certain embodiments, the ratio is from about 300:1 to about 1000:1. In particular embodiments, the ratio is at least about 350:1.

In some embodiments, when the formulation is topically administered to a rabbit eye, the mean maximum concentration of mecamylamine in plasma is less than about 70 ng/mL. In certain embodiments, the mean maximum concentration of mecamylamine in plasma is less than about 50 ng/mL. In some embodiments, the mean maximum concentration of mecamylamine in plasma is less than about 25 ng/mL. In particular embodiments, the mean maximum concentration of mecamylamine in plasma is less than about 10 ng/mL. In particular embodiments, the mean maximum concentration of mecamylamine in plasma is less than about 5 ng/mL.

In some embodiments, the formulation comprises from about 0.001% to about 6% (w/v) mecamylamine or a pharmaceutically acceptable salt thereof. In some embodiments, the formulation comprises from about 0.001% to about 5% (w/v) mecamylamine or a pharmaceutically acceptable salt thereof. In certain embodiments, from about 0.001% to about 3% (w/v) mecamylamine or a pharmaceutically acceptable salt thereof. In some embodiments, from about 0.03% to about 4% (w/v) mecamylamine or a pharmaceutically acceptable salt thereof. In some embodiments, from about 0.03% to about 3% (w/v) mecamylamine or a pharmaceutically acceptable salt thereof. In some embodiments, from about 0.03% to about 2% (w/v) mecamylamine or a pharmaceutically acceptable salt thereof. In certain embodiments, from about 0.1% to about 1% (w/v) mecamylamine or a pharmaceutically acceptable salt thereof. In certain embodiments, from about 0.05% to about 1% (w/v) mecamylamine or a pharmaceutically acceptable salt thereof.

In some embodiments, the gel-forming polymer is present at a concentration of from about 0.03% to about 2% (w/v).

In particular embodiments, the formulation is a gel prior to topical ocular administration. In some of these embodiments, the gel-forming polymer is a polysaccharide.

In certain embodiments, the formulation forms a gel in situ upon topical ocular administration. In particular of these embodiments, the gel-forming polymer is a polysaccharide. In some embodiments, the polysaccharide is gellan gum. In certain embodiments, the gel-forming polymer is gellan gum present at a concentration of from about 0.05% to about 2% (w/v). In others the gellan gum is present at a concentration of about 0.1% to about 1% (w/v). In still others, the gellan gum is present at a concentration of about 0.1% to about 0.6% (w/v).

In certain embodiments the formulation is substantially free of surfactant. In some of these embodiments, the formulation includes a preservative.

In certain embodiments the formulations include one or more of a preservative or a surfactant. In particular embodiments the formulations include a preservative.

In some embodiments, the carrier further comprises one or more tonicity agent(s). In particular embodiments, the one or more tonicity agent(s) is a polyol. In some embodiments, the polyol is a sugar alcohol, trihydroxy alcohol, propylene glycol or polyethylene glycol. In certain embodiments, the one or more tonicity agent(s) is mannitol, glycerin or a combination thereof.

In certain embodiments, where a preservative is present, the preservative may be one or more of benzalkonium chloride, benzethonium chloride, chlorhexidine, chlorobutanol, methylparaben, phenylethyl alcohol, propylparaben, thimerosal, phenylmercuric nitrate, phenylmercuric borate, or phenylmercuric acetate. In certain embodiments the preservative is benzalkonium chloride.

In some embodiments, the mecamylamine or pharmaceutically acceptable salt thereof is present at concentration of from about 0.001% to about 6% (w/vol.). In particular embodiments, the mecamylamine or pharmaceutically acceptable salt thereof is present at concentration of from about 0.001% to about 5% (w/vol.). In certain of these embodiments, the gel-forming polymer is gellan gum present at a concentration of from about 0.05% to about 1% (w/v).

In certain embodiments, the formulations contain a pharmaceutically acceptable salt of mecamylamine. In particular embodiments, the salt of mecamylamine is mecamylamine hydrochloride.

In some variations, the mecamylamine, or pharmaceutical acceptable salt thereof, is incorporated in the formulation as substantially pure S-mecamylamine. In some variations the mecamylamine, or pharmaceutical acceptable salt thereof, is incorporated in the formulation as substantially pure R-mecamylamine.

In some embodiments, the formulations also include a pharmaceutical agent, as described herein.

In yet another aspect are provided kits including the topical ocular mecamylamine formulations as described herein. It is intended that the any of the formulations described herein may be included in the kits of the present invention.

In certain embodiments are provided kits including any of the topical ocular mecamylamine formulations described herein, packaging and instructions for use.

In certain embodiments, the formulation is provided in a multi-dose form.

In particular embodiments, the formulation is provided in one or more single unit dose forms.

In some embodiments, sufficient formulation (in either unit dose or multi dose form) is provided for treatment over a period of about 1 day, about 1 week, about 2 weeks, about 3 weeks, about 4 weeks, about 1 month, about 2 months, about 3 months, about 4 months, about 6 months, about 9 months or about 1 year. In particular embodiments, sufficient formulation is provided for about 3 months. In other embodiments, sufficient formulation is provided for about 1 or 2 months.

In some embodiments, the kits include one or more pharmaceutical agents (non-mecamylamine pharmaceutical agents). In certain embodiments, the kits may include one or more non-mecamylamine nicotinic acetylcholine receptor antagonists. In particular embodiments, the pharmaceutical agent is provided in a separate container from the pharmaceutical formulation of mecamylamine, or a pharmaceutically acceptable salt thereof.

In another aspect of the present invention are provided methods of preparing the topical ocular formulations of mecamylamine. These methods generally include mixing the mecamylamine and carrier components (including the gel-forming polymer) in sufficient amounts to prepare a formulation with the desired concentrations of each component.

In particular embodiments are provided methods for the preparation of the topical ocular formulations which include a gel-forming polymer, including the steps of (a) dispersing a gel-forming polymer in an aqueous solution of mecamylamine, or a pharmaceutically acceptable salt thereof; (b) mixing the mixture formed in step (a) to form a solution or gel; and, (c) equilibrating the solution or gel formed in step (b). In some embodiments are provided methods for the preparation of the topical ocular formulations which include a gel-forming polymer, including the steps of (a) dispersing a gel-forming polymer in an aqueous solution of mecamylamine, or a pharmaceutically acceptable salt thereof and (b) mixing the mixture formed in step (a) to form a solution or gel. In some embodiments, the method of preparation further comprises a step, (c) equilibrating the solution or gel formed in step (b).

In some embodiments, the aqueous solution of mecamylamine, or a pharmaceutically acceptable salt thereof, further comprises a pharmaceutical agent, a preservative or a surfactant. In some embodiments, the aqueous solution also includes a preservative. In others, the aqueous solution also includes a surfactant.

In certain embodiments, the solution formed in step (c) forms a gel in situ upon topical ocular administration.

In other embodiments, the solution formed in step (b) or step (c) is a gel prior to topical ocular administration.

In some embodiments, the mixing in step (b) includes stirring.

In particular embodiments, the mixing in step (b) includes heating.

Unless otherwise noted, the formulations of mecamylamine as described herein are intended for use in the methods of treatment and/or prevention as described herein and may be incorporated in the kits described herein. The pharmaceutical formulations described herein may, unless otherwise noted, be made by the methods of preparation as described herein.

In a further aspect of the invention is provided use of the formulations of mecamylamine (e.g., including formulations free of polymers, formulations incorporating viscosity-enhancing agent, formulations incorporating gel-forming polymer, etc.) as described herein in the manufacture of a medicament. Particularly, the manufacture of a medicament for use in the treatment and/or prevention of conditions as described herein. Further, the formulations thereof, variously described herein (e.g., including formulations free of polymers, formulations incorporating viscosity-enhancing agent, formulations incorporating gel-forming polymer, etc.), are also intended for use in the manufacture of a medicament for use in treatment and/or prevention of the conditions and, in accordance with the methods, described herein, unless clearly dictated otherwise by context or specifically noted.

In yet another aspect of the invention are provided the formulations as described herein for use in the treatment and/or prevention of the conditions described herein (e.g., including formulations free of polymers, formulations incorporating viscosity-increasing agent(s), formulations incorporating gel-forming polymer, etc.). For example, formulations for the treatment and/or prevention of the conditions described herein where the carrier includes water or saline. In some embodiments, the carrier may be water and the formulation may further include NaCl. In some embodiments, the formulation may be isotonic. In some embodiments, the formulation may be hypotonic. In others, hypertonic. In some variations, the carrier includes a gel-forming polymer. In some embodiments, the carrier may be an in situ gel-forming polymer. In some embodiments, the formulation is substantially free of gel-forming polymer. In some embodiments, the formulation is substantially free of polymers (e.g., including gel-forming polymers, viscosity-enhancing agents, etc.). In some embodiments, the formulation is substantially free of viscosity-increasing agents (e.g., carboxymethylcellulose, polyanionic polymers, etc.). In some embodiments, the viscosity of the formulation is about the same as the viscosity of a saline solution containing the same concentration of mecamylamine (or a pharmaceutically acceptable salt thereof). In some variations, the carrier includes a viscosity-enhancing agent.

### BRIEF DESCRIPTION OF THE DRAWINGS

FIG. 1 shows the concentration (ng/mL) of mecamylamine in plasma (▪) and vitreous (▴) after intravenous administration of an aqueous parenteral solution of mecamylamine hydrochloride over a 1 hour infusion period to rabbits at a total dose/rabbit of 15 mg/kg.

FIG. 2 shows the concentration (ng/mL) of mecamylamine in plasma (▪) and vitreous (▴) after intravenous administration of an aqueous parenteral solution of mecamylamine hydrochloride over a 6 hour infusion period to rabbits at a total dose/rabbit of 15 mg/kg.

FIG. 3 shows the concentration (ng/g) of mecamylamine in retinal/choroidal tissue from rabbit eyes after a 1 hour (S) or 6 hour (L) intravenous infusion of an aqueous parenteral solution of mecamylamine hydrochloride at a total dose/rabbit of 15 mg/kg.

FIG. 4 shows the concentrations of mecamylamine in plasma (ng/mL), vitreous (ng/mL) and retinal/choroidal tissue (ng/g) for rabbits administered (A) as an intravenous infusion over a period of 6 hours of an aqueous parenteral solution of mecamylamine hydrochloride at a total dose/rabbit of 15 mg/kg and (B) via topical administration to the rabbit eye of an in situ gel-forming solution of 2% mecamylamine hydrochloride at a dose of ˜1 mg/kg/eye.

FIG. 5 shows the concentrations of mecamylamine in plasma (ng/mL), vitreous (ng/mL) and retinal/choroidal tissue (ng/g) for rabbits topically administered (A) an aqueous isotonic ophthalmic solution of 2% mecamylamine hydrochloride at a dose of ˜1 mg/kg/eye and (B) an in situ gel-forming solution of 2% mecamylamine at a dose of ˜1 mg/kg/eye.

FIG. 6 shows the concentration (ng/mL) of mecamylamine over time (t=0 to t=24 hours) after topical administration to rabbits of (A) an aqueous isotonic ophthalmic solution of 2% mecamylamine hydrochloride at a dose of ˜1 mg/kg/eye in the vitreous (▴), plasma (▪) and red blood cells (▴), and (B) an in situ gel-forming solution of 2% mecamylamine at a dose of ˜1 mg/kg/eye in the vitreous (▴), plasma (♦) and red blood cells (▪).

### DETAILED DESCRIPTION OF THE INVENTION

Provided herein are formulations of mecamylamine, or pharmaceutically acceptable salts thereof, formulated for topical delivery to the eye, as well as methods for the treatment and/or prevention of conditions mediated by neovascularization, abnormal angiogenesis, vascular permeability, or combinations thereof, of anterior tissues, posterior tissues and fluids of the eye (including proliferative retinopathies, corneal neovascularization, pterygium, rubeosis, post-corneal transplant neovascularization, vitreal neovascularization, neovascular glaucoma, etc.) using such formulations, kits comprising these formulations and methods for preparing the formulations.

Similarly, also provided herein are formulations of mecamylamine, or pharmaceutically acceptable salts thereof, formulated for topical delivery to the eye, as well as methods for the treatment and/or prevention of conditions mediated by retinal or choroidal neovascularization, abnormal angiogenesis, vascular permeability, or combinations thereof, of posterior tissues of the eye (including proliferative retinopathies) using such formulations, kits comprising these formulations and methods for preparing the formulations. In addition, also provided are methods for the treatment and/or prevention of conditions mediated by neovascularization, abnormal angiogenesis, vascular permeability, or combinations thereof, of anterior tissues of the eye using such formulations, kits comprising these formulations and methods for preparing the formulations

Ocular conditions mediated by neovascularization (retinal and/or choroidal), abnormal angiogenesis, vascular permeability (or combinations of two or more of the foregoing) of the posterior tissues of the eye, for example, proliferative retinopathies, including diabetic retinopathy, retinopathy of prematurity, retinopathy due to sickle cell disease, retinopathy associated with macular edema, and retinal neovascularization due to macular degeneration, as well as the additional conditions described herein, are serious conditions affecting millions of people in the U.S. and worldwide and which usually lead to significant vision loss and even blindness. Indeed, age-related macular degeneration is one of the leading causes of blindness in the elderly and retinopathy of prematurity can lead to varying degrees of life-long vision impairment for infants born prematurely. Vision impairment, either partial or total loss of vision or visual acuity, adversely affects the quality of life of the individual, often restricting the mobility, productivity and independence of affected individuals.

Retinopathy of prematurity (ROP) is a pathological neovascularization of the retina that occurs in premature infants. The significance of this disease is that it leads to poor visual acuity or blindness in 45-65% of these children (“Cryotherapy for Retinopathy of Prematurity Cooperative Group: 15-year outcomes following threshold retinopathy of prematurity: final results from the multicenter trial of cryotherapy for retinopathy of prematurity” (2005) Arch Ophthalmol. 123: 311-318). For those neonates weighing≦1250 grams, the prevalence of ROP is common. In the Early Treatment for Retinopathy of Prematurity (ETROP) trial, 68% of premature infants≦1250 grams developed ROP (Early Treatment for Retinopathy of Prematurity Cooperative Group: The incidence and course of retinopathy of prematurity: findings from the early treatment for retinopathy of prematurity study. Pediatrics (2005) 116: 15-23). Unfortunately, since an earlier natural history study conducted almost 20 years ago (Palmer et al. “Incidence and early course of retinopathy of prematurity” (1991) Ophthalmology 98:1628-1640), the incidence of ROP, time of onset, rate of progression, and time of onset of pre-threshold disease has changed little. Accordingly, there is a compelling need to gain new insights and develop new therapeutic approaches for this disease.

Angiogenic disorders that affect the anterior tissues of the eye include abnormal angiogenesis involving the cornea due to a pathologic ingrowth of vessels from the limbal vascular plexus into the cornea. This abnormal vessel growth may be due to infection, contact lens wear, trauma, chemical burns, immunologic diseases, degeneration or intraocular events such as uveitis, glaucoma and pthisis bulbi. Typical treatment is with topical corticosteroids which may be combined with corneal laser photocoagulation. Pterygium is a raised, wedge-shaped growth of the conjunctiva. It is most common among those who live in tropical climates or spend a lot of time in the sun. Symptoms may include irritation, redness, and tearing. Pterygiums are nourished by tiny capillaries that supply blood to the tissue. In some cases, this abnormal fibrovascular tissue grows over the central cornea and affects the vision. Typical treatment includes artificial tears, corticosteroids or surgery. Rubeosis iridis is usually a complication of diabetes, and involves abnormal vessel growth in the iris and ciliary body, which can lead to glaucoma. Treatment includes photocoagulation and cryocoagulation.

As noted previously, the development of topical ocular formulations is highly unpredictable (Lang et al., (1995) Adv. Drug. Delivery Rev. 16: 39-43; Dey et al. (2005) Expert. Opin. Drug Deliv. 2(2): 201-204; Järvinen et al. (1995) Adv. Drug. Delivery Rev. 16: 3-19; Maurice (2002) Survey of Ophthamology 47(Supp. 1): S41-S52; Paulsson, M., (2001) “Controlled Release Gel Formulations for Mucosal Drug Delivery” Acta Universitatis Upsaliensis. Comprehensive Summaries of Uppsala Dissertations from the Faculty of Pharmacy 259. 52 pp. Uppsala. ISBN 91-554-5173-X.) and dependent on a multiplicity of factors. Thus, unsurprisingly, to date, effective, non-invasive drug therapies capable of arresting or ameliorating these conditions without causing significant side effects and/or complications have not been successfully developed. Current drug therapies for most proliferative retinopathies commonly involve the use of intra-ocular injection (Peyman et al., (1995) Adv. Drug. Delivery Rev. 16: 107-123; Tojo et al. (2001) Adv. Drug. Delivery Rev. 52: 17-24), for example sub-conjuntival or intra-vitreal injections, which aim to increase the amount of drug delivered to the posterior region of the eye. These procedures, however, have numerous disadvantages, including the requirement for repeated injections, which each carry the risk of complications, such as, retinal detachment, vitreous hemorrhage, endophthalmitis, and lens damage. Additionally, intra-vitreal injections also generally require specialized training to administer and require the use of local anesthesia (Peyman et al., (1995) Adv. Drug. Delivery Rev. 16: 107-123), thus substantially increasing the cost of treatment.

The development of less invasive, for example topical, delivery routes has been hampered by the complexity of the eye and its numerous mechanisms for excluding exogenous substances, such as the mechanisms described above. Indeed, it has been estimated that in the application of eye drops less than 5% of the drug applied penetrates the cornea and reaches intra-ocular tissues (Jäirvinen et al., (1995) ibid). Reported data for the delivery of drugs and their formulation for delivery to the posterior region of the eye is extremely limited, and, it appears, often flawed (Maurice (2002) ibid) and, thus, generalization regarding the likelihood of success and the suitability of a particular drug for topical ocular delivery is not scientifically reasonable, while guidelines for the development of particular formulations to enable or enhance such delivery are not currently available.

However, surprisingly and in spite of these difficulties, the present invention relates to pharmaceutical formulations and methods of using the formulations of mecamylamine (or pharmaceutically acceptable salts thereof) for topical administration to the eye, with tolerable, indeed, minimal, side effects for the treatment of conditions mediated by retinal and/or choroidal neovascularization, abnormal angiogenesis, vascular permeability, or combinations thereof (e.g., combinations of two or more of the foregoing), of posterior tissues of the eye (including proliferative retinopathies), as described in greater detail herein. These formulations and methods should vastly enhance the quality of life and the outlook of millions of individuals, with the additional advantages of providing treatment with lower risks of complications and side effects, as well as reduced costs for administration, while likely also increasing patient compliance and successful completion of the course of treatment due to the decreased costs and discomforts compared to currently available drug therapies.

As described in more detail in the examples, a given dose of mecamylamine, or a pharmaceutically acceptable salt thereof, when administered topically in a formulation suitable for use in the eye preferentially delivers a greater concentration of mecamylamine to the posterior tissues in the eye (e.g., the retina and choroid) relative to the concentration of mecamylamine appearing in plasma, compared to a given amount of mecamylamine administered systemically (e.g., intravenously, etc.). It has also been observed that high concentrations of mecamylamine are also present in the cornea and other anterior tissues (and fluid (e.g., aqueous humor)) after topical ocular administration of mecamylamine.

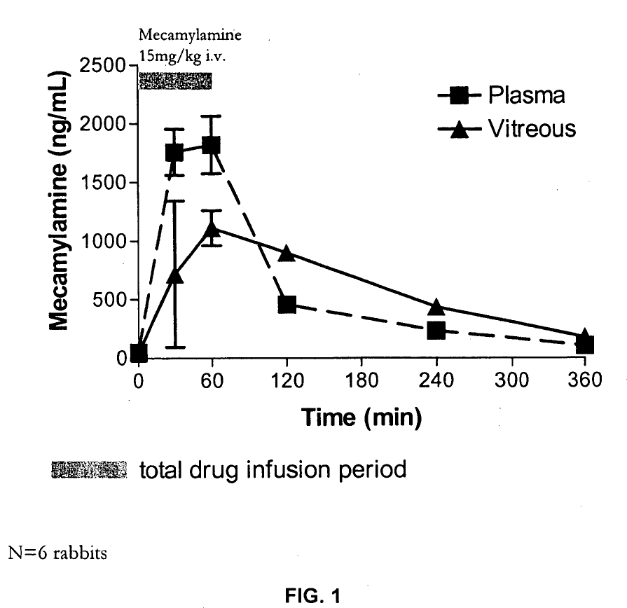

This surprising result can also be conveyed by comparison of the ratios for the concentration of mecamylamine in retinal/choroidal tissue (ng/g):concentration of mecamylamine in plasma (ng/mL) for both systemically administered mecamylamine and mecamylamine administered topically through the eye. A comparison of these ratios is set forth in Example 6, Table 6. The underlying data in support of Table 6 are described in Examples 2, 5 and 6, with the results presented graphically in FIGS. 1-6.

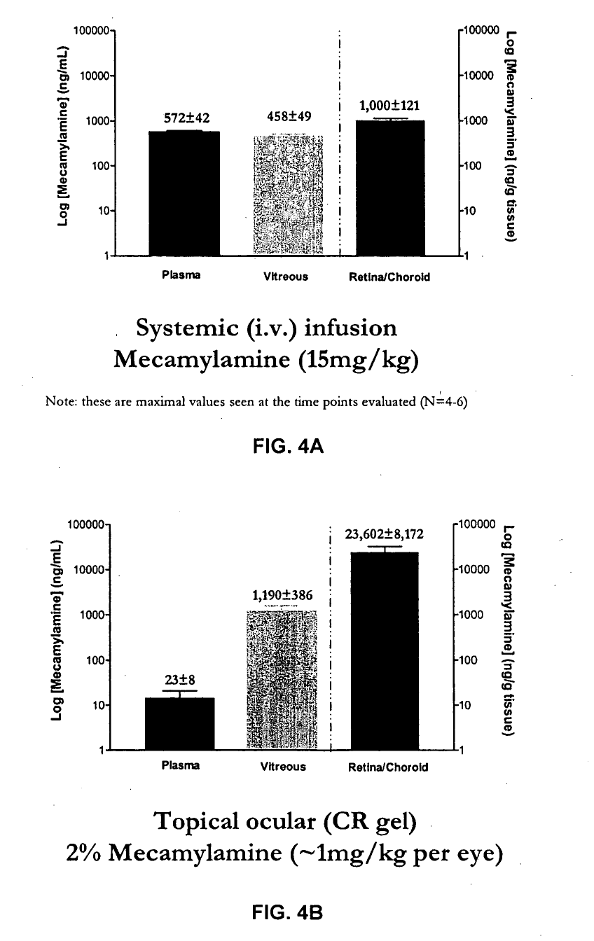

In particular, comparison of FIGS. 4A and 4B/5A and review of Table 6 clearly show that mecamylamine (or a pharmaceutically acceptable salt thereof), when administered topically, is preferentially targeted to the retinal/choroidal tissues (a ratio of at least about 80:1 (isotonic solution) versus when mecamylamine is administered systemically (a ratio of about 1.4:1 in short infusion and about 2.1:1 in long infusion, respectively). Such partitioning of the mecamylamine in the retinal/choroidal tissues is unexpected in view of the work in the field and overcomes the difficulties known in the field with regard to successful development of topical ocular formulations. Namely, mecamylamine, when applied topically in formulations suitable for the eye, is able to be delivered in high enough concentrations to the posterior tissues of the eye to be therapeutically effective, while not being cleared from the eye or absorbed systemically in large amounts (e.g., being detectable at high concentrations in plasma).

Additionally, when mecamylamine is formulated with particular gel-forming polymers (e.g., GELRITE® (gellan gel)), the ratio of the mecamylamine concentration in retina/choroid:plasma can be increased further, thus additionally enhancing the preferential delivery of mecamylamine to the region of interest (i.e., the posterior region of the eye (e.g., the retina and/or choroid)), where the conditions described here are manifested, while maintaining very low levels of mecamylamine in the plasma (e.g., maintaining the minimization and/or elimination of side effects). This increase in the ratio is even more unexpectedly large for the gel-forming polymer carrier when compared to the isotonic solution for topical administration, as shown in Table 6. While the topically applied isotonic solution of mecamylamine hydrochloride showed a surprising large ratio of mecamylamine tissue:plasma concentration of at least about 80:1 to about 204:1, the in situ gel-forming agent GELRITE® (gellan gel) solution of mecamylamine hydrochloride at the same dose exhibited a ratio of at least about 497:1.

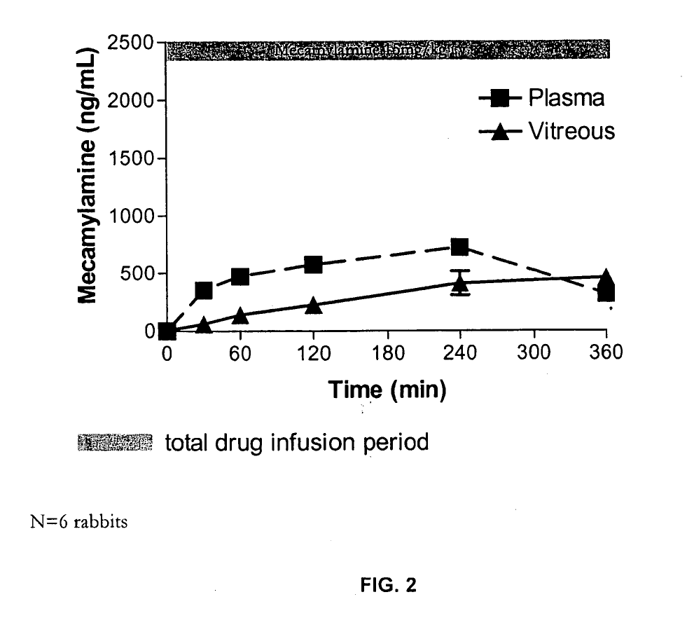

For reference, when Tan et al. (J. Glaucoma (2002) 11: 134-142) investigated the disposition of timolol in the retina and plasma of rats that were topically administered a single dose of timolol to the eye, a ratio of the concentration of timolol in the retina:plasma of approximately 3:1 (see FIG. 2, time point approximately 1 hour) was observed. This ratio is at least approximately 25-fold less than that presently observed for topically administered mecamylamine isotonic solution and at least about 165-fold less than the ratio observed for the topically administered mecamylamine in situ gel-forming solution. Thus, particularly in view of the previously available data for other active agents, the data obtained for mecamylamine are quite surprising and unexpected. As noted previously, data showing the relative concentrations of topically applied drug in the retina and plasma does not appear to be often reported.

With respect to the anterior tissues of the eye (e.g., cornea), mecamylamine is also present in these tissues at high concentrations when compared to the concentration of mecamylamine in plasma and this difference can also be expressed, for example, as a ratio for the concentration of mecamylamine in corneal tissue (ng/g):concentration of mecamylamine in plasma (ng/mL). In general, the concentration of mecamylamine in corneal tissue is at least about 1000 times great than the concentration in plasma (i.e., [mecamylamine cornea (ng/g)]: [mecamylamine plasma (ng/mL) is at least 1000:1]). Similarly, the ratio of the concentration of mecamylamine in the aqueous humor versus the concentration of mecamylamine in plasma is also high, at least about 50:1 (i.e., [mecamylamine aqueous humor (ng/mL)]: [mecamylamine plasma (ng/mL) is at least 50:1]. Exemplary data for 3% (w/v) solutions of mecamylamine hydrochloride are provided below in Tables A and B, with additional details provided in the Examples.

TABLE A
Mecamylamine Distribution in Cornea compared to plasma
following topical ocular instillation of Mecamylamine

| | Concentration of Meca in Tissue/Plasma | | |
|---|---|---|---|
| | at 1 Hour (mean values ng/g or ng/mL ) | | |
| Test Article | Cornea | Plasma | Ratio |
| Polymer-free | 88,425 | 35 | 2,526:1 |
| Solution with 3% | | | |
| Meca single | | | |
| dose at 30 minutes | | | |
| Polymer-free | 36,000 | 18 | 2,000:1 |
| Solution with 3% | | | |
| Meca single | | | |
| dose at 1 hour | | | |
| Polymer-free | 24,817 | 15 | 1,654:1 |
| Solution with 3% | | | |
| Meca single dose | | | |
| at 1 hr after dosing | | | |
| Polymer-free | 56,517 | 43 | 1,314:1 |
| Solution with 3% | | | |
| Meca 6 doses in | | | |
| one day at 1 hr | | | |
| after last dose | | | |

TABLE B
Mecamylamine Distribution in Aqueous Humor compared to plasma
following topical ocular instillation of Mecamylamine

| | | Concentration of Meca in Aq. Humor at 1 | | |
|---|---|---|---|---|
| | | Hour (mean values ng/mL) | | |
| | | Aqueous | | |
| Study | Test Article | Humor | Plasma | Ratio |
| | Polymer-free | 8,678 | 35 | 248:1 |
| | Solution with 3% | | | |
| | Meca single | | | |
| | dose at 30 minutes | | | |
| | Polymer-free | 3,045 | 18 | 169:1 |
| | Solution with 3% | | | |
| | Meca single | | | |
| | dose at 1 hour | | | |
| | Polymer-free | 2,492 | 15 | 166:1 |
| | Solution with 3% | | | |
| | Meca single dose | | | |
| | at 1 hr after dosing | | | |
| | Polymer-free | 3,423 | 43 | 79:1 |
| | Solution with 3% | | | |
| | Meca 6 doses in | | | |
| | one day at 1 hr | | | |
| | after last dose | | | |

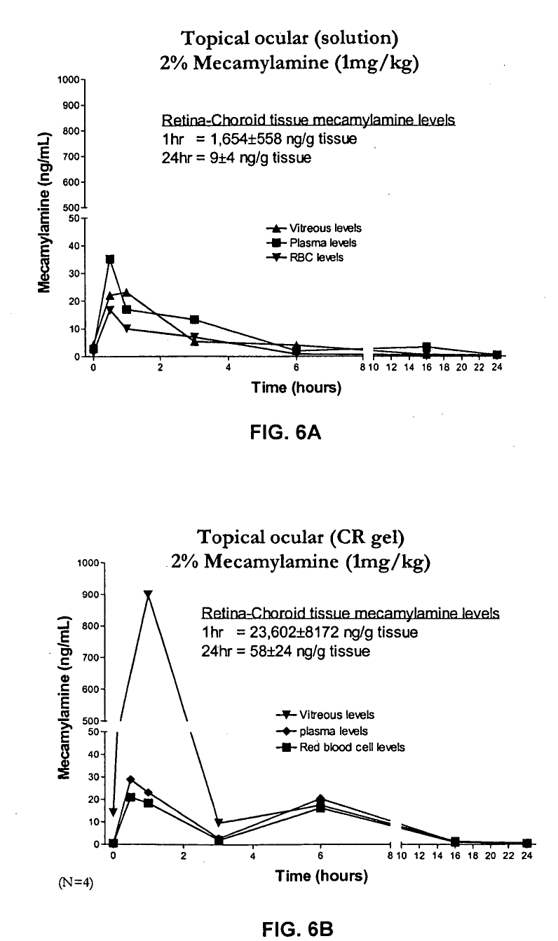

These surprising results illustrating the preferential absorption of mecamylamine into the tissues/fluids of the eye compared to the mecamylamine present in plasma can also be conveyed by simply measuring the mean maximum concentration of mecamylamine present in plasma when a therapeutically effective amount of mecamylamine (or a pharmaceutically acceptable salt thereof) is administered to the eye (e.g., for conditions affecting the posterior tissues of the eye (e.g., diabetic retinopathy, ROP, AMD, etc.) or for conditions affecting the anterior tissues of the eye (e.g., pterygium, corneal neovascularization, rubeosis iridis, etc.)). For example, FIGS. 6A and 6B clearly show that when mecamylamine hydrochloride is administered topically either as an aqueous solution free of polymer (FIG. 6A) or as an in situ gel-forming formulation (FIG. 6B), the mean maximum concentration of mecamylamine detected in plasma is surprisingly low, less than 50 ng/mL in each case. The comparison of the mean maximum concentration of mecamylamine for topical ocular administration with the mecamylamine mean maximum concentration data for the short (FIG. 1) and long (FIG. 2) intravenous administrations of mecamylamine hydrochloride is especially startling. For the short administration (FIG. 1), the mean maximum concentration of mecamylamine in plasma is in excess of 1500 ng/mL, while for the long infusion (FIG. 2), the mean maximum concentration of mecamylamine in plasma is greater than 500 ng/mL.

As noted previously, the side effects of systemic administration of mecamylamine include mydriasis (dilated pupil) and blurred vision. As these side effects are manifested in the eye, it would be expected that topical ocular application of mecamylamine (or a pharmaceutically acceptable salt thereof) would likely lead to a manifestation of these particular side effects. However, topical ocular application of mecamylamine hydrochloride to rabbits did not result in significant changes in pupil diameter or pupil diameter response to light, which suggests that topical ocular application of the mecamylamine hydrochloride to the rabbit eye is not inducing detectable levels of mydriasis, a quite surprising result. Thus, it is likely that topical ocular administration also avoids the side effect of blurred vision. Indeed, initial clinical trials in humans support the results of the animal studies with none of the subjects tested showing an increase in pupil diameter (no appearance of mydriasis) and none of the subjects tested reporting blurred vision (e.g., no change in best corrected visual acuity). Further, the initial clinical trials in humans, results from which are described in Examples 11 and 12, confirm that topical ocular administration of mecamylamine avoids or lessens the side effects (toxicity) described herein associated with systemic administration of mecamylamine (e.g., oral or transdermal administration that results in appreciable amounts of mecamylamine being present in the circulatory system), for example, constipation, urinary retention, postural hypotension, dry mouth, changes in pulse, changes in blood pressure, changes in ECG parameters, etc., and is also extremely well tolerated as an topical ocular formulation (e.g., no change in tear production, no reports of discomfort, no changes in intraocular pressure, no appearance of corneal erosion, no ulcers, no anterior chamber abnormalities, no conjunctival irritation/redness, no lens and/or retinal abnormalities, etc.), which many drugs are not. Thus clinical investigations in humans and non-human animal testing (e.g., rabbits, dogs) suggest that topical ocular mecamylamine formulations are well suited to the treatment of the conditions described herein, while also decreasing the pain, anxiety, and cost associated with the current standard of care (and thus also increasing patient compliance).

Additionally, in vitro studies demonstrated that mecamylamine inhibited VEGF-induced endothelial tube formation.

Pharmaceutical Formulations of Mecamylamine

Provided herein are pharmaceutical formulations of mecamylamine, or a pharmaceutically acceptable salt thereof, where the formulations are formulated for topical delivery to the eye.

As will be understood by those of skill in the art of ocular drug delivery, the terms “formulated for topical delivery to the eye,” “formulated for ocular topical administration,” “suitable for topical administration to the eye” and cognates thereof, generally refer to formulations (or components of the formulations) that can be tolerated by the individual to whom they are administered via the eye. In certain embodiments, the formulations so formulated do not cause undue tearing that may reduce the amount of mecamylamine being delivered to the posterior or anterior tissues below a therapeutically effective amount. From in vivo rabbit and dog studies, it appears that mecamylamine, and its HCl salt, are well tolerated in the ocular environment and do not cause undue irritation. In general, the formulations should also be sterile and free of pyrogens, irritants or other contamination and, where intended for administration to humans, meet all GMP (Good Manufacturing Process) and regulatory requirements. In some cases, topical ocular formulations will be isotonic, but this is not a requirement for formulations to be well tolerated by the eye and to meet regulatory requirements.

In certain embodiments, the pharmaceutical formulations may include mecamylamine, or a pharmaceutical acceptable salt thereof, and a carrier.

Mecamylamine, is also known as N-2,3,3-tetramethylbicyclo(2.2.1)heptan-2-amine, and marketed as INVERSINE® (indicated for the treatment of hypertension) and has the structure shown below. The pkₐ of mecamylamine is approximately pH 11.2. Early references describing the synthesis and characterization of mecamylamine include U.S. Pat. No. 2,831,027, Stone et al., (1962) J. Med. & Pharm. Chem. 5(4):665-690 and Stein et al., (1956). J. Am. Chem. Soc. 78:1514.

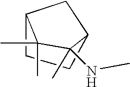

### N-2,3,3-tetramethylbicyclo[2.2.1]heptan-2-amine

In certain embodiments, mecamylamine may be incorporated in the formulations as the R- or S-isomer of mecamylamine, for example, substantially pure R-mecamylamine (e.g., free or substantially free of S-mecamylamine) or substantially pure S-mecamylamine (e.g., free or substantially free of R-mecamylamine). Preparation of substantially pure S-mecamylamine and R-mecamylamine has been described in the art, and various characteristics of the individual isomers have also been studied. See for example, Papke et al., (2001) J. Pharmacol. Exp. Therapeutics 297: 646-656; Pfister et al., (1962) J. Med. Pharm. Chem. 5(4): 665-690; U.S. Pat. App. Nos. 2002/0016371, 2002/0016370, 2004/004408, and U.S. Pat. No. 7,101,916, which are hereby incorporated by reference in their entirety. In some embodiments, the mecamylamine is substantially pure R-mecamylamine (or a pharmaceutically acceptable salt thereof). In other embodiments, the mecamylamine is substantially pure S-mecamylamine (or a pharmaceutically acceptable salt thereof).

In some embodiments, mecamylamine may be incorporated in the formulation as a pharmaceutically acceptable salt. Pharmaceutically acceptable salts are well known to those of skill in the art. Generally, pharmaceutically acceptable salts are those salts that retain substantially one or more of the desired pharmacological activities of the mecamylamine and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo(2.2.2)-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In certain embodiments where mecamylamine is incorporated as a pharmaceutically acceptable salt, the salt is the hydrochloride salt of mecamylamine. For example, mecamylamine hydrochloride, which is commercially available from Poli Industria Chimica, Milan, Italy. In particular embodiments, the carrier may include water. In some embodiments, the carrier may be an aqueous solution of saline, for example, water containing physiological concentrations of sodium, potassium, calcium, magnesium, and chloride at a physiological pH. In some embodiments, the carrier may be water and the formulation may further include NaCl. In some embodiments, the formulation may be isotonic. In some embodiments, the formulation may be hypotonic. In others, hypertonic. In some embodiments, the formulation is substantially free of polymers (e.g., gel-forming polymers, polymeric viscosity-enhancing agents, etc.). In some embodiments, the formulation is substantially free of viscosity-increasing agents (e.g., carboxymethylcellulose, polyanionic polymers, etc.). In some embodiments, the formulation is substantially free of gel-forming polymers. In some embodiments, the viscosity of the formulation is about the same as the viscosity of a saline solution containing the same concentration of mecamylamine (or a pharmaceutically acceptable salt thereof).

As will be understood by the skilled artisan, for formulations where the carrier includes a gel-forming polymer, in certain formulations the inclusion of salt(s), in particular saline solution, is contraindicated as inclusion of salt may either cause the solution to gel prior to topical ocular administration, as with certain in situ gel-forming polymers (e.g., gellan gel), or the inclusion of salts may inhibit the gelling properties of the gel-forming polymer. The skilled artisan will be able to select appropriate combinations based on the desired properties of the formulation and characteristics of gel-forming polymers known in the art.

Suitable aqueous saline solutions for use in the eye will be understood by those of skill in the art and may include, for example, solutions at a pH of from about pH 4.5 to about pH 8.0. In further variations of aqueous solutions (where water is included in the carrier), the pH of the formulation is between any of about 6 and about 8.0; between about 6 and about 7.5; between about 6 and about 7.0; between about 6.2 and about 8; between about 6.2 and about 7.5; between about 7 and about 8; between about 6.2 and about 7.2; between about 5.0 and about 8.0; between about 5 and about 7.5; between about 5.5 and about 8.0; between about 6.1 and about 7.7; between about 6.2 and about 7.6; between about 7.3 and about 7.4; about 6.0; about 7.1; about 6.2; about 7.3; about 6.4; about 6.5; about 6.6; about 6.7; about 6.8; about 6.9; about 7.0; about 7.1; about 7.2; about 7.3; about 7.4; about 7.5; about 7.6; or about 8.0. In some variations, the topical ocular mecamylamine formulation has a pH of about 6.0 to about 7.0. In some variations, the formulation has a pH of about 7.4. In particular variations, the formulation has a pH of about 6.2 to about 7.5.

In certain embodiments the concentration of the salt (e.g., NaCl) will be, for example, from about 0% to about 0.9% (w/v). For example, the concentration of salt may be from about 0.01 to about 0.9%, from about 0.02% to about 0.9%, from about 0.03% to about 9%, from about 0.05% to about 0.9% from about 0.07% to about 0.9%, from about 0.09% to about 0.9%, from about 0.1% to about 0.9% from about 0.2% to about 0.9%, from about 0.3% to about 0.9%, from about 0.4% to about 0.9% from about 0.5% to about 0.9%, from about 0.6% to about 0.9%, from about 0.7% to about 0.9%, from about 0.8% to about 0.9%, about 0.9%, about 0%, about 0.05%, about 0.01%, about 0.09%, about 0.1%, about 0.2%, about 0.3%, about 0.4%, about 0.5%, about 0.6%, about 0.7%, or about 0.8%. In certain embodiments, the aqueous saline solution will be isotonic (e.g., NaCl concentration of about 0.9% NaCl (w/v)). In certain embodiments, the aqueous solution will contain a NaCl concentration of about 0.5%, about 0.7%, about 0.8%, about 0.85, or about 0.75%. As will be appreciated the skilled artisan, depending on the concentrations of other components, for example where mecamylamine hydrochloride or other salts of mecamylamine are used, the concentration of NaCl or other salt needed to achieve an formulation suitable for administration to the eye may vary.

In some embodiments where the carrier includes water, the ratio of the concentration of mecamylamine present in the choroidal and retinal tissue when topically administered to a rabbit eye, measured in units of ng/g, to the concentration of mecamylamine in plasma measured in units of ng/mL ([ng/g mecamylamine choroidal+retinal tissue]: [ng/mL plasma]) is at least about 20:1, at least about 25:1, at least about 30:1, at least about 35:1, at least about 40:1, at least about 45:1, at least about 50:1, at least about 55:1, at least about 60:1, at least about 70:1, at least about 80:1, at least about 100:1, at least about 150:1, at least about 200:1, at least about 250:1, at least about 300:1, at least about 350:1, at least about 375:1, at least about 400:1, at least about 425:1, at least about 450:1, at least about 475:1, at least about 500:1, at least about 550:1, at least about 600:1, at least about 650:1, at least about 700:1, at least about 750:1, at least about 800:1, at least about 850:1, at least about 900:1, at least about 950:1, at least about 1000:1, at least about 1025:1, at least about 1050:1, at least about 1100:1, at least about 1200:1, at least about 1300:1, at least about 1500:1, at least about 1700:1, at least about 2000:1 or at least 2500:1. In some embodiments, the ratio is from about 20:1 to about at 2500:1, from about 20:1 to about 2000:1, from about 20:1 to about 1500:1, from about 20:1 to about 1000:1, from about 20:1 to about 1500:1, from about 20:1 to about 2000:1, from about 20:1 to about 800:1, from about 20:1 to about 500:1, from about 20:1 to about 300:1, from about 20:1 to about 200:1, from about 20:1 to about 100:1, from about 30:1 to about at 2500:1, from about 30:1 to about 3000:1, from about 30:1 to about 1500:1, from about 30:1 to about 1000:1, from about 30:1 to about 800:1, from about 30:1 to about 500:1, about 30:1 to about 300:1, from about 30:1 to about 300:1, from about 30:1 to about 100:1, from about 40:1 to about at 2500:1, from about 40:1 to about 4000:1, at least about 40:1 to about 2500:1, from about 40:1 to about 1500:1, from about 40:1 to about 1000:1, from about 40:1 to about 2000:1, from about 40:1 to about 800:1, from about 40:1 to about 500:1, about 40:1 to about 300:1, from about 40:1 to about 400:1, from about 40:1 to about 100:1, from about 300:1 to about at 2500:1, from about 300:1 to about 2000:1, from about 300:1 to about 1500:1, from about 3.00:1 to about 1000:1, from about 300:1 to about 800:1, from about 350:1 to about at 2500:1, from about 350:1 to about 2000:1, from about 350:1 to about 1500:1, from about 350:1 to about 1000:1, from about 350:1 to about 800:1, from about 400:1 to about at 2500:1, from about 400:1 to about 2000:1, from about 400:1 to about 1500:1, from about 400:1 to about 1000:1, from about 400:1 to about 800:1, from about 450:1 to about at 2500:1, from about 450:1 to about 2000:1, from about 450:1 to about 1500:1, from about 450:1 to about 1000:1, from about 450:1 to about 800:1, from about 500:1 to about at 2500:1, from about 500:1 to about 2000:1, from about 500:1 to about 1500:1, from about 500:1 to about 1000:1, or from about 500:1 to about 800:1. In particular embodiments, the ratio is at least about 300:1, at least about 350:1, at least about 450:1, at least about 500:1, at least about 1200:1, from about 300:1 to about 1000:1, from about 300:1 to about 2000:1, from about 350:1 to about 1000:1, from about 350:1 to about 2000:1, from about 450:1 to about 1000:1, from about 450:1 to about 1100:1, from about 450:1 to about 1200:1, from about 450 to about 2000:1, from about 500:1 to about 1000:1, from about 500:1 to about 1200:1, or about 500:1 to about 2000:1. In some of these embodiments, the formulation further includes a viscosity-increasing agent (e.g., hypromellose, etc.). In some embodiments, the formulation is substantially free of polymers (e.g., gel-forming polymers, polymeric viscosity-enhancing agents, etc.). In some embodiments, the formulation is substantially free of viscosity-increasing agents (e.g., carboxymethylcellulose, polyanionic polymers, etc.). In some embodiments, the formulation is substantially free of gel-forming polymers. In some embodiments, the viscosity of the formulation is about the same as the viscosity of a saline solution containing the same concentration of mecamylamine (or a pharmaceutically acceptable salt thereof).

In some embodiments where the carrier includes water, the ratio of the concentration of mecamylamine present in the corneal tissue when topically administered to a rabbit eye, measured in units of ng/g, to the concentration of mecamylamine in plasma measured in units of ng/mL ([ng/g mecamylamine corneal tissue]: [ng/mL plasma]) is at least about 1000:1. In some embodiments, the ratio of concentrations is at least about 100:1, at least about 200:1, at least about 300:1, at least about 400:1, at least about 500:1 at least, at least about 600:1, at least about 700:1, at least about 800:1, at least about 850:1, at least about 900:1, at least about 950:1, at least about 1000:1, at least about 1025:1, at least about 1050:1, at least about 1100:1, at least about 1200:1, at least about 1300:1, at least about 1500:1, at least about 1700:1, at least about 2000:1 or at least 2500:1. In some embodiments, the ratio is from at least about 100:1 to about 4000:1, from at least about 100:1 to about 3000:1, from at least about 100:1 to about 2500:1, from at least about 800:1 to about 4000:1, from at least about 800:1 to about 3000:1, from at least about 800:1 to about 2500:1, from at least about 900:1 to about 4000:1, from at least about 900:1 to about 3000:1, from at least about 1000:1 to about 4000:1, from at least about 1000:1 to about 3000:1, from at least about 1000:1 to about 2500:1, from at least about 1000:1 to about 2000:1. In certain embodiments, the ratio is at least about 850:1, at least about 900:1, at least about 1000:1 at least about 1200:1. In some of these embodiments, the formulation further includes a viscosity-increasing agent (e.g., hypromellose, etc.). In other embodiments, the formulation is substantially free of polymers (e.g., gel-forming polymers, viscosity-enhancing agents, etc.). In some embodiments, the formulation is substantially free of viscosity-increasing agents (e.g., carboxymethylcellulose, polyanionic polymers, etc.). In some embodiments, the formulation is substantially free of gel-forming polymers. In some embodiments, the viscosity of the formulation is about the same as the viscosity of a saline solution containing the same concentration of mecamylamine (or a pharmaceutically acceptable salt thereof).

In some embodiments, where the formulation is substantially free of viscosity-increasing agents, the formulation may be substantially free of viscosity-increasing agents such as, but not limited to polyanionic polymers, water soluble cellulose derivatives (e.g., hypromellose (also known as HPMC, hydroxypropylmethyl cellulose, and hydroxypropylcellulose), hydroxyethylcellulose, carboxmethylcellulose, etc.), polyvinyl alcohol, polyvinyl pyrrolidone, chondroitin sulfate, hyaluronic acid, soluble starches, etc. In some variations, the formulation does not incorporate a hydrogel or other retention agent (e.g., such as those disclosed in U.S. Pat. Pub. No. 2005/0255144 (incorporated by reference herein in its entirety)), e.g., where they hydrogel may include, hydrogels incorporating homopolymers; copolymers (e.g., tetrapolymers of hydroxymethylmethacrylate, ethylene glycol, dimethylmethacrylate, and methacrylic acid), copolymers of trimethylene carbonate and polyglycolicacid, polyglactin 910, glyconate, poly-p-dioxanone, polyglycolic acid, polyglycolic acid felt, poly-4-hydroxybutyrate, a combination of poly(L-lactide) and poly(L-lactide-co-glycolide), glycol methacrylate, poly-DL-lactide, or Primacryl); composites of oxidized regenerated cellulose, polypropylene, and polydioxanone or a composite of polypropylene and poligelcaprone; etc. In some variations, the formulations do not include one or more of polyvinyl alcohol, hydroxypropyl methylcellulose, polyethylene glycol 400 castor oil emulsion, carboxymethylcellulose sodium, propylene glycol, hydroxypropyl guar, carboxymethylcelluose sodium, white petrolatum, mineral oil, dextran 70, glycerin, hypromellose, flaxseed oil, fish oils, omega 3 and omega 6 fatty acids, lutein, or primrose oil. In some variations, the formulations do not include one or more of the carriers described in U.S. Pat. No. 4,888,354 (incorporated by reference herein in its entirety), e.g., such as one or more of oleic acid, ethanol, isopropanol, glycerol monooleate, glycerol diooleate, methyl laurate, propylene glycol, propanol or dimethyl sulfoxide. In some variations, the formulations are substantially free of glycerol diooleate and isopropanol.

In some embodiments where the carrier includes water, the ratio of the concentration of mecamylamine present in the aqueous humor when topically administered to a rabbit eye, measured in units of ng/mL, to the concentration of mecamylamine in plasma measured in units of ng/mL ([ng/mL mecamylamine aqueous humor]: [ng/mL plasma]) is at least about 40:1, at least about 45:1, at least about 50:1, at least about 55:1, at least about 60:1, at least about 70:1, at least about 80:1, at least about 100:1, at least about 150:1, at least about 200:1, or at least about 250:1. In some embodiments, the ratio is from about 40:1 to about at 2500:1, from about 40:1 to about 4000:1, from about 40:1 to about 2000:1, from about 40:1 to about 1500:1, from about 40:1 to about 1000:1, from about 40:1 to about 800:1, from about 40:1 to about 500:1, about 40:1 to about 300:1, from about 40:1 to about 400:1, or from about 40:1 to about 100:1. In particular embodiments, the ratio is at least about 50:1. In some of these embodiments, the formulation further includes a viscosity-increasing agent (e.g., hypromellose, etc.). In other embodiments, the formulation is substantially free of polymers (e.g., gel-forming polymers, viscosity-enhancing agents, etc.). In some embodiments, the formulation is substantially free of viscosity-increasing agents (e.g., carboxymethylcellulose, polyanionic polymers, etc.). In some embodiments, the formulation is substantially free of gel-forming polymers. In some embodiments, the viscosity of the formulation is about the same as the viscosity of a saline solution containing the same concentration of mecamylamine (or a pharmaceutically acceptable salt thereof).

As noted previously, quite surprisingly the topical ocular administration of an amount of mecamylamine (or a pharmaceutically acceptable salt thereof) effective to reduce abnormal angiogenesis and/or neovascularization of the tissues of the eye (either anterior or posterior) results in extremely low levels of mecamylamine in plasma. For example, the mean maximum concentration of mecamylamine detected is less than about 70 ng/mL. These low maximum concentrations of mecamylamine are observed in both single dose and multi-dose (e.g., multiple doses per day or multiple doses over a number of days) studies for rabbits administered mecamylamine (or a pharmaceutically acceptable salt thereof) in a topical ocular form. Thus, for example, the mean maximum concentration of mecamylamine detected will be less than about 65 ng/mL, less than about 60 ng/mL, less than about 55 ng/mL, less than about 50 ng/mL, less than about 45 ng/mL, less than about 40 ng/mL, less than about 20 ng/mL, less than about 15 ng/mL, less than about 10 ng/mL, or less than about 5 ng/mL. In particular embodiments, where the mecamylamine is administered in a single dose per day, the mean maximum concentration of plasma may be less than about 25 ng/mL. For example, less than about 20 ng/mL, less than about 15 ng/mL, less than about 10 ng/mL, or less than about 5 ng/mL.

Alternatively, the amount of mecamylamine appearing in plasma can be measured as the total concentration of mecamylamine as measured as the area under the curve (AUC) for the concentration of mecamylamine after administration. As with other measures of mecamylamine in plasma, the mean total concentration of mecamylamine in plasma over a given population of subjects is surprisingly low. For example, less than about 85 ng/mL-hr. In some embodiments, the amount of mecamylamine as measured by this method is less than about 100 ng/mL-hr, less than about 90 ng/mL-hr, less than about 80 ng/mL-hr, less than about 75 ng/mL-hr, less than about 70 ng/mL-hr, less than about 65 ng/mL-hr, less than about 60 ng/mL-hr, less than about 50 ng/mL-hr or less than about 45 ng/mL-hr.

Exemplary values for mecamylamine AUC concentration for 3% (w/v) formulations of mecamylamine hydrochloride administered as single 50 μl doses to each eye are shown below. The values calculated for the AUC for the tissues and plasma may also be used to calculate ratios between the cornea, choroid/retina and aqueous humor versus plasma, as also shown below in Table C. Data was obtained as described in Example 8.

TABLE C

| | Gellan formulation | Solution | HPMC |
|---|---|---|---|
| Plasma AUC | 41 | 83 | 61 |
| (ng/mL*hr) | | | |
| Retina/Choroid AUC | 15145 | 7410 | 17804 |
| (ng/mg*hr) | | | |
| Ratio Retina/ | 369:1 | 89:1 | 291:1 |
| Choroid:Plasma | | | |
| Cornea AUC | 111,725 | 111,563 | 113,938 |
| (ng/mg*hr) | | | |
| Ratio Cornea:Plasma | 2,725:1 | 1,344:1 | 1,868:1 |
| Aqueous Humor AUC | 8,690 | 7,501 | 7,355 |
| (ng/mL*hr) | | | |
| Ratio Aqu H:plasma | 212 | 90 | 120 |

When the AUC value is viewed as a ratio, the ratio of the AUC of the retina/choroid:AUC plasma is at least about 50:1, at least about 55:1, at least about 60:1, at least about 70:1, at least about 75:1, at least about 80:1, at least about 90:1, at least about 100:1, at least about 150:1, at least about 200:1, at least about 250:1, at least about 300:1, or at least about 350:1. In some embodiments, the ratio of the AUC of the retina/choroid:AUC plasma is at least about 80:1. When the AUC value is viewed as a ratio, the ratio of the AUC of the cornea:AUC plasma is at least about 100:1, at least about 500:1, at least about 600:1, 800:1, at least about 900:1, at least about 1000:1, at least about 1500:1, at least about 2000:1, or at least about 2500:1. In certain embodiments, the ratio of the AUC of the cornea:AUC plasma is at least about 1000:1. When the AUC value is viewed as a ratio, the ratio of the AUC of the aqueous humor:AUC plasma is at least about 50:1, at least about 60:1, at least about 80:1, at least about 90:1, at least about 100:1, at least about 150:1, or at least about 200:1. In some embodiments, the ratio of the AUC of the aqueous humor:AUC plasma is at least about 90:1.

In particular embodiments, the pharmaceutical formulations may include mecamylamine, or a pharmaceutically acceptable salt thereof, water and gel-forming polymer, wherein the gel-forming polymer is characterized such that when the formulation is topically administered to a rabbit eye, the ratio of the concentration of mecamylamine present in the choroidal and retinal tissue, measured in units of ng/g, to the concentration of mecamylamine in plasma measured in units of ng/mL ([ng/g mecamylamine choroidal+retinal tissue]: [ng/mL plasma]) is at least about 300:1.

As used herein, the term “topically administered to a rabbit eye,” and cognates thereof, refers to the administration of a particular mecamylamine formulation, including formulations of pharmaceutically acceptable salts of mecamylamine, by applying 100 μL of the mecamylamine formulation to the cornea of each eye (2 drops of 50 μL each), wherein at the time of application the bottom eyelid is separated from the surface of the eye to make a pocket to ensure the entire dose is retained in contact with the eye. When the concentration of mecamylamine in plasma and in the retinal/choroidal tissue is to be measured in connection with the calculation of the ratio of the mecamylamine concentration in the retina/choroid versus the concentration of mecamylamine in plasma, the concentration of each is determined 1 hour after application of the formulation. The amount of mecamylamine present in plasma and the retina/choroid is determined by LC/MS/MS using internal standards of dextromethorphan and diphenhydramine and a standard of known concentration of mecamylamine. An exemplary method of administration of the topical formulation of mecamylamine is provided in Example 5. An exemplary LC/MS/MS method for determining the ratios described herein is given in detail in Example 6. When the mean maximum concentration of mecamylamine in plasma is to be determined, the concentration is again determined as described above and in Example 6. The term “mean maximum concentration of mecamylamine,” and its cognates, as used herein refers to the mean maximum concentration of mecamylamine measured in plasma when monitored over a 6 hour time period after administration of the formulation. When the mean total concentration of mecamylamine in plasma is to be determined, the concentration is again determined as described above and in Example 6. The term “total concentration of mecamylamine in plasma measured as the area under the curve,” (AUC) and cognates thereof, refers to summation of the areas under plot of the drug plasma versus time using a simple trapezoidal method with drug plasma time points taken to include 1, 3 and 6 hours after topical administration. Determination of AUC for corneal tissue, retinal/choroid tissue and aqueous humor is determined similarly. When the concentration of mecamylamine in plasma and in the corneal tissue is to be measured in connection with the calculation of the ratio of the mecamylamine concentration in the cornea (or aqueous humor) versus the concentration of mecamylamine in plasma, the concentration of each is determined 1 hour after application of 50 μl of the formulation. The amount of mecamylamine present in plasma and the cornea (or aqueous humor) is determined by LC/MS/MS using internal standards of dextromethorphan and diphenhydramine and a standard of known concentration of mecamylamine. An exemplary method of administration of the topical formulation of mecamylamine is provided in Example 5. An exemplary LC/MS/MS method for determining the ratios described herein is given in detail in Example 6.

In some embodiments, the formulation is a gel prior to topical ocular administration.

In other embodiments, the formulation forms a gel in situ upon topical ocular administration. Examples of in situ gel-forming formulations are formulations that form gels in response to a change in tonicity (e.g., GELRITE® (a gellan gum), temperature, salt concentration, etc.) Examples of formulations including in situ gel-forming polymers are described in, for example, U.S. Pat. Nos. 6,174,524; 4,861,760.

As used herein, the terms “topical ocular administration” or “topically administered,” and cognates of these terms, refer to contacting the surface of the eye with the formulation. Contacting may be accomplished by methods known to those of skill in the art, including, but not limited to, eye drops, application of gel formulations, applications of gels, application of films, etc.

In particular embodiments, the gel-forming polymer may be, for example, a polysaccharide. In certain embodiments, the polysaccharide is gellan gum. Gellan gum refers to a heteropolysaccharide elaborated by the bacterium Pseudomonas elodea, though the name “gellan gum” is more commonly used in the field. Gellan gum, in particular the formulation GELRITE® is described in detail in U.S. Pat. No. 4,861,760 (hereby incorporated by reference in its entirety), in particular in its use in formulation of timolol. GELRITE®, a low acetyl clarified grade of gellan gum, is commercially available from Merck & Co (Rahway, N.J.) and gellan gum can be commercially obtained from, among others CPKelco (Atlanta, Ga.). The preparation of polysaccharides such as gellan gum is described in, for example, U.S. Pat. Nos. 4,326,053 and 4,326,052, which are hereby incorporated by reference in their entirety.

In certain embodiments, the gel-forming polymer is present at a concentration of from about 0.03% to about 2% (w/v). In some embodiments, the gel-forming polymer is present at a concentration from about 0.03% to about 1.75%; from about 0.03% to about 1.5%, from about 0.03% to about 1.25%, from about 0.03% to about 1%, from about 0.03% to about 0.9%, from about 0.03% to about 0.8%, from about 0.03% to about 0.7%, from about 0.03% to about 0.6%, from about 0.03% to about 0.5%, from about 0.05% to about 2%, from about 0.05% to about 1.75%; from about 0.05% to about 1.5%, from about 0.05% to about 1.25%, from about 0.05% to about 1%, from about 0.05% to about 0.9%, from about 0.05% to about 0.8%, from about 0.05% to about 0.7%, from about 0.05% to about 0.6%, from about 0.05% to about 0.5%, from about 0.1% to about 2%, from about 0.1% to about 1.75%; from about 0.1% to about 1.5%, from about 0.1% to about 1.25%, from about 0.1% to about 1%, from about 0.1% to about 0.9%, from about 0.1% to about 0.8%, from about 0.1% to about 0.7%, from about 0.1% to about 0.6%, from about 0.1% to about 0.5%, from about 0.2% to about 2%, from about 0.2% to about 1.75%; from about 0.2% to about 1.5%, from about 0.2% to about 1.25%, from about 0.2% to about 1%, from about 0.2% to about 0.9%, from about 0.2% to about 0.8%, from about 0.2% to about 0.7%, from about 0.2% to, about 0.6%, from about 0.2% to about 0.5%, or from about 0.5% to about 1.5%. In some embodiments, the concentration of gel-forming polymer is about 0.1%, about 0.2%, about 0.4%, about 0.6%, about 0.8%, about 1%.

In particular embodiments, the gel-forming polymer is gellan gum at a concentration of from about 0.05% to about 2% (w/v), from about 0.1% to about 2% (w/v), from about 0.1% to about 1% (w/v), from about 0.05% to about 1% (w/v) or from about 0.1% to about 0.6% (w/v). In some embodiments, the concentration of gellan gum is about 0.1%, about 0.2%, about 0.4%, about 0.6%, about 0.8%, about 1%.

In some embodiments where a gel-forming polymer is present in the formulation, the ratio of the concentration of mecamylamine present in the choroidal and retinal tissue when topically administered to a rabbit eye, measured in units of ng/g, to the concentration of mecamylamine in plasma measured in units of ng/mL ([ng/g mecamylamine choroidal+retinal tissue]: [ng/mL plasma]) is at least about 350:1, at least about 375:1, at least about 400:1, at least about 425:1, at least about 450:1, at least about 475:1, at least about 500:1, at least about 550:1, at least about 600:1, at least about 650:1, at least about 700:1, at least about 750:1, at least about 800:1, at least about 850:1, at least about 900:1, at least about 950:1, at least about 1000:1, at least about 1025:1, at least about 1050:1, at least about 1100:1, at least about 1200:1, at least about 1300:1, at least about 1500:1, at least about 1700:1, at least about 2000:1 or at least 2500:1. In some embodiments, the ratio is from about 300:1 to about at 2500:1, from about 300:1 to about 2000:1, from about 300:1 to about 1500:1, from about 300:1 to about 1000:1, from about 300:1 to about 800:1, from about 350:1 to about at 2500:1, from about 350:1 to about 2000:1, from about 350:1 to about 1500:1, from about 350:1 to about 1000:1, from about 350:1 to about 800:1, from about 400:1 to about at 2500:1, from about 400:1 to about 2000:1, from about 400:1 to about 1500:1, from about 400:1 to about 1000:1, from about 400:1 to about 800:1, from about 450:1 to about at 2500:1, from about 450:1 to about 2000:1, from about 450:1 to about 1500:1, from about 450:1 to about 1000:1, from about 450:1 to about 800:1, from about 500:1 to about at 2500:1, from about 500:1 to about 2000:1, from about 500:1 to about 1500:1, from about 500:1 to about 1000:1, or from about 500:1 to about 800:1. In particular embodiments, the ratio is at least about 300:1, at least about 350:1, at least about 450:1, at least about 500:1, at least about 1200:1, from about 300:1 to about 1000:1, from about 300:1 to about 2000:1, from about 350:1 to about 1000:1, from about 350:1 to about 2000:1, from about 450:1 to about 1000:1, from about 450:1 to about 1100:1, from about 450:1 to about 1200:1, from about 450 to about 2000:1, from about 500:1 to about 1000:1, from about 500:1 to about 1200:1, or about 500:1 to about 2000:1.

In some embodiments, the mecamylamine, or pharmaceutically acceptable salt thereof, may be present at a concentration of from about 0.001% to about 6% (w/v). In certain embodiments, the mecamylamine, or pharmaceutically acceptable salt thereof, may be present at concentration (w/v) of from about 0.001% to about 5%, from about 0.005% to about 6%, from about 0.005% to about 5%, from about 0.01% to about 6%, from about 0.01% to about 5%, from about 0.01% to about 4%, from about 0.01% to about 3%, from about 0.01% to about 2%, from about 0.01% to about 1%, from about 0.001% to about 4%, from about 0.001% to about 3%, from about 0.001% to about 2%, from about 0.001% to about 1%, from about 0.03% to about 4%; from about 0.03% to about 3%, from about 0.03% to about 2%, from about 0.03% to about 1%, from about 0.03% to about 0.5%, from about 0.03% to about 0.2%, from about 0.03% to about 0.1%, from about 0.1% to about 6%, from about 0.1% to about 5%, from about 0.1% to about 4%, from about 0.1% to about 3%, from about 0.1% to about 2%, from about 0.1% to about 1%, from about 0.3% to about 6%, from about 0.3% to about 5%, from about 0.3% to about 4%, from about 0.3% to about 3%, from about 0.3% to about 2%, from about 0.3% to about 1%, from about 0.5% to about 6%, from about 0.5% to about 5%, from about 0.5% to about 4%, from about 0.5% to about 3%, from about 0.5% to about 2%, from about 0.5% to about 1%, from about 1% to about 6%, from about 1% to about 5%, from about 1% to about 4%, from about 1% to about 3%, or from about 1% to about 2%. In some embodiments, the mecamylamine, or pharmaceutically acceptable salt thereof, may be present at a concentration of about 0.01%, about 0.02%, about 0.03%, about 0.04%, about 0.05%, about 0.8%, about 0.9%, about 0.1%, about 0.2%, about 0.3%, about 0.4%, about 0.5%, about 0.8%, about 1%, about 1.2%, about 1.5%, about 2%, about 2.5%, about 3%, about 3.5%, about 4%, about 5%, about 6% (w/v).

In particular embodiments, the mecamylamine, or pharmaceutically acceptable salt thereof, is present at a concentration (w/v) of, for example, from about 0.001% to about 6%, from about 0.001% to about 5%, from about 0.02% to about 2%, from about 0.02% to about 1%, from about 0.05% to about 2%, from about 0.05% to about 1%, from about 0.1 to about 5%, or from about 0.1 to about 3% and the gel-forming polymer is gellan gum at a concentration of from about 0.05% to about 2% (w/v), from about 0.1% to about 2% (w/v), from about 0.1% to about 1% (w/v), from about 0.05% to about 1% (w/v) or from about 0.1% to about 0.6% (w/v). In certain embodiments, the mecamylamine is present as a pharmaceutically acceptable salt. In some embodiments, the pharmaceutically acceptable salt is mecamylamine hydrochloride.

Additional Components

In some embodiments of the formulations, the formulation may include additional components such as one or more preservatives, one or more surfactants, or one or more pharmaceutical agents.

In particular embodiments, the formulation may include additional components such as one or more preservatives, one or more surfactants, one or more tonicity agents, one or more buffering agents, one or more chelating agents, one or more viscosity-increasing agents, one or more salts, or one or more pharmaceutical agents. In certain of these embodiments, the formulation may include (in addition to mecamylamine (or a pharmaceutically acceptable salt thereof) and carrier): one or more preservatives, one or more buffering agents (e.g., one, two, three, etc.), one or more chelating agents, and one or more salts. In some embodiments, the formulation may include (in addition to mecamylamine (or a pharmaceutically acceptable salt thereof) and carrier): one or more preservatives, one or more tonicity agents, one or more buffering agents, one or more chelating agents, and one or more viscosity-increasing agents.

As used herein, the term “pharmaceutical agent” or “additional pharmaceutical agent,” and cognates of these terms, are intended to refer to agents other than mecamylamine, or pharmaceutically acceptable salts thereof, e.g., drugs which are administered to elicit a therapeutic effect. The pharmaceutical agent(s) may be directed to a therapeutic effect related to the condition that the mecamylamine formulation is intended to treat or prevent, e.g., conditions mediated by neovascularization (e.g., retinal neovascularization, choroidal neovascularization), abnormal angiogenesis, or combinations thereof, of posterior tissues of the eye (e.g., proliferative retinopathies)); conditions mediated by neovascularization (e.g., corneal neovascularization, post-corneal transplant neovascularization, etc.), abnormal angiogenesis, or combinations thereof, of anterior tissues of the eye (e.g., pterygium, rubeosis iridis, neovascular glaucoma, etc.) or, the pharmaceutical agent may be intended to treat a symptom of the underlying condition or to further reduce the appearance or severity of side effects related to mecamylamine administration, although these are likely to occur in few individuals.

In some embodiments, the pharmaceutical agent(s) may be an nAChR antagonist, anti-inflammatory agent (e.g., NSAID, etc.), VEGF antagonist, VEGF, (e.g., VEGF TRAP, etc.), tyrosine kinase inhibitor, prostaglandin receptor antagonist, agent used in the treatment of glaucoma, or an agent to lower intra-ocular pressure. Selection of appropriate pharmaceutical agent(s) for use in the formulations and methods described herein will depend upon the condition to be treated, as will be appreciated by the skilled artisan. Exemplary pharmaceutical agents are described in greater detail below.

In certain embodiments, the pharmaceutical agent may be an antagonist of the nicotinic acetylcholine receptor (nAChR). Examples of nAChR antagonists are known in the art and include, for example, hexamethonium, dihydro-beta-erythroidine, d-tubocurarine, pempidine, chlorisondamine, erysodine, trimethaphan camsylate, pentolinium, bungarotoxin, succinylcholine, tetraethylammonium, trimethaphan, chlorisondamine, trimethidinium, etc. See, for example, Suñer et al, (2004) ibid. In some embodiments, the nAChR antagonist is hexamethonium.

In some embodiments, the pharmaceutical agent(s) may include one or more pharmaceutical agents shown to be effective in the treatment of the conditions described herein. For example, VEGF antagonists (e.g., anti-VEGF (vascular endothelial growth factor) antibodies or fragments thereof, VEGF apatamers (e.g., pegaptanib sodium). In certain embodiments, the anti-VEGF antibodies are monoclonal antibodies. Exemplary anti-VEGF antibodies include, but are not limited to, bevacizumab and ranibizumab (tradenames AVASTIN® and LUCENTIS®, respectively, under development by Genentech, Inc., South San Francisco, Calif.). Pharmaceutical agents may also include the Vascular Endothelial Growth Factor (VEGF) receptor antagonist pegaptanib (an aptamer) (MACUGEN®; Pfizer).

In some embodiments, the pharmaceutical agent(s) may be a tyrosine kinase inhibitor.

In some embodiments, the pharmaceutical agent is a VEGF scavenger. In some embodiments the VEGF scavenger is VEGF TRAP.

In some variations, the pharmaceutical agent is a VEGF scavenger, VEGF antagonist, or tyrosine kinase inhibitor.

In some embodiments, the pharmaceutical agent(s) may be an agent for treatment of glaucoma (e.g., dichlorphenamide, carbochol, demacarium bromide, etc.) or an agent for the lowering of intro-ocular pressure (e.g., steroids).

In some embodiments, the pharmaceutical agent(s) may be a non-steroidal anti-inflammatory drug (NSAID). Numerous NSAIDs are well known to the skilled artisan and can be selected based on the condition to be treated as well as the general health of the individual to be treated. Exemplary classes of NSAIDs include, but are not limited to, e.g., salicylates (e.g., aspirin, methyl salicylate, etc.), arylalkanoic acids (e.g., diclofenac, sulindac, etc.), 2-arylpropionic acids (profens (e.g. ibuprofen, ketoprofen, naproxen, etc.), N-arylanthranilic acids (fenamic acids) (e.g., mefenamic acid, etc.), pyrazolidine derivatives (e.g., oxyphenylbutazone, phenylbutazone, etc.), oxicams (e.g., piroxicam, meloxicam, etc.), selective COX-2 inhibitors (e.g., coxibs (e.g., celecoxib, parecoxib, etc.), sulphonanilides (e.g., nimesulide, etc.), and selective COX-3 inhibitors.

In some embodiments, the pharmaceutical agent(s) may be a prostaglandin receptor antagonist.

In particular embodiments, where the carrier is water, the formulation may be substantially free of polymers (e.g., does not contain a polymeric viscosity-increasing agent, gel-forming polymer, etc.). In some embodiments, the formulation is substantially free of viscosity-increasing agent(s) (e.g., carboxymethylcellulose, polyanionic polymers, etc.). In some embodiments, the viscosity of the formulation is about the same as the viscosity of a saline solution containing the same concentration of mecamylamine (or a pharmaceutically acceptable salt thereof). In some embodiments, the formulation is substantially free of gel-forming polymers. In certain embodiments, where the carrier is water, the formulation may additionally include one or more chelating agents (e.g., edetate disodium (EDTA), one or more preservatives (e.g., benzalkonium chloride, benzethonium chloride, chlorhexidine, chlorobutanol, methylparaben, phenylethyl alcohol, propylparaben, thimerosal, phenylmercuric nitrate, phenylmercuric borate, phenylmercuric acetate, or combinations of two or more of the foregoing), salt (e.g., NaCl) and one or more buffering agents (e.g., one or more phosphate buffers (e.g., dibasic sodium phosphate, monobasic sodium phosphate, combinations thereof, etc.), citrate buffers, maleate buffers, borate buffers, and combination of two or more of the foregoing.).

In particular embodiments, the chelating agent is edetate disodium, the preservative is benzalkonium chloride, the salt is NaCl, and the buffering agents are dibasic sodium phosphate and monobasic sodium phosphate. In certain of these embodiments, the formulation is substantially free of polymer. In some embodiments, the formulation is substantially as described in Table 16. In some embodiments, the formulation is substantially free of substantially viscosity-increasing agent(s) (e.g., carboxymethylcellulose, polyanionic polymers, etc.). In some embodiments, the viscosity of the formulation is about the same as the viscosity of a saline solution containing the same concentration of mecamylamine (or a pharmaceutically acceptable salt thereof). In some of these embodiments, the concentration of mecamylamine (or a pharmaceutically acceptable salt thereof) if from about 0.02% to about 3%, from about 0.02% to about 2%, from about 0.02% to about 1% (w/v). In certain embodiments, the concentration of mecamylamine (or a pharmaceutically acceptable salt thereof), is about 0.01%, about 0.02%, about 0.03%, about 0.05%, about 0.07%, about 0.1%, about 0.3%, about 0.4%, about 0.5%, about 0.6%, about 0.8% or about 1% (w/v).

In certain embodiments, where the carrier includes water, a viscosity-increasing agent may also be included in the formulation. The skilled artisan will be familiar with viscosity-increasing agents that are suitable for use in the eye (e.g., water-soluble cellulose derivatives (e.g., hypromellose (also known as HPMC, hydroxypropylmethyl cellulose, and hydroxypropylcellulose), hydroxyethylcellulose, carboxmethylcellulose, etc.), polyvinyl alcohol, polyvinyl pyrrolidone, chondroitin sulfate, hyaluronic acid, and soluble starches. It is intended that when viscosity-increasing agents are used, they are not included in high enough concentrations such that the formulation would form a gel prior to or after administration (e.g., wherein the concentration of the viscosity-increasing agent is not sufficient to induce gel formation).

While exact concentrations of viscosity-increasing agents will depend upon the selection and concentration of other components in the formulation as well as the particular viscosity-increasing agent(s) selected, in general, viscosity-increasing agents may be present in a concentration such that the viscosity of the resulting solution is less than about 1000 centiopoise. In certain embodiments, the viscosity of the formulation is less than about 900, less than about 800, less than about 700, less than about 600, less than about 500, less than about 400, less than about 300, less than about 200, less than about 150, less than about 100, less than about 50 centiopoise. In some embodiments, the viscosity of the formulation is about 200, about 150, about 100, about 50 centiopoise. In particular embodiments, the viscosity is less than about 200 centiopoise. In others, less than about 120 centiopoise or less than about 100 centiopoise. In some embodiments, the viscosity is about 100 centiopoise. In others about 50 centiopoise. In still other embodiments the viscosity is about 200 centiopoise. Methods for measuring viscosity are well known to the skilled artisan. For example, as described in United States Pharmacopoeia 29 (Chapter 911) Viscosity, page 2785 (which is herein incorporated by reference in its entirety). As is well known to the skilled artisan, formulations commonly considered “gels” will have viscosity significantly greater than 1000 centiopoise, for example, greater than about 2000 centiopoise, greater than about 5000 centiopoise.

In some embodiments, including (but not limited to) where the use of salts is contraindicated as described above, the formulation may further include one or more tonicity agents.

As used herein, the term “tonicity agent” and its cognates refers to agents that adjust the tonicity of the formulation, but are not salts (e.g., not NaCl), which, as will be appreciated by the skill artisan in view of the teaching provided herein, are contraindicated for some formulations due to the presence of certain of the gel-forming polymers or viscosity-increasing agents. These agents may be used to prepare formulations that are suitable for the eye and are isotonic or near isotonic (e.g., somewhat hyper- or hypo-isotonic; e.g., within about ±20%, about ±15%, about ±10%, about ±5% of being isotonic). Tonicity agent(s) may also be used in formulations where the use of salts is not contraindicated.

Tonicity agents that may be used to adjust the tonicity of formulation the formulations described herein and that are suitable for administration to the eye are known to the skilled artisan and can be selected based on the teaching provided herein. For example, tonicity agents include polyols (e.g., sugar alcohols (e.g., mannitol, etc.), trihydroxy alcohols (e.g., glycerin, etc.), propylene glycol or polyethylene glycol, etc.), or combinations of two or more polyols. Likewise, the concentration of the tonicity agent(s) will depend upon the identity and concentrations of the other components in the formulation and can be readily determined by the skilled artisan in view of the teaching provided herein.

In certain embodiments, the tonicity agent is glycerin or mannitol. In some embodiments, the tonicity agent is glycerin. In others, mannitol. In still others a combination of mannitol and glycerin may be used.

Exemplary concentrations of tonicity agents include, for example from about 0.001 to about 3%. In some embodiments, the concentration of the tonicity agent (e.g., mannitol or glycerin) is, for example, about 0.001% to about 2.7%, about 0.001% to about 2.5%, about 0.001% to about 2%, about 0.001% to about 1.5%, about 0.001% to about 1%, about 0.01% to about 3%, about 0.01% to about 2.7%, about 0.01% to about 2.5%, about 0.01% to about 2%, about 0.01% to about 1.5%, about 0.01% to about 1%, about 0.1% to about 3%, about 0.1% to about 2.7%, about 0.1% to about 2.5%, about 0.1% to about 2%, about 0.1% to about 1.5%, about 0.1% to about 1%, about 0.01% about 1% to about 3%; about 1% to about 2.5%; about 1% to about 2%; about 1% to about 1.8%; about 1% to about 1.5%; or about 0.001%, about 0.01%, about 0.05%, about 0.08%, about 0.1%, about 0.2%, about 0.5%, about 0.8%, about 1%, about 1.5%, about 1.8%, about 2%, about 2.2%, about 2.5%, about 2.8%, or about 3% (w/v).

In certain embodiments, the tonicity agent is mannitol. In some of these embodiments, the carrier includes a gel-forming agent (e.g., gellan gum).

In some embodiments, the tonicity agent is mannitol. In certain of these embodiments, the carrier includes a viscosity-increasing agent (e.g., water soluble cellulose derivatives (e.g., hypromellose), polyvinyl alcohol, polyvinyl pyrrolidone, chondroitin sulfate, hyaluronic acid, or soluble starches).

In some embodiments, the formulation may additionally include a preservative (e.g., benzalkonium chloride, benzethonium chloride, chlorhexidine, chlorobutanol, methylparaben, Phenylethyl alcohol, propylparaben, thimerosal, phenylmercuric nitrate, phenylmercuric borate, or phenylmercuric acetate, peroxides), or a combination of two or more of the foregoing preservatives. In certain embodiments, the preservative is benzalkonium chloride.

As will be appreciated by the skilled artisan, preservatives may be present in concentrations of from about 0.001% to about 0.7% (w/v). In particular embodiments, the preservative(s) may be present in a concentration of from about 0.001% to about 0.5% (w/v); from about 0.001% to about 0.05% (w/v), from about 0.001% to about 0.02% (w/v), from about 0.001% to about 0.015% (w/v), from about 0.001% to about 0.005% (w/v), from about 0.01% to about 0.02%, from about 0.002% to about 0.01%, from about 0.015% to about 0.05%, less than about <0.5%, from about 0.005% to about 0.01%, from about 0.001% to about 0.15%, from about 0.002% to about 0.004%, from about 0.001% to about 0.002%. In some embodiments the concentration of the preservative may be, for example, about 0.001%, about 0.005%, about 0.01%, about 0.02%, about 0.03%, about 0.05%, about 0.1%, about 0.2%, about 0.5%, or about 0.7% (w/v).

Typical concentrations (w/v) for various commonly used preservatives are listed in Table 1, below.

TABLE 1

| | Approximate |
|---|---|
| | Concentration |
| Preservative | Range (w/v) |
| Benzalkonium chloride | 0.01-0.02% |
| Benzethonium chloride | 0.01-0.02% |
| Chlorhexidine | 0.002-0.01% |
| Chlorobutanol | <0.5% |
| Methylparaben | 0.015-0.05% |
| Phenylethyl alcohol | <0.5% |
| Propylparaben | 0.005-0.01% |
| Thimerosal | 0.001-0.15% |
| Phenylmercuric nitrate | 0.002-0.004% |
| Phenylmercuric borate | 0.002-0.004 |
| Phenylmercuric acetate | 0.001-0.002 |

In certain embodiments, the formulation may additionally include a surfactant, or combinations of two or more surfactants.

In particular embodiments, the formulation is substantially free of surfactant.

As used herein, the term “substantially free” is intended to refer to levels of a particular component that are undetectable using routine detection methods and protocols known to the skilled artisan. For example, HPLC (including chiral HPLC, chiral HPLC/MS, LC/MS/MS etc.), thin layer chromatography, mass spectrometry, polarimetry measurements, Gas-chromatography-mass spectrometry, or others.

In particular embodiments, the formulation may further include a chelating agent (e.g., edetate disodium (EDTA) (e.g., edetate disodium (dihydrate), etc.) citrates, etc.). In some embodiments, a combination of chelating agents may be present. As will be appreciated by those of skill in the field, chelating agents can be used to hinder degradation of the formulation components and thereby increase the shelf life of ocular formulations. As will be appreciated by the skilled artisan, use of EDTA in combination with gellan gum formulation may be contraindicated as the EDTA can cause gel formation prior to administration of the gellan gum formulation.

Typical concentrations for chelating agents are from about 0.005% to 0.1% (w/v). For example, from about 0.005% to about 0.09%, from about 0.005% to about 0.08%, from about 0.005% to about 07%, from about 0.005%, to about 0.06%, from about 0.005% to about 0.05%, from about 0.005 to about 0.04%, from about 0.005% to about 0.03%, from about 0.01% to about 0.1%, from about 0.01% to about 0.09%, from about 0.01% to about 0.08%, from about 0.01% to about 0.07%, from about 0.01% to about 0.06%, from about 0.01% to about 0.05%, from about 0.01% to about 0.04%, etc. In certain embodiments, the concentration of chelating agent(s) is about 0.005%, about 0.01%, about 0.02%, about 0.03%, about 0.05%, about 0.06%, about 0.07%, about 0.08%, about 0.09%, or about 0.1%.

In particular embodiments, the chelating agent is edetate disodium. In certain embodiments, the chelating agent is edetate disodium (dihydrate). In some of these embodiments, the edetate disodium dihydrate is present at a concentration of about 0.01% (w/v).

In some embodiments, the formulation may additionally include one or more buffering agents (e.g., phosphate buffer(s) (e.g., sodium phosphate buffers (e.g., dibasic sodium phosphate, monobasic sodium phosphate, etc.), citrate buffers, maleate buffers, borate buffers, etc.). As will be appreciated by the skilled artisan, the one or more buffering agent(s) should be selected in combination with the other components of a given formulation to achieve a pH suitable for use in the eye (e.g., pH of about 4.5 to about 8).

In certain embodiments, the buffering agent is a phosphate buffer or combination of two or more phosphate buffers. In certain embodiments, the buffering agents are dibasic sodium phosphate and monobasic sodium phosphate.

Typical concentrations for buffering agent(s) for example, phosphate buffering agent(s) may be from about 0.005 molar to 0.1 molar. In some embodiments, the buffering agent(s) may be at a concentration of about 0.01 to about 0.1, from about 0.01 to about 0.08, from about 0.01 to about 0.05, from about 0.01 to about 0.04, from about 0.02 to about 0.1, from about 0.02 to about 0.08, from about 0.02 to about 0.06, from about 0.02 to about 0.05, from about 0.02 to about 0.04 molar, etc. In particular embodiments, there are two buffering agents. Exemplary buffering agents include a combination of dibasic sodium phosphate (e.g., dibasic sodium phosphate.7H₂O) and monobasic sodium phosphate (e.g., monobasic sodium phosphate anhydrous). In some embodiments, the concentration of the buffering agent(s) is about 0.005 molar, about 0.01 molar, about 0.02 molar, about 0.03 molar, about 0.04 molar, about 0.05 molar, about 0.06 molar, about 0.07 molar, or about 0.1 molar.

An additional aspect of the invention includes use of the formulations as described herein in the manufacture of a medicament. Particularly, the manufacture of a medicament for use in the treatment and/or prevention of conditions as described herein. Further, the formulations, variously described herein, are also intended for use in the manufacture of a medicament for use in treatment and/or prevention of the conditions and, in accordance with the methods, described herein, unless otherwise noted.

Methods of Preparation

The pharmaceutical formulations described herein may be produced and evaluated as described in detail in the Examples, particularly Examples 1, 3, 4, and 6 and generally as described below and known to those of skill in the art. Additionally, the skilled artisan, based on the teachings provided herein and the particular formulation to be prepared will also be able to modify the preparation methods described herein and known in the art without undue experimentation.

Generally, formulations including mecamylamine, or a pharmaceutically acceptable salt thereof, and aqueous saline carrier can be routinely prepared by dissolving (e.g., sequentially (in any order) or simultaneously) sufficient quantities of mecamylamine (or a pharmaceutically acceptable salt thereof) and salt (e.g., NaCl, where present) in a sufficient volume of DI (deionized) water to achieve the desired concentration of mecamylamine and salt. Ranges for these components have been described in detail elsewhere in the present specification.

Dissolution may be aided by stirring, swirling, heating, etc, including combinations of two or more of the foregoing. Routine methods may be used to adjust the pH of the solution, if needed, to be suitable for topical administration to the eye. After the mecamylamine solution is prepared, it is generally advisable to filter the solution to remove any particulates prior to administration. The above protocol should be undertaken in sterile conditions and in accordance with GMP and GLP (Good Laboratory Practice) standards and, when intended for administration to humans, should also conform to regulatory guidelines, as will be appreciated by the skilled artisan.

Analysis, including confirmation of the concentration of mecamylamine present in the saline solution, may be performed by techniques known and available to the skilled artisan. For example, but not limited to, LC/MS/MS (e.g., as described in detail in Example 6), mass spectrometry (e.g., as described in detail in Example 6), etc. The skilled artisan will be able to further modify these techniques and other routine techniques based on the teaching provided herein and the information available in the field, thereby optimizing detection for the particular detection technique selected and the equipment utilized.

Additional components as described herein may be added (sequentially (in any order) or concurrently with mecamylamine (or pharmaceutically acceptable salts thereof) and salt (where present).

Where the formulation includes a gel-forming polymer as described herein, the formulation may be prepared as described in detail in Example 4, with the skilled artisan also able to modify the preparation according to methods known in the art, without undue experimentation in light of the teachings herein. The formulations including gel-forming polymer can also be analyzed as described above for the aqueous saline solutions of mecamylamine (or a pharmaceutically acceptable salt there), with particular reference to Example 6. Additionally, as described above for aqueous saline solutions of mecamylamine of other solutions of mecamylamine (e.g., polymer-free solution formulation with water as the carrier, solution with viscosity-increasing agent (e.g., hypromellose, etc.), etc.), the protocols for preparing the formulations should be undertaken in sterile conditions and in accordance with GMP and GLP standards and, when intended for administration to humans, should also conform to regulatory guidelines, as will be appreciated by the skilled artisan.

Generally, the pharmaceutical formulations of mecamylamine (or a pharmaceutically acceptable salt thereof) and a gel-forming polymer (where the final formulation is either a gel prior to topical ocular administration or forms a gel in situ upon topical ocular administration) can be produced as described in Example 5 and, more generally, by dissolving a particular amount of mecamylamine (or pharmaceutically acceptable salt thereof) in a given amount of water and then dispersing the gel-forming polymer in the mecamylamine-containing solution. The amounts of water, mecamylamine (or pharmaceutically acceptable salt thereof) and gel-forming polymer are dictated by the final concentration of the gel-forming polymer and mecamylamine for the particular formulation being prepared.

Generally, following the addition of the gel-forming polymer the solution will be mixed (e.g., by stirring, swirling, agitation, heating, or other routine methods, including combinations of two or more of the foregoing) for sufficient time to thoroughly disperse and dissolve the gel-forming polymer within the mecamylamine-containing solution. For example, the mixing may proceed for about 10 to about 60 minutes, about 15 to about 60 minutes, about 15 to about 45 minutes, about 15 to about 40 minutes, about 15 to about 30 minutes, about 15 to about 25 minute, at least about 10 minutes, at least about 15 minutes, at least about 20 minutes, at least about 30 minutes, at least about 40 minutes, at least about 60 minutes, about 10 minutes, about 15 minutes, about 20 minutes, about 30 minutes, about 40 minutes, about 50 minutes, about 60 minutes.

Thorough mixing of the components can be determined by visual inspection, light scattering, etc., as known to the skilled artisan.

Additional components, as described herein, may be added to the mecamylamine-containing solution prior to, concurrently with, or after the addition of the gel-forming polymer. Similarly, the additional components may alternatively be added to the water prior to, concurrently with or after the addition of mecamylamine (or a pharmaceutically acceptable salt thereof). In certain embodiments, the additional components are added to the water prior to or concurrently with addition of mecamylamine.

After the components have been thoroughly dissolved (including, optionally, any additional components as described herein), the resulting solution containing mecamylamine (or a pharmaceutical salt thereof), gel-forming polymer, water and, optionally, additional components can be equilibrated at room temperature. Not all formulations of mecamylamine will require equilibration.

The solution should be allowed to equilibrate for at least about 8 hours, at least about 10 hours, at least about 12 hours, at least about 16 hours, at least about 18 hours, at least about 24 hours, from about 8 to about 24 hours, from about 10 to about 24 hours, from about 10 to about 18 hours, from about 12 to about 18 hours, for about 8 hours, for about 10 hours, for about 12 hours, for about 14 hours, for about 16 hours, for about 18 hours, for about 20 hours, or for about 24 hours.

In certain embodiments, the solution is equilibrated for about 16 hrs. In other embodiments the solution is allowed to equilibrate for at least about 16 hours. In still other embodiments the solution is allowed to equilibrate for about 16 to about 24 hours.

The solutions and mixtures obtained as described herein may also be filtered to remove any particulates. Filtration should preferably be undertaken in sterile conditions. Routine methods known to those of skill in the art can be used to filter the solutions (e.g., under vacuum, by gravity, etc.) and appropriately-sized filters, based on the viscosity of the solution should be chosen (for example, but not limited to, use of 0.2 micron membrane filters is typical).

Particular methods for preparing topical ocular formulation that form a gel in situ upon administration to the eye are also described in, for example, U.S. Pat. Nos. 6,174,524 and 4,861,760, which are herein incorporated by reference in their entirety.

Use of the Formulations

Administration

As noted previously, in one aspect are provided methods of treating and/or prevention the conditions described herein using the pharmaceutical formulations as described herein. Unless clearly indicated otherwise by the context, the formulations described herein may be used without limitation in the methods herein described.

The methods may be practiced as a therapeutic approach towards the treatment and/or prevention of the conditions described herein. Thus, in certain embodiments, the pharmaceutical formulations may be used to treat and/or prevent the conditions described herein in individuals in need thereof, including humans. As described herein, the methods generally comprise topically administering to one or both eyes of an individual an amount of a formulation, as detailed herein, effective to treat and/or prevent the condition.

In particular embodiments, the methods include a) topically applying to one or both eyes of an individual in need thereof a formulation comprising mecamylamine, or a pharmaceutically acceptable salt thereof, and a carrier suitable for topical administration to the eye, wherein the mecamylamine or pharmaceutically acceptable salt thereof is present in the formulation in an amount sufficient to deliver a therapeutically effective amount of mecamylamine to one or more of the posterior tissues of the eye for the treatment or prevention of conditions mediated by retinal neovascularization, choroidal neovascularization, abnormal angiogenesis, vascular permeability, or combinations thereof, of posterior tissues of the eye. In some embodiments, the condition is a proliferative retinopathy or condition associated therewith.

In some embodiments, the methods include a) topically applying to one or both eyes of an individual in need thereof a formulation comprising mecamylamine, or a pharmaceutically acceptable salt thereof, and a carrier suitable for topical administration to the eye, wherein the mecamylamine or pharmaceutically acceptable salt thereof is present in the formulation in an amount sufficient to deliver a therapeutically effective amount of mecamylamine to one or more of the anterior tissues or fluids of the eye for the treatment or prevention of conditions mediated by neovascularization, abnormal angiogenesis, vascular permeability, or combinations thereof, of anterior tissues of the eye. In some embodiments, the condition involves abnormal angiogenesis affecting the anterior tissues of the eye or condition associated therewith.

In certain embodiments, when the formulation is topically administered to a rabbit eye, the ratio of the concentration of mecamylamine present in choroidal and retinal tissue, measured in units of ng/g, to the concentration of mecamylamine in plasma measured in units of ng/mL ([ng/g mecamylamine choroidal+retinal tissue]: [ng/mL plasma]) is at least about 40:1. In some embodiments, the ratio is at least about 20:1, at least about 25:1, at least about 30:1, or at least about 35:1.

In some embodiments, the ratio of the concentration of mecamylamine in retinal and choroidal tissue (ng/g):concentration of mecamylamine in plasma (ng/mL) when topically administered to a rabbit eye is at least about 20:1, at least about 25:1, at least about 30:1; at least about 35:1 at least about 45:1; at least about 50:1, at least about 60:1, at least about 70:1, at least about 80:1, at least about 100:1, at least about 150:1, at least about 200:1, at least about 300:1, at least about 350:1, at least about 375:1, at least about 400:1, at least about 425:1, at least about 450:1, at least about 475:1, at least about 500:1, at least about 550:1, at least about 600:1, at least about 650:1, at least about 700:1, at least about 750:1, at least about 800:1, at least about 850:1, at least about 900:1, at least about 950:1, at least about 1000:1, at least about 1025:1, at least about 1050:1, at least about 1100:1, at least about 1200:1, at least about 1300:1, at least about 1500:1, at least about 1700:1, at least about 2000:1 or at least 2500:1. In some embodiments, the ratio is from about 300:1 to about at 2500:1, from about 300:1 to about 2000:1, from about 300:1 to about 1500:1, from about 300:1 to about 1000:1, from about 300:1 to about 800:1, from about 350:1 to about at 2500:1, from about 350:1 to about 2000:1, from about 350:1 to about 1500:1, from about 350:1 to about 1000:1, from about 350:1 to about 800:1, from about 400:1 to about at 2500:1, from about 400:1 to about 2000:1, from about 400:1 to about 1500:1, from about 400:1 to about 1000:1, from about 400:1 to about 800:1, from about 450:1 to about at 2500:1, from about 450:1 to about 2000:1, from about 450:1 to about 1500:1, from about 450:1 to about 1000:1, from about 450:1 to about 800:1, from about 500:1 to about at 2500:1, from about 500:1 to about 2000:1, from about 500:1 to about 1500:1, from about 500:1 to about 1000:1, or from about 500:1 to about 800:1.

In particular embodiments, the ratio of the concentration of mecamylamine in retinal and choroidal tissue (ng/g):concentration of mecamylamine in plasma (ng/mL) when topically administered to a rabbit eye is at least about 20:1, at least about 25:1, at least about 30:1, at least about 35:1, at least about 40:1, at least about 50:1, at least about 80:1, at least about 100:1, at least about 300:1, at least about 40:1 to about 1000:1, from about 40:1 to about 1500:1, at least about 40:1 to about 2000:1, at least about 40:1 to about 2500:1, at least about 50:1 to about 250:1, at least about 80:1 to about 1000:1, at least about 80:1 to about 2000, at least about 100:1 to about 1000:1, at least about 100:1 to about 2000:1, at least about 200:1 to about 1000:1, or at least about 200:1 to about 2000:1. In particular embodiments, the ratio is at least about 300:1, at least about 350:1, at least about 450:1, at least about 500:1, at least about 1200:1, from about 300:1 to about 1000:1, from about 300:1 to about 2000:1, from about 350:1 to about 1000:1, from about 350:1 to about 2000:1, from about 450:1 to about 1000:1, from about 450:1 to about 1100:1, from about 450:1 to about 1200:1, from about 450 to about 2000:1, from about 500:1 to about 1000:1, from about 500:1 to about 1200:1, or about 500:1 to about 2000:1.

In some embodiments the ratio of the concentration of mecamylamine present in the corneal tissue when topically administered to a rabbit eye, measured in units of ng/g, to the concentration of mecamylamine in plasma measured in units of ng/mL ([ng/g mecamylamine corneal tissue]: [ng/mL plasma]) is at least about 1000:1. In some embodiments, the ratio of concentrations at least about 100:1, at least about 200:1, at least about 300:1, at least about 400:1, at least about 500:1 at least, at least about 600:1, at least about 700:1, about 800:1, at least about 850:1, at least about 900:1, at least about 950:1, at least about 1000:1, at least about 1025:1, at least about 1050:1, at least about 1100:1, at least about 1200:1, at least about 1300:1, at least about 1500:1, at least about 1700:1, at least about 2000:1 or at least 2500:1. In some embodiments, the ratio is from at least about 800:1 to about 4000:1, from at least about 800:1 to about 3000:1, from at least about 800:1 to about 2500:1, from at least about 900:1 to about 4000:1, from at least about 900:1 to about 3000:1, from at least about 1000:1 to about 4000:1, from at least about 1000:1 to about 3000:1, from at least about 1000:1 to about 2500:1, from at least about 1000:1 to about 2000:1. In certain embodiments, the ratio is at least about 850:1, at least about 900:1, at least about 1000:1 at least about 1200:1.

In some embodiments the ratio of the concentration of mecamylamine present in the aqueous humor when topically administered to a rabbit eye, measured in units of ng/mL, to the concentration of mecamylamine in plasma measured in units of ng/mL ([ng/mL mecamylamine aqueous humor]: [ng/mL plasma]) is at least about 40:1, at least about 45:1, at least about 50:1, at least about 55:1, at least about 60:1, at least about 70:1, at least about 80:1, at least about 100:1, at least about 150:1, at least about 200:1, or at least about 250:1. In some embodiments, the ratio is from about 40:1 to about at 2500:1, from about 40:1 to about 4000:1, at least about 40:1 to about 2500:1, from about 40:1 to about 1500:1, from about 40:1 to about 1000:1, from about 40:1 to about 800:1, from about 40:1 to about 500:1, about 40:1 to about 300:1, from about 40:1 to about 400:1, or from about 40:1 to about 100:1. In particular embodiments, the ratio is at least about 50:1.

In some embodiments, the individual is a mammal, including, but not limited to, bovine, horse, feline, rabbit, canine, rodent, or primate. In particular embodiments, the mammal is a primate. In certain embodiments, the primate is a human. In certain embodiments, the individual is human, including adults, children and premature infants. In some embodiments, the individual is not experiencing ocular growth. In some embodiments, the individual in an adult.

In certain embodiments the individual has been identified as having one or more of the conditions described herein. Identification of the conditions as described herein by a skilled physician is routine in the art and may also be suspected by the individual due to loss of vision or visual acuity (e.g., reduction in the field of vision, blurriness, etc.).

In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions (e.g., diagnosis or susceptibility to a “non-neovascular”/“dry” form of macular degeneration, etc.)), lifestyle or habits (for example, as previously described cigarette smoking is one of the leading risk factors for retinal neovascularization due to macular degeneration (e.g., age-related macular degeneration, etc.)). Certain patients are at risk of retinopathy. Individuals who are older, particularly those who are smokers, are more likely to have age-related macular degeneration, and are at risk of an associated proliferative retinopathy. Individuals with diabetes mellitus may develop a proliferative retinopathy. Premature infants are also at risk and are routinely screened for the development of retinopathy of prematurity. Individuals with non-neovascular forms of macular degeneration are particular at risk for development of the neovascular forms of macular degeneration.

The conditions that can be treated and/or prevented using the formulations and methods described herein include conditions that affect the posterior tissues of the eye, as well as conditions that affect the anterior tissues of the eye or the eye fluids. These conditions are described in greater detail below. In some cases, the conditions may affect one or more anterior tissues and more posterior tissues, for example, as in the case of an ocular tumor. Generally the conditions are mediated by neovascularization (also often referred to as angiogenesis) and, in particular abnormal angiogenesis.

The conditions amenable to treatment and/or prevention using the formulations and methods described herein include conditions mediated by retinal and/or choroidal neovascularization, abnormal angiogenesis, vascular permeability, or combinations thereof, of posterior tissues of the eye, including, but not limited to, proliferative retinopathies. In particular, proliferative retinopathies that are mediated by abnormal or increased angiogenesis and/or neovascularization of the posterior tissues of the eye, namely, the retina and choroid. Abnormal or increased angiogenesis and/or neovascularization is readily recognized by the skilled physician and can be identified and diagnosed using routine methods known in the art. (See for example, Ophthalmology. Clinical Signs and Differential Diagnosis, Jack J. Kanski and K. K. Nischall, Elsevier 1998, which is hereby incorporated by reference in its entirety.) For example, intravenous administration of fluoroscein and subsequent illumination with ultra violet light is one means of identifying the presence of angiogenesis and/or neovascularization, as the blood vessels resulting from the angiogenesis and/or neovascularization are characterized by a propensity to leak blood or fluid from the vessels. This leakage can be visualized using fluoroscein. New blood vessels resulting from abnormal or increased angiogenesis and/or neovascularization are also characterized by a greater degree of branching than typical blood vessels observed in healthy individuals (e.g., individuals not suffering from vision loss and/or impairment of visual acuity), tend to be smaller in diameter than blood vessels usually found in the particular tissue type and also tend to appear in, or appear in greater number/density than expected for the particular tissue type or location.

Proliferative retinopathies mediated by angiogenesis include, but are not limited to retinal neovascularization due to macular degeneration (e.g., wet forms (e.g., neovascular forms), age-related maculopathy, age-related macular degeneration (“AMD”) (e.g., wet forms), diabetic retinopathy, retinopathy of prematurity (also commonly referred to as retrolental fibroplasia), retinopathy due to sickle cell disease, etc. (See for example, Ophthalmology: Clinical Signs and Differential Diagnosis, Jack J. Kanski and K. K. Nischall, Elsevier 1998, which is hereby incorporated by reference in its entirety.) The “dry” or “non-neovascular” forms of macular degeneration are often an early indication that an individual is susceptible to, or may develop, a “neovascular” or “wet form” of macular degeneration. (See Bressler et al., (1990) Arch. Ophthalmol. 108(10):1442-7 “Relationship of drusen and abnormalities of the retinal pigment epithelium to the prognosis of neovascular macular degeneration. The Macular Photocoagulation Study Group.”) Thus treatment to prevent neovascularization may be warranted for these individuals (e.g., preventative or prophylactic treatment). It is possible that such early treatment, even where prior to the identification of neovascularization, may prevent the occurrence of detectable macular degeneration due to neovascularization. Thus, it is intended that the “dry” or “non-neovascular” form of macular degeneration is also a condition intended to be treated using the methods and formulations herein described.

In particular embodiments, the condition is diabetic retinopathy, retinopathy of prematurity, retinal neovascularization due to macular degeneration, or retinopathy due to sickle cell disease. In certain embodiments, the condition is diabetic retinopathy. In other embodiments, the condition is retinopathy of prematurity. In still other embodiments, the condition is retinal neovascularization due to macular degeneration. In certain embodiments, the condition is retinopathy due to sickle cell disease.

In particular embodiments, where the condition is retinal neovascularization due to macular degeneration, the condition may be age-related macular degeneration (AMD). In certain embodiments, the AMD may be the “wet” form of AMD (e.g., neovascular form). In other embodiments, the AMD may be the “dry” form of AMD (e.g., non-neovascular form). Characterization of the various forms of AMD is well studied and known to the skilled artisan.

In some embodiments, the condition to be treated and/or prevented may be associated with macular edema.

Conditions associated with neovascularization, abnormal angiogenesis, vascular permeability, (or combinations thereof) of the anterior tissues of the eye include corneal neovascularization, pterygium, post-transplant neovascularization, rubeosis iridis, neovascular glaucoma, ocular tumors, etc. In some embodiments, the condition is corneal neovascularization. In other cases, the condition is pterygium. In particular embodiments the conditions is rubeosis iridis.

In some embodiments, the anterior tissue of the eye affected by neovascularization, abnormal angiogenesis, vascular permeability, or a combination thereof, is the cornea, lens, iris, sclera, or trabecular meshwork. In particular embodiments, the affected tissue is the cornea. In others, the lens. In particular embodiments, the lens, cornea or iris is affected.

The terms, “pharmaceutically effective amount” or “therapeutically effective amount,” and cognates of these terms, as used herein refer to an amount of a formulation sufficient to treat a specified condition (e.g., disease, disorder, etc.) or one or more of its symptoms and/or to prevent the occurrence of the condition. In reference to ocular conditions mediated by neovascularization and/or abnormal angiogenesis (e.g., proliferative retinopathies, etc), a pharmaceutically or therapeutically effective amount comprises an amount sufficient to, among other things, cause a reduction in the presence of newly formed blood vessels or a decrease the rate angiogenesis and/or neovascularization, and/or to reduce the leakage of fluid and/or bleeding from these vessels. In certain embodiments, the pharmaceutically effective amount is sufficient to prevent the condition, as in being administered to an individual prophylactically. For example, administration of the formulations described herein to individuals with non-neovascular macular degeneration would prevent the occurrence of the neovascular form of macular degeneration. As another example, the formulation could be administered to an individual who has developed a pterygium that is not yet interfering with vision, so as to prevent the further growth of the pterygium, as a prophylactic measure to prevent its interfering with vision.

With respect to the conditions affecting the posterior tissues of the eye described herein, for a treatment to be therapeutically effective, a sufficient amount of mecamylamine should be delivered to the posterior region of the eye. For example, the retina and/or choroid, which are the tissues in the posterior region of the eye in which the conditions described herein are first manifested. Later, such vessels may extend into the vitreous compartment. In certain embodiments, a therapeutically effective amount of mecamylamine is delivered to the retina and the choroid. In some embodiments, a therapeutically effective amount of mecamylamine is delivered to the retina. In particular embodiments, a therapeutically effective amount of mecamylamine is delivered to the choroid. Where the sclera is affected, the effective amount of mecamylamine should be delivered to the appropriate portion of the sclera (e.g., either anterior portion, posterior portion or both). In some embodiments, a therapeutically effective amount of mecamylamine is delivered to the sclera. In some embodiments, a therapeutically effective amount of mecamylamine is delivered to the posterior portion of the sclera. In some embodiments, a therapeutically effective amount of mecamylamine is delivered to both the anterior and posterior portion of the sclera.

With respect to the conditions affecting the anterior tissues of the eye described herein, for a treatment to be therapeutically effective, a sufficient amount of mecamylamine should be delivered to the anterior region of the eye. For example, the cornea, lens, trabecular meshwork, or iris, which are the tissues in the anterior region of the eye in which the conditions described herein are manifested. Where the sclera is affected, the effective amount of mecamylamine should be delivered to the appropriate portion of the sclera (e.g., either anterior portion, posterior portion or both). In certain embodiments, a therapeutically effective amount of mecamylamine is delivered to the cornea and the lens. In some embodiments, a therapeutically effective amount of mecamylamine is delivered to the cornea. In particular embodiments, a therapeutically effective amount of mecamylamine is delivered to the lens. In some embodiments, a therapeutically effective amount of mecamylamine is delivered to the sclera. In some embodiments, a therapeutically effective amount of mecamylamine is delivered to the anterior portion of the sclera. In some embodiments, a therapeutically effective amount of mecamylamine is delivered to both the anterior and posterior portion of the sclera.

The formulations and methods described herein may be used alone or in conjunction with (e.g., prior to, concurrently with, or after) other modes of treatments (e.g., adjunctive therapy with additional agents used to treat or prevent the condition being treated and/or administration of an additional treatment modality, or combinations thereof). For example, in combination with one or more additional (non-mecamylamine) pharmaceutical agents (also referred to as therapeutic agents) as described herein and known to those of skill in the art and/or currently available treatment modalities, including thermal laser photocoagulation or photodynamic therapy. As used herein, the term “additional treatment modality” refers to treatment of the conditions described herein without the use of a pharmaceutical agent (e.g., thermal laser photocoagulation, photodynamic therapy, etc.). Where combinations of pharmaceutical agent(s) and/or additional treatment modality(ies) are used, they may be, independently, administered prior to, concurrently with, or after administration of the topical ocular formulation of mecamylamine.

As will be well appreciated by the skilled artisan, for particular conditions, different pharmaceutical agent(s) and/or additional treatment modality(ies) may be indicated. For example, in conjunction with the treatment described herein, treatment of rubeosis iridis may also include treatment of the associated glaucoma (e.g., use of pharmaceutical agents (e.g., dichlorphenamide, carbochol, demacarium bromide, etc.). When the condition is neovascular glaucoma (often associated with diabetic retinopathy), an additional pharmaceutical agent may be agents to lower intra-ocular pressure (e.g., steroids); additional treatment modalities may include laser photocoagulation. Where pterygium is the condition, one or more pharmaceutical agent(s) (e.g., artificial tears, anti-inflammatory agents, etc.) may be warranted, and, possible, additional treatment modalities such as e.g., laser or surgical ablation.

In some embodiments, the pharmaceutical agent(s) may be an nAChR antagonist, anti-inflammatory agent (e.g., NSAID, etc.), VEGF antagonist, VEGF scavenger (e.g., VEGF TRAP, etc.), tyrosine kinase inhibitor, prostaglandin receptor antagonist, agent used in the treatment of glaucoma, or an agent to lower intra-ocular pressure, as described previously. Combinations of two or more of the foregoing may also be administered, as can be determined by the skilled artisan in view of the teaching provided herein.

In certain embodiments, the pharmaceutical agent(s) may be, for example, not limited to, one or more nAChR antagonists (e.g., such as those described above with regard to the formulations of mecamylamine). In some embodiments, the pharmaceutical agent(s) may be an anti-inflammatory agent (e.g., NSAID). In certain embodiments, the pharmaceutical agent(s) may be an tyrosine kinase inhibitor. In certain embodiments, the pharmaceutical agent(s) may be an prostaglandin receptor antagonist. In some embodiments, the pharmaceutical agent(s) may be an agent used in the treatment of glaucoma. In some embodiments, the pharmaceutical agent(s) may be an agent to lower intra-ocular pressure.

In some embodiments, the pharmaceutical agent(s) may include one or more pharmaceutical agents shown to be effective in the treatment of the conditions described herein. For example, VEGF antagonists (e.g., anti-VEGF (vascular endothelial growth factor) antibodies or fragments thereof, VEGF apatamers (e.g., pegaptanib sodium). In certain embodiments, the anti-VEGF antibodies are monoclonal antibodies. Exemplary anti-VEGF antibodies include, but are not limited to, bevacizumab and ranibizumab (tradenames AVASTIN® and LUCENTIS®, respectively, under development by Genentech, Inc., South San Francisco, Calif.). Pharmaceutical agents may also include the Vascular Endothelial Growth Factor (VEGF) receptor antagonist pegaptanib (an aptamer) (MACUGEN®; Pfizer). In some embodiments, the pharmaceutical agent is a VEGF scavenger (e.g., VEGF TRAP, etc.). In some variations, the pharmaceutical agent is a VEGF scavenger, VEGF antagonist, or tyrosine kinase inhibitor.

The formulations described herein can be administered in conjunction with one or more of the pharmaceutical agents as described herein and as known in the art, one or more additional agents to further reduce the occurrence and/or severity of side effects (including adverse reactions) and/or clinical manifestations thereof (for example, agents which inhibit mydriasis), or in conjunction with (e.g., prior to, concurrently with, or after) thermal laser photocoagulation or photodynamic therapy. However, as noted previously, based on current clinical data and non-human in vivo animal testing it appears that the side effects are limited in occurrence and severity and thus many individuals will not need the administration of additional pharmaceutical reagents to reduce and/or prevent these effects. The formulations as described herein may be administered before, concurrently with, or after the administration of one or more of the pharmaceutical agents described herein. The formulations thereof described herein may also be administered in conjunction with (e.g., prior to, concurrently with, or after) agents to alleviate the symptoms associated with either the condition or the treatment regimen. For example, in certain variations, thermal laser photocoagulation or photodynamic therapy may be administered to the individual prior to administration of mecamylamine. In some variations thermal laser photocoagulation or photodynamic therapy may be administered to the individual after administration of mecamylamine. In particular variations, thermal laser photocoagulation or photodynamic therapy may be administered to the individual throughout the course of treatment with mecamylamine.

Where pharmaceutical agents are administered in conjunction with the mecamylamine formulations described herein, the additional agents may be administered parenterally or orally. For example, intravenously, via injection, orally, topically, via biodegradable implants, etc. Given the difficulty of identifying and formulating drugs for topical ocular delivery, many, if not most, pharmaceutical agents will not be formulated for topical ocular delivery, but will instead be administered in accordance with established protocols for the particular agent.

The optimal combination of one or more of surgery and/or other additional treatment modalities and/or additional pharmaceutical agents in conjunction with administration of the formulations described herein, can be determined by an attending physician based on the individual and taking into consideration the various factors effecting the particular individual, including those described herein.

Formulation and Dosage

As noted previously, the pharmaceutical formulations as described herein may be topically administered to one or both eyes of individuals in need thereof for the treatment or prevention of conditions as described herein in conjunction with the methods of use described herein.

The formulations described herein will generally be used in an amount effective to achieve the intended result, for example in an amount effective to treat or prevent the particular condition being treated. The formulations may be administered therapeutically to achieve therapeutic benefit. By therapeutic benefit is meant eradication or amelioration of the underlying condition being treated and/or eradication or amelioration of one or more of the symptoms associated with the underlying condition such that the patient reports an improvement in feeling or condition, notwithstanding that the patient may still be afflicted with the underlying condition. Therapeutic benefit also includes halting or slowing the progression of the condition, regardless of whether improvement is realized.

In some embodiments, where the condition being treated is a proliferative retinopathy, an effective amount is an amount sufficient to reduce the rate of angiogenesis and/or neovascularization of the retina and/or choroid (e.g., as measured by visual acuity (e.g., as with a Snellen chart), retinal edema (e.g., as with Optical Coherence Tomography) or vascular permeability (e.g., as with fluorescein angiography) prior to and/or after treatment). In certain embodiments, an effective amount is an amount sufficient to decrease existing neovascularization (e.g., where results of one or more of the clinical tests listed above after treatment is reduced compared to the same clinical test (or combination of tests) prior to treatment). As will be appreciated by the skilled artisan, similar, and additional, diagnostic methods can be used to monitor treatment progress for conditions affecting the anterior tissues of the eye.

The amount of the formulations administered in order to administer an effective amount of mecamylamine, or a pharmaceutically acceptable salt thereof, will depend upon a variety of factors, including, for example, the particular condition being treated, the frequency of administration, the particular formulation being administered, the severity of the condition being treated and the age, weight and general health of the individual, the adverse effects experienced by the individual being treated, etc. Determination of an effective dosage is within the capabilities of those skilled in the art in view of the teachings provided herein.

In certain embodiments, the unit dose of mecamylamine administered at a particular time will be determined by the assessment of visual acuity (e.g., as measured by clinical tests (e.g., as with a Snellen chart), retinal edema (e.g., as with Optical Coherence Tomography) or vascular permeability (e.g., as with fluorescein angiography) prior to and/or after treatment). In certain embodiments, an effective amount is an amount sufficient to decrease existing neovascularization (e.g., where the results of one or more of the above-listed clinical tests performed after treatment is reduced compared to the results of the same one or more clinical tests performed prior to treatment).

In certain embodiments, the unit dose of mecamylamine administered at a particular time will be from about 0.01 mg/eye to about 15 mg/eye. For example, from about 0.01 mg/eye to about 7.5 mg/eye. In some embodiments, the dose administered will be from about 0.01 mg/eye to about 10 mg/eye, from about 0.01 mg/eye to about 5 mg/eye, from about 0.01 mg/eye to about 3 mg/eye, from about 0.01 mg/eye to about 1 mg/eye, from about 0.01 mg/eye to about 2 mg/eye, 0.03 mg/eye to about 10 mg/eye, from about 0.05 mg/eye to about 5 mg/eye, from about 0.05 mg/eye to about 3 mg/eye, from about 0.05 mg/eye to about 1 mg/eye, from about 0.05 mg/eye to about 2 mg/eye, 0.1 mg/eye to about 10 mg/eye, from about 0.5 mg/eye to about 5 mg/eye, from about 0.5 mg/eye to about 3 mg/eye, from about 0.5 mg/eye to about 2 mg/eye, from about 0.5 mg/eye to about 1 mg/eye; from about 1 mg/eye to about 10 mg/eye, from about 1 mg/eye to about 7 mg/eye, from about 1 mg/eye to about 5 mg/eye, from about 1 mg/eye to about 3 mg/eye, or from about 1 mg/eye to about 2 mg/eye; about 0.1 mg/eye, about 0.3 mg/eye, about 0.5 mg/eye, about 0.7 mg/eye, about 0.9 mg/eye, about 1 mg/eye, about 1.2 mg/eye, about 1.5 mg/eye, about 1.7 mg/eye, about 2 mg/eye, about 2.2 mg/eye, about 2.5 mg/eye, about 2.7 mg/eye, about 3 mg/eye, about 3.2 mg/eye, about 3.5 mg/eye, about 3.7 mg/eye, about 4 mg/eye, about 4.5 mg/eye, about 5 mg/eye, about 5.5 mg/eye, about 6 mg/eye, about 6.5 mg/eye, about 7 mg/eye, about 7.5 mg/eye, about 8 mg/eye, about 8.5 mg/eye, about 9 mg/eye, about 9.5 mg/eye, about 10 mg/eye, about 10.5 mg/eye, about 11 mg/eye, about 12 mg/eye, about 13 mg/eye, about 14 mg/eye or about 15 mg/eye. In certain embodiments, the dose at a particular administration is 0.05 mg/eye to about 1 mg/eye.

In particular embodiments, the total daily dose is from about 0.01 mg/eye to about 7.5 mg/eye per day. For example, twice daily administration of doses of from about 0.005 mg/eye to about 3.75 mg/eye. In others, For example, twice daily administration of doses of from about 0.05 mg/eye to about 0.5 mg/eye. In some embodiments, the total daily dose is from about 0.1 mg/eye to about 3 mg/eye per day. In other embodiments, the total daily dose is from about 0.1 mg/eye to about 0.7 mg/eye, from about 0.1 mg/eye to about 0.5 mg/eye, or from about 0.1 mg/eye to about 0.3 mg/eye. In certain embodiments, the total daily dose is from about 0.1 mg/eye to about 1 mg/eye.

As will be appreciated by the skilled artisan, the dose administered at a given time and the selection of the concentration of mecamylamine, or pharmaceutically acceptable salt thereof, should also take into account the volume of formulation that can be accommodated by an individual's eye. For example, the dosing schedule may need to be altered to when mecamylamine is administered to premature infants for the treatment retinopathy of prematurity, as, in addition to a lower dosage being indicated due to body weight and, likely general health, the infant eye will also accommodate a lower volume of the formulation. However, such alterations and adjustments should be well within the skill of the attending physician without undue experimentation in light of the teachings provided herein.

In some embodiments, the volume of formulation administered per eye may be from about 50 μl to about 1 mL. In certain embodiments, the volume of formulation administered per eye may be from about 10 μl to about 500 μL. In certain embodiments, the volume of formulation administered per eye may be from about 10 μl to about 1 mL. For example, from about 100 μl to about 400 μL, from about 10 μl to about 300 μL, from about 10 μl to about 200 μL, from about 10 μl to about 100 μL, from about 10 μl to about 50 μL, from about 30 μl to about 500 μL, from about 30 μl to about 400 μL, from about 30 μl to about 300 μL, from about 30 μl to about 200 μL, from about 30 μl to about 100 μL, from about 30 μl to about 50 μL, from about 50 μl to about 100 μl, from about 50 μl to about 90 μl, from about 500 μl to about 80 μl, from about 50 μl to about 70 μl, from about 50 μl to about 60 μl, from about 60 μl to about 100 μl, from about 70 μl to about 100 μl, from about 80 μl to about 100 μl, from about 90 μl to about 100 μl, about 110 μl, about 100 μl, about 90 μl, about 80 μl, about 70 μl, about 60 μl, about 50 μl, 90 μl to about 100 μl, from about 90 μl to about 200 μl, from about 90 μl to about 300 μl, from about 90 μl to about 400 μl, from about 90 μl to about 500 μl, from about 90 μl to about 600 μl, from about 90 μl to about 700 μl, from about 90 μl to about 800 μl, from about 90 μl to about 900 μl, about 1 mL, about 900 μl, about 800 μl, about 700 μl, about 600 μl, about 500 μl, about 400 μl, about 450 μl, about 350 μl, about 300 μl, about 250 μl, about 200 μl, about 100 μl, about 90 μl, about 80 μl, about 70 μl, about 60 μl, or about 50 μl.

The dose administered may be higher or lower than the dose ranges described herein, depending upon, among other factors, the particular formulation used, the tolerance of the individual to adverse side effects, the frequency of administration, and various factors discussed above. Dosage amount and interval may be adjusted individually to provide retinal/choroidal tissue levels of the mecamylamine that are sufficient to maintain therapeutic effect, according to the judgment of the prescribing physician. Skilled artisans will be able to optimize effective local dosages without undue experimentation in view of the teaching provided herein.

Dosages may also be estimated using in vivo animal models.

Multiple doses of the formulations as described herein may also be administered to individuals in need thereof over the course of hours, days, weeks, or months. For example, but not limited to, daily, twice per day, three times per day, four times per day, every other day, once weekly, twice weekly, etc. In certain embodiments, the formulations are administered daily, twice per day or three times per day. In particular embodiments, the formulations are administered twice a day or once a day. In some embodiments, the formulations are administered once a day. In others, twice a day.

Kits

Also provided are kits for topical ocular administration of the formulations described herein.

In certain embodiments the kits may include a dosage amount of at least one pharmaceutical formulation as disclosed herein. Kits may further comprise suitable packaging and/or instructions for use of the formulation. Kits may also comprise a means for the delivery of the pharmaceutical formulation thereof, such as an eye dropper for the administration of solutions and in situ gel-forming solutions as described herein, or other device as described herein and known to those of skill in the art, particular to aid in the administration of the formulations when the formulation is in the form of gel prior to administration.

The kits may include other pharmaceutical agents for use in conjunction with the mecamylamine formulations described herein. In certain embodiments, the pharmaceutical agent(s) may be one or more other nAChR antagonist(s). These agents may be provided in a separate form, or mixed with the compounds of the present invention, provided such mixing does not reduce the effectiveness of either the pharmaceutical agent or formulations described herein and is compatible with topical administration to the eye. Similarly the kits may include additional agents for adjunctive therapy. For example, agents to reduce the adverse effects of the mecamylamine or other agents known to the skilled artisan as effective in the treatment of the conditions described herein.

The kits will include appropriate instructions for preparation and administration of the formulation, side effects of the formulation, and any other relevant information. The instructions may be in any suitable format, including, but not limited to, printed matter, videotape, computer readable disk, or optical disc.

In another aspect of the invention, kits for treating an individual who suffers from or is susceptible to the conditions described herein are provided, comprising a first container comprising a dosage amount of a formulation as disclosed herein, and instructions for use. The container may be any of those known in the art and appropriate for storage and delivery of intravenous formulations. In certain embodiments the kit further comprises a second container comprising a pharmaceutically acceptable carrier, diluent, adjuvant, etc. for preparation of the composition to be administered to the individual.

Kits may also be provided that contain sufficient dosages of the formulations as disclosed herein to provide effective treatment for an individual for an extended period, such as 1-3 days, 1-5 days, a week, 2 weeks, 3, weeks, 4 weeks, 6 weeks, 8 weeks, 3 months, 4 months, 5 months, 6 months, 7 months, 8 months, 9 months or more.

Kits may also include multiple doses of the formulations and instructions for use and packaged in quantities sufficient for storage and use in pharmacies, for example, hospital pharmacies and compounding pharmacies.

The kits may include the formulations as described herein packaged in either a unit dosage form or in a multi-use form. The kits may also include multiple units of the unit dose form.

In certain embodiments, are provided the formulations described herein in a unit dose form. In other embodiments the formulations may be provided in a multi-dose form (e.g., a container of solution for dispensing drops of the solution, etc.).

All patents, patent applications and publications referred to herein are hereby incorporated herein by reference in their entirety.

### EXAMPLES

The present invention is further described with reference to the following Examples; however, these Examples are not limiting the scope of the present invention.

Materials

Unless otherwise noted, the chemicals and other reagents used throughout the Examples were obtained from commercial suppliers as reagent grade and used without further purification.

Mecamylamine hydrochloride USP (Poli Industria Chimica, Milan, Italy), ketamine, xylazine, GELRITE®, NaCl, NaOH, HCl, DMSO (dimethylsulfoxide), sodium EDTA (ethyelenediaminetretraacetate), acetonitrile, formic acid, dextromethorphan, diphenhydramine, methanol. Water used was deionized water (DI).

### Example 1

### Parenteral Formulation of Mecamylamine

TABLE 2
Parenteral Formulation (IV)

| Ingredient | % (wt./vol) |
|---|---|
| Mecamylamine hydrochloride | 30 mg/mL |
| Sterile Sodium Chloride | Qs to make isotonic (0.9% NaCl) |
| solution for injection | |

Parenteral formulations of mecamylamine hydrochloride were prepared by dissolving Ig mecamylamine hydrochloride USP (white powder) and 33.33 mL 0.9% sterile NaCl in approximately in a volumetric flask. The mixture was manually stirred at room temperature until mecamylamine powder was completely dissolved resulting in a clear solution. The pH of the solution was adjusted to 7.4 using NaOH and HCl.

### Example 2

### Ocular Bioavailability Following Intravenous Administration

This study was designed to model the ocular bioavailability of mecamylamine when administered systemically. Rabbit eyes are the preferred model for in vivo modeling of ocular drugs, however, the rabbit is not the subject of choice for modeling oral bioavailability. However, systemic administration does emulate orally administered mecamylamine to a reasonable approximation since mecamylamine has rapid absorption and high oral bioavailability. Therefore, intravenous injection was used to model ocular bioavailability of mecamylamine administered systemically, in order to determine the deposition of mecamylamine to the plasma, vitreous and posterior tissues (retina/choroid) of the eye from the blood.

The study comprised 2 groups (each N=6, 12 rabbits total) of male NZW (New Zealand White) rabbits weighing approximately 2.5-3 kg and obtained from Kralek Farms (Turlock, Calif.). Mecamylamine solution, prepared as described in Example 1, at a dose of 15 mg/kg was delivered via i.v. infusion as either a short infusion (1 hr) or slow infusion (6 hr) in rabbits sedated by ketamine/xylazine, with the slow infusion intended to model controlled systemic release of the mecamylamine.

For both groups, samples of the vitreous(≧0.1 ml) and plasma were withdrawn at 6 time points: pre-dose, 30 min, 1, 2, 4, and 6 hr. At 6 hours, all animals were sacrificed and vitreous and retinal tissues were collected. The total amount of mecamylamine administered during each infusion was the same, based on the weight of the individual rabbit.

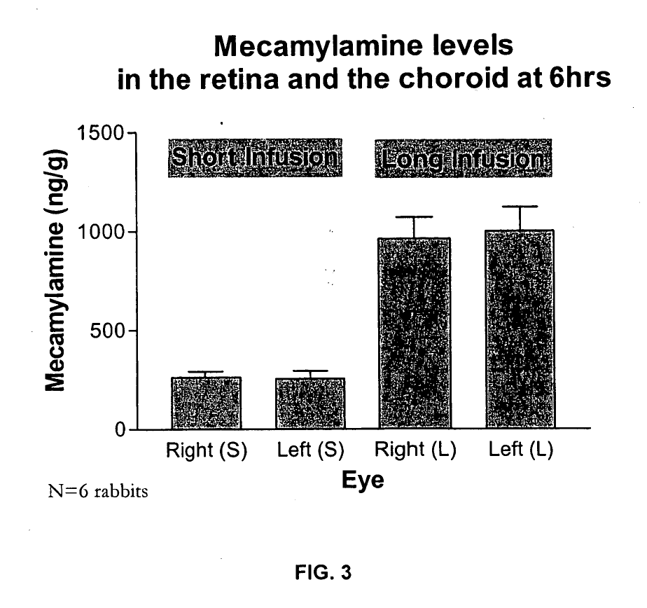

The concentrations of mecamylamine present in the various samples were analyzed as described in Example 6, below. The concentration of mecamylamine in plasma (ng/mL) and vitreous (ng/mL) for the short infusion are shown in FIG. 1. The concentration of mecamylamine in plasma (ng/mL) and vitreous (ng/mL) for the long infusion are shown in FIG. 2. The concentration amount of mecamylamine in the retinal/choroidal tissues following the long infusion are shown in FIG. 3, while FIG. 4A shows a comparison of the plasma, vitreal, and retina/choroid levels of mecamylamine for the long infusion.

As is apparent from FIGS. 1 and 2, systemic administration of the same amount of mecamylamine over either period leads to a greater Cₘₐₓ, for mecamylamine in the plasma compared to the vitreous, though for the long infusion the last time point shows an increasing concentration in the vitreous relative to the plasma concentration. FIG. 3 shows that mecamylamine, administered systemically, preferentially deposits in the retina/choroid when administered over a long infusion, with the amount of mecamylamine reaching the retina/choroid being more than 2-fold greater for the long infusion than for the short infusion.

### Example 3

### Preparation of Topical Ophthalmic Solution Formulation

TABLE 3
Isotonic Ophthalmic Formulation

| Ingredient | % (wt./vol.) | |
|---|---|---|
| Mecamylamine HCl | 2.0 | g |
| NaCl | 0.9 | g |
| DI Water | To 100 | mL |

Mecamylamine hydrochloride USP was dissolved 100 mL of DI water. A 0.9 g weight of sodium chloride was then added with stirring to make the solution isotonic (0.9% NaCl w/v). The solution was then filtered through a 0.2 micron membrane filter and packaged under sterile conditions.

### Example 4

### Preparation of Topical In Situ Gel-forming Ophthalmic Formulation

TABLE 4
In Situ Gel-forming Ophthalmic Formulation

| Ingredient | % (wt./vol.) | |
|---|---|---|
| Mecamylamine HCl | 2.0 | g |
| GELRITE ® | 0.6 | g |
| DI Water | 100 | mL |

Mecamylamine HCl USP (2.0 g) was dissolved with stirring in 100 mL DI water. The GELRITE® powder (0.6 g) was then dispersed by shaking in the aqueous solution of mecamylamine. The dispersion was then stirred for 20 minutes using a mechanical shaker (Vortex). After 20 minutes of stirring the GELRITE® solution dissolved and a solution was formed. The solution was then equilibrated at room temperature for approximately 16 h. The solution was then packaged under sterile conditions.

Unless otherwise noted, the gel-forming polymer content (GELRITE®) was 0.6% (w/v).

### Example 5

### Ocular Bioavailability Following Topical Ocular Administration

The objective of this study was to determine the pharmacokinetics of mecamylamine, administered either as an isotonic solution (prepared as in Example 3) or in situ gel-forming solution (as prepared in Example 4), in the plasma, vitreous humor and retina of the eyes when applied topically to the surface of the eye.

The study comprised 2 groups (N=4/group; 8 rabbits total) of male NZW rabbits each weighing approximately 2-3 kg and obtained from Kralek Farms (Turlock, Calif.). Mecamylamine hydrochloride was formulated as described in Examples 3 and 4 and 100 μL was administered to each eye as either an in situ gelling solution (group 1) or isotonic solution (group 2) to the cornea of each eye and the bottom eyelid is separated from the surface of the eye to make a pocket to ensure the entire dose was retained in the eye (see Table 5 for Study design). For both groups, samples of the vitreous(≧0.1 ml) were withdrawn at 6 time points (one withdrawal per eye): pre-dose, 30 min, 1 (sacrifice), 3, 6, 12, and 24 (sacrifice) hr. At all time points, only duplicate vitreous fluid samples (each coming from different eye from different animals) were collected in order to minimize pain and damage to rabbit eyes.

Blood for plasma(≧0.5 ml) and intra-vitreal fluid (˜0.1 ml) were collected at 0, 30, 60 min from each animal with sacrifice for 2 animals/group at 60 minutes. At 3, 6, and 12 hours, blood for plasma, (˜2 ml) from each remaining animal, and intravitreal fluid, (˜100 μl) (duplicate sample only at each time point) from 2 additional animals, were collected. All remaining animals were sacrificed at 24 hours with both blood and vitreous fluid collected. The blood was collected into microtainer tubes using sodium EDTA as anti-coagulant.

At either 60 minutes or 24 hours, animals were sacrificed and the vitreous and retina (including choroid) were collected. The blood was also collected and centrifuged to separate the plasma. The plasma is separated from the red cell pellet and both were frozen at −80° C. individually for each time point.

Plasma, red cell pellet, vitreous (entire vitreous collected upon rabbit sacrifice, but split into two aliquots) and retina, including choroid, samples were frozen in liquid N₂. The plasma and the retina (including choroid) were stored at −80° C. and, when shipped for analysis, were packaged with dry ice to prevent decomposition of the samples.

During the vitreous fluid tapping and blood collection periods, rabbits were lightly sedated with a Ketamine/Xylazine mixture to minimize discomfort and to facilitate the procedures. The plasma and fluid were immediately placed into labeled Eppendorf® tubes and frozen at −80° C. Samples were maintained at −80° C. until packaged with dry ice and shipped for analysis.

TABLE 5
Study Design

| | Animal | Taps | Sacri- | Animal | | Sacri- |
|---|---|---|---|---|---|---|
| Group | No. | (min) | fice | No. | Taps (hr) | fice |
| 1 | 101, 102 | 0, 30 | 60 min | 103, 104 | 3, 6, 12 | 24 h |
| 2 | 201, 202 | 0, 30 | 60 min | 203, 204 | 3, 6, 12 | 24 h |

### Example 6

### Sample Preparation

Plasma and Vitreous Sample Preparation:

A 0.5 mg/mL stock of mecamylamine hydrochloride USP in DMSO was prepared and used as the working standard to produce the calibration standards for quantification of the mecamylamine content in various samples collected as described above. Calibration standards were prepared by diluting the 0.5 mg/mL standard 1 in 100 into plasma to 5 μg/mL (5 μL+495 μL), then diluting further with plasma by 3-fold serial to obtain a mecamylamine concentration of 2.29 ng/mL.

Calibration standards, quality control (QC) samples, and plasma, erythrocytes and vitreous study samples were prepared for HPLC injection by precipitating 50 μL of plasma with 3× volumes (150 μL) of ice-cold acetonitrile containing 100 ng/mL of dextromethorphan and 50 ng/mL of diphenhydramine as internal standards. Following centrifugation at 6000 g for 30 min, 40 μL of each supernatant was diluted with 200 μL of 0.2% formic acid in water.

Retinal/Choroid Sample Preparation

Each tissue sample collected was weighed. Following weighing, 1 μL of water was added per mg of tissue followed by 3× volumes (relative to water) of ice-cold Internal Standard Solution (acetonitrile containing 100 ng/mL of dextromethorphan and 50 ng/mL of diphenhydramine). Samples were homogenized using an electric rotor/stator-type homogenizer (Tissue Tearor). Following homogenization, a 200 μL aliquot of each homogenate was then centrifuged and diluted as described above. The samples were analyzed using LC/MS/MS and quantified using the calibration standards prepared in plasma as described above.

LC/MS/MS conditions:

HPLC: Shimadzu VP System

Mobile Phase: 0.2% formic acid in water (A) and 0.18% formic acid in methanol (B)

Column: 2×10 mm Higgins Phalanx C18 guard cartridge

Injection Volume: 100 μl

Gradient: 5-95% B in 2 minutes after a 0.5 minute wash

Flow Rate: 400 μl/min

Mass Spectrometer: Applied Biosystems/MDS SCIEX API 3000 (Applied Biosystems Inc., Fremont, Calif.)

Interface: TurbolonSpray (ESI) at 400° C.

Polarity: Positive Ion

Q1/Q3 Ions: 168.2/137.2 for Mecamylamine

256.2/167.2 for Diphenhydramine (Internal standard)

272.1/215.2 for Dextromethorphan (Internal standard)

Dosing Solution Analysis.

The mecamylamine dosing solutions (labeled “A” and “B”) and the 0.5 mg/mL working standard (described above) were diluted 1 in 100 into DMSO by volume. These solutions were analyzed by LC/MS/MS using the conditions listed above and the concentration of mecamylamine in the dose solutions was calculated relative to the mecamylamine reference standard in DMSO.

Results:

The calibration standards, QC samples, and plasma, erythrocytes and vitreous study samples were prepared for HPLC injection and analyzed on Day 1. The retina study samples were prepared for HPLC injection and analyzed, along with the calibration standards and QC samples, on Day 5. The dosing solutions were analyzed concurrent with the retina samples.

Diphenhydramine was used as the internal standard and each calibration curve was fit using power regression. Samples with a concentration value above 5000 ng/mL were re-analyzed by injecting 1/10 the original injection volume to get the analyte peak on scale.

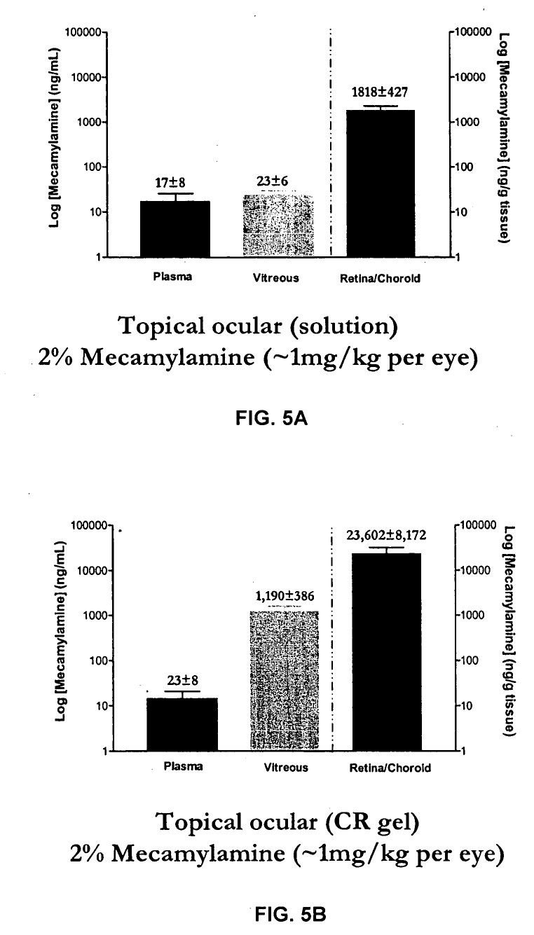

The data from the topical administration studies presented in Example 5 and analyzed as described in Example 6 are presented in FIGS. 4A, 5A-B, and 6A-B.

From a comparison of FIGS. 4A, 4B and 5A it is apparent that administration of mecamylamine HCl via systemic administration results in greater relative amounts of mecamylamine appearing in plasma compared to the amount appearing in the retina/choroid, when compared to the relative amounts appearing when mecamylamine is administered topically (either as a solution or as a in situ gel-forming solution). Quite unexpectedly the ratio of mecamylamine (ng/g) present in retinal/choroidal tissue to the mecamylamine concentration in plasma (ng/mL) is at least about 40-fold greater for topical administration compared to systemic administration. Thus, for a given dose of mecamylamine administered topically, far less mecamylamine will appear in the plasma compared to the retina/choroid and thus a therapeutic dose can be achieved without the side effects often experienced during the administration of mecamylamine. The data also show that the levels of mecamylamine present in the erythrocytes (red blood cells) and the plasma are comparable, indicating that the mecamylamine in the systemic circulation has not been sequestered in the red blood cells. Thus the concentration of systemic levels of mecamylamine (as measured by either plasma or red blood cells) relative to ocular tissue levels is surprisingly low and indicates that the adverse effects associated with systemic administration of mecamylamine (e.g., CNS effects, etc.) should not be experienced by individuals when administered the topical ocular formulations of mecamylamine locally via the eye.

Additionally, formulation of the mecamylamine as a topical in situ gel-forming solution has an even greater effect on favorably partitioning the mecamylamine in the retinal/choroidal tissue, with a ratio of concentration of mecamylamine in retinal/choroidal tissue (ng/g) to the mecamylamine concentration in plasma (ng/mL) being at least about 450:1 for the in situ gel-forming solution. The relative amounts for the various formulations and routes of administration are tabulated below in Table 6.

TABLE 6

| | Ratio of concentration |
|---|---|
| | of mecamylamine in |
| | retinal/choroidal tissue |
| | (ng/g):concentration of |
| Formulation/Delivery Route | mecamylamine in plasma (ng/mL) |
| Long i.v. Infusion (systemic) | ˜1.4:1 to ˜2.1:1 |
| Isotonic Ophthalmology | ˜81.8:1 to ˜204:1 |
| Solution (topical) | |
| In situ Gel-forming | ˜497:1 to ˜39900:1 |
| Solution (topical) | |

### Example 7

### Single- and Multiple-dose Rabbit Ocular Pharmacokinetics

Dutch-belted male rabbits (Covance, Denver, Pa.) aged 2.5 to 4.5 months and with weights of 1.5 to 2.5 kg were given a 3% solution of mecamylamine hydrochloride (polymer-free solution) by ocular installation either as a single dose or 6 doses (50 microliters per dose), approximately 1.5 hours apart. Serial blood samples (approximately 0.5 mL each) were collected by direct venipuncture from a marginal ear vein into a blood collection tube containing EDTA as anticoagulant from various animal groups at 0.5 hours post dose, 2 hours post dose. A terminal blood sample was obtained from all animals just prior to euthanasia. Animals were euthanized 1, 3 or 6 hours after administration of the drug solution. Ocular tissues (listed in Table 7) were obtained after euthanasia.

The concentration of mecamylamine in plasma and ocular tissues was measured using an LC MS/MS method as described in Example 6. Results are presented in Table 7.

TABLE 7
Concentration of Mecamylamine in Tissues of Rabbits Following
Ocular Instillation of 3% Solution of Mecamylamine HCl

| | Time | Single dose | | | Multiple dose(6×/day) | | |
|---|---|---|---|---|---|---|---|
| Tissue | (hr) | N | Mean | STD | N | Mean | STD |
| Aqueous | 1 | 6 | 2492 | 568 | 6 | 3423 | 1080 |
| humor | 3 | 8 | 820 | 437 | ND | ND | ND |
| | 6 | 8 | 311 | 200 | ND | ND | ND |
| Con- | 1 | 6 | 7052 | 4799 | 6 | 10005 | 5299 |
| junctiva | 3 | 8 | 5420 | 3035 | ND | ND | ND |
| | 6 | 8 | 4164 | 2632 | ND | ND | ND |
| Cornea | 1 | 6 | 24817 | 8213 | 6 | 56517 | 38054 |
| | 3 | 8 | 14569 | 6336 | ND | ND | ND |
| | 6 | 8 | 25655 | 22541 | ND | ND | ND |
| Extraocular | 1 | 6 | 8260 | 6340 | 6 | 798 | 535 |
| muscle | 3 | 8 | 1780 | 3040 | ND | ND | ND |
| | 6 | 8 | 400 | 706 | ND | ND | ND |
| Iris/Ciliary | 1 | 6 | 9952 | 6179 | 6 | 84383 | 22634 |
| Body | 3 | 8 | 12338 | 7523 | ND | ND | ND |
| | 6 | 8 | 18011 | 8918 | ND | ND | ND |
| Lens | 1 | 6 | 111 | 88 | 6 | 227 | 136 |
| | 3 | 8 | 28 | 28 | ND | ND | ND |
| | 6 | 8 | 31 | 32 | ND | ND | ND |
| Optic | 1 | 6 | 706 | 703 | 6 | 504 | 519 |
| Nerve | 3 | 8 | 441 | 360 | ND | ND | ND |
| | 6 | 8 | 178 | 129 | ND | ND | ND |
| Retina/ | 1 | 6 | 510 | 280 | 6 | 2572 | 1933 |
| choroid | 3 | 8 | 171 | 101 | ND | ND | ND |
| | 6 | 8 | 420 | 279 | ND | ND | ND |
| Sclera/ | 1 | 6 | 4738 | 2009 | 6 | 13372 | 2782 |
| Anterior | 3 | 8 | 5303 | 2529 | ND | ND | ND |
| | 6 | 8 | 3230 | 1219 | ND | ND | ND |
| Sclera/ | 1 | 6 | 4457 | 2626 | 6 | 3378 | 2019 |
| Posterior | 3 | 8 | 2853 | 3075 | ND | ND | ND |
| | 6 | 8 | 949 | 1019 | ND | ND | ND |
| Vitreous | 1 | 6 | 251 | 300 | 6 | 202 | 163 |
| | 3 | 8 | 60 | 66 | ND | ND | ND |
| | 6 | 8 | 27 | 20 | ND | ND | ND |
| Plasma | 1 | 3 | 15 | 6 | 3 | 43 | 16 |
| | 3 | 4 | 11 | 15 | ND | ND | ND |
| | 6 | 4 | 5 | 8 | ND | ND | ND |

Multiple dosing was 6 times per day at hourly intervals.
ND = Not done.

Mecamylamine was found in high concentrations from anterior to posterior of the rabbit eyes. Mean peak levels in aqueous humor were approximately 310 to 920 ng/mL, and in the retina/choroid were 171 to 510 ng/g in tissue 1 hour to 6 hours after dosing. Concentrations remained high through the six hours of sampling. Relatively little mecamylamine was seen in the vitreous. Plasma levels were low—on the order of 50 ng/mL or less. When examined 1 hour after 6 hourly doses, a mecamylamine concentration in the retina/choroid was five-fold that of single dose. There was some accumulation seen in aqueous humor and blood, but none in the vitreous humor. The ratio of mecamylamine concentration in the retina/choroid to the plasma was high (37-147×).

Topical ocular administration of the mecamylamine formulation was well tolerated in the rabbits and no adverse clinical signs were observed following administration.

### Example 8

### Single Ocular Dose Drug Distribution in Rabbits

A single dose of a polymer-free solution, gellan gum formulation and hypromellose formulation containing 3% mecamylamine hydrochloride, was instilled in the eyes of rabbits (Dutch-belted male rabbits (Convance, Denver, Pa.) minimum age 2 months and with weights of 1.6 to 1.8 kg). Two animals were euthanized 30 minutes, 1 hour or 3 hours after dosing.

Sample of blood and ocular tissue (obtained as described in previous examples) were analyzed for mecamylamine concentration using an LC MS/MS method up to 3 hours after dosing. Resulting are summarized in the Table 8.

TABLE 8
Concentration of mecamylamine (ng/mL or ng/g) in the
tissues/fluids of rabbits following a single ocular dose
of 3% mecamylamine HCl in polymer-free solution

| Tissue | Time (hr) | N | Mean | STD |
|---|---|---|---|---|
| Aqueous humor | 0.5 | 4 | 8678 | 1223 |
| | 1 | 4 | 3045 | 706 |
| | 3 | 4 | 820 | 295 |
| Conjunctiva | 0.5 | 4 | 18650 | 9089 |
| | 1 | 4 | 11498 | 7702 |
| | 3 | 4 | 40575 | 11568 |
| Cornea | 0.5 | 4 | 88425 | 24955 |
| | 1 | 4 | 36000 | 14745 |
| | 3 | 4 | 24675 | 10701 |
| Extraocular muscle | 0.5 | 4 | 16825 | 7298 |
| | 1 | 4 | 11433 | 8516 |
| | 3 | 4 | 3699 | 4377 |
| Iris/Ciliary body | 0.5 | 4 | 67250 | 41732 |
| | 1 | 4 | 78200 | 34631 |
| | 3 | 4 | 37700 | 17072 |
| Lens | 0.5 | 4 | 288 | 96 |
| | 1 | 4 | 229 | 51 |
| | 3 | 4 | 307 | 97 |
| Optic nerve | 0.5 | 4 | 3357 | 3549 |
| | 1 | 4 | 3138 | 2178 |
| | 3 | 4 | 5085 | 7419 |
| Retina/choroid | 0.5 | 4 | 4955 | 4224 |
| | 1 | 4 | 6207 | 6960 |
| | 3 | 4 | 915 | 519 |
| Anterior sclera | 0.5 | 4 | 20360 | 13590 |
| | 1 | 4 | 13210 | 2955 |
| | 3 | 4 | 5958 | 3096 |
| Posterior sclera | 0.5 | 4 | 8193 | 7761 |
| | 1 | 4 | 5495 | 4178 |
| | 3 | 4 | 2888 | 2384 |
| Vitreous | 0.5 | 4 | 231 | 211 |
| | 1 | 4 | 116 | 49 |
| | 3 | 4 | 46 | 12 |
| Plasma | 0.5 | 6 | 35 | 23 |
| | 1 | 2 | 18 | 3 |
| | 2 | 2 | 37 | 44 |
| | 3 | 2 | 0 | 0 |

Mecamylamine administered as the polymer-free solution was found in high concentrations moving anteriorly to posteriorly in the eye. Mean peak levels in aqueous humor were approximately 7700 to 9100 ng/mL, and in the retina/choroid were 5000 to 11300 ng/mL measured between 30 minutes to 3 hours after dosing. Concentrations remained high through the three hours of sampling. Relatively little mecamylamine was seen in the lens and the vitreous. Plasma levels were low—on the order of 50 ng/mL or less.

The area-under-the-curve (AUC) was calculated using a trapezoidal rule on a population pharmacokinetic basis (see Table 9). The bioavailability of mecamylamine into the retina/choroid was ˜7500 ng/g·hr when administered as the polymer-free solution.

Thus, it appears that mecamylamine delivered as a topical ocular formulation penetrated the eyes, and reached the posterior pole of the eye—perhaps through a scleral route. The systemic bioavailability from the ocular route was low, and the ratio of intraocular to systemic levels was high. No safety issues arose during the course of the study.

TABLE 9
Mecamylamine AUC in Rabbit Tissues following a Single
Ocular Instillation of 3% solutions (polymer-free,
gellan gum and hypromellose) of mecamylamine HCl
(ng/mL · hour or ng/g · hour)

| | AUC | AUC | AUC |
|---|---|---|---|
| Matrix | (polymer free) | (Gellan Gum) | (HPMC) |
| Aqueous humor | 7501 | 8690 | 7355 |
| Conjunctiva | 96224 | 130013 | 114398 |
| Cornea | 111563 | 11725 | 113938 |
| Extraocular muscle | 21527 | 45501 | 31236 |
| Iris-ciliary body | 148125 | 159388 | 114813 |
| Lens | 872 | 1841 | 1064 |
| Optic Nerve | 13418 | 19018 | 11515 |
| Retina/Choroid | 7410 | 15145 | 17804 |
| Anterior sclera | 28700 | 38756 | 32790 |
| Posterior sclera | 12620 | 29363 | 23638 |
| Vitreous humor | 266 | 552 | 335 |

Calculated using trapezoidal rule on population values

### Example 9

### Distribution of Mecamylamine into Ocular Tissue of Rabbits Following Intravenous Administration

Mecamylamine was administered intravenously to male New Zealand white rabbits (approximately 2.5-3 kg (Kralek Frams, Turlock, Calif.)) at a dose of 15 mg/kg (dissolved in sterile 0.9% NaCl for injection) over either over 60 minutes or 6 hours. Drug concentrations were measured as described previously at different time points in the plasma, the vitreous and at 6 hours in the retina-choroid tissue. For both groups, the vitreous(>0.1 mL) was withdrawn at 8 time points, with each animal being sampled no more than twice in total: pre-dose, 5 minutes, 15 minutes, 30 minutes, 1, 2, 4, and 6 hours. At all time points, only two vitreous samples were collected in order to minimize pain and damage to rabbit eyes, in compliance with IACUC guidelines. Plasma samples were also collected at the same 8 time points: pre-dose, 5 minutes, 15 minutes, 30 minutes, 1, 2, 4, and 6 hours, with duplicate samples collected at each time point. At 6 hours, the animals were sacrificed and the vitreous and retina-choroid tissue were collected from all animals. Clinical observations were recorded periodically throughout the study. Biological samples for this study were analyzed using an LC/MS/MS method (see Example 6) with a lower limit of quantitation of 0.5 ng/mL.

Results of analysis of drug concentration in the plasma are shown below in Table 10.

TABLE 10
Concentration of mecamylamine in the plasma of rabbits
following intravenous infusion of mecamylamine (ng/mL)

| | Group | | | | | |
|---|---|---|---|---|---|---|
| | MEC15 mg/kg i.v. 1 hr | | | MEC15 mg/kg i.v. 6 hr | | |
| Hour | N | Mean | Std | N | Mean | Std |
| 0 | 6 | 48.7 | 45 | 6 | 0.0 | 0 |
| 0.5 | 6 | 5330.7 | 8816 | 6 | 350.5 | 77 |
| 1 | 6 | 1818.3 | 603 | 6 | 470.0 | 63 |
| 2 | 6 | 454.7 | 148 | 6 | 571.5 | 103 |
| 4 | 6 | 227.3 | 101 | 6 | 717.0 | 91 |
| 6 | 6 | 102.5 | 54 | 6 | 313.0 | 115 |

As expected, plasma levels showed a higher peak mean concentration with the infusion of the drug over a shorter period compared to the longer period (5,331 ng/mL vs. 717 ng/mL). Peak levels were seen within 0.5 hours with the shorter period, whereas they were relatively constant over the longer infusion period.

Results of analysis of drug concentration in the vitreous humor are shown below in Table 11.

TABLE 11
Concentration of mecamylamine in the vitreous humor of rabbits
following intravenous infusion of mecamylamine (ng/mL)

| | Group | | | | | |
|---|---|---|---|---|---|---|
| | MEC15 mg/kg i.v. 1 hr | | | MEC15mg/kg i.v. 6 hr | | |
| Hour | N | Mean | Std | N | Mean | Std |
| 0 | 2 | 43.0 | 11.7 | 2 | 5.6 | 5.8 |
| 0.5 | 2 | 716.6 | 881.7 | 2 | 59.9 | 14.8 |
| 1 | 2 | 1109.5 | 212.8 | 2 | 138.1 | 66.4 |
| 2 | 2 | 899.5 | 33.2 | 2 | 223.0 | 84.9 |
| 4 | 2 | 429.5 | 7.8 | 2 | 408.0 | 145.7 |
| 6 | 12 | 173.8 | 48.9 | 12 | 457.5 | 115.7 |

Note:
The differential sample size per group reflects the tissue sampling.

Mecamylamine levels in the vitreous paralleled the time course seen in the plasma with levels in the vitreous, ranging up to a mean of 1110 ng/mL with the shorter infusion period (seen at 1 hour) and a mean of 458 ng/mL with the longer infusion period (seen at 6 hours).

Results of analysis of drug concentration in the retina/choroid are shown below in Table 12.

TABLE 12
Concentration of mecamylamine in the retina/choroid of rabbits
following intravenous infusion of mecamylamine (ng/mL)

| | Group | | | | | |
|---|---|---|---|---|---|---|
| | MEC15 mg/kg i.v. 1 hr | | | MEC15 mg/kg i.v. 6 hr | | |
| Hour | N | Mean | Std | N | Mean | Std |
| 6 | 12 | 260.8 | 80.0 | 12 | 1534.3 | 1926.2 |

Source: Report: CB05-5160-O-PK (2005)

In the retina/choroid, sampled only at sacrifice at 6 hours, mean levels were 261 ng/mL for the shorter period, and 1,534 ng/mL for the longer period.

### Example 10

### 6-Hour Evaluation of the Ocular Pharmacokinetics of a 2% Mecamylamine GELRITE Solution Following Topical Instillation

The objective of this study was to evaluate the ocular pharmacokinetics of a 2% Mecamylamine in a GELRITE solution up to 6 hours following topical instillation into the eyes of New Zealand White rabbits. Nine New Zealand White female rabbits of minimum age of 9 weeks and weight 2-3 kg were obtained from The Rabbit Source (Ramona, Calif.) were used in the study. The dosing regime is shown in Table 13.

TABLE 13
6-Hour Evaluation of the Ocular Pharmacokinetics of a 2% Mecamylamine Solution
Following Topical Instillation in New Zealand White Rabbit Eyes: Dosing regimen

| | | Ocular Treatment | Ocular Treatment | | | | |
|---|---|---|---|---|---|---|---|
| | | (Left Eye, | (Right Eye, | | Pupil Diameter and Pupillary | Necropsy | |
| | | Topical | Topical | Dose | Response Observations | (Time Post- | |
| Group | No. | Instillation) | Instillation) | Volume | (Time Post-Dose) | Dose) | |
| A | 3 | 2% | Vehicle Control | 2 × 50 μL | −15, 15, 30, and 45 minutes; 1 | 1 | hour |
| | | Mecamylamine | | | hour | | |
| B | 2 | 2% | 2% | 2 × 50 μL | −15, 15, 30, and 45 minutes; 1 | 1 | hour |
| | | Mecamylamine | Mecamylamine | | hour | | |
| C | 2 | 2% | 2% | 2 × 50 μL | −15, 15, 30, and 45 minutes; | 3 | hours |
| | | Mecamylamine | Mecamylamine | | 1, 1.5, 2, 2.5, and 3 hours | | |
| D | 2 | 2% | 2% | 2 × 50 μL | −15, 15, 30, and 45 minutes; | 6 | hours |
| | | Mecamylamine | Mecamylamine | | 1, 1.5, 2, 2.5, 3, and 6 hours | | |

On day 1, 2 drops of 50 μL of mecamylamine or vehicle were administered topically into the appropriate eye(s) of each animal as described in Table 13. The time of each dose administration was recorded. There was no mortality in the treatment group.

Pupil size. No apparent differences in pupil diameter (horizontal or vertical) were observed in 2% mecamylamine-treated eyes over the course of the study. A mild decrease in pupil diameter was seen at the 15 minute timepoint when compared to the 15 minute (pre-dosing) timepoint in 2% Mecamylamine-treated eyes, but this decrease was not substantial or consistent. Pupillary response was normal for all eyes at all observation timepoints.

With respect to ocular distribution of mecamylamine, mean values for ocular tissues are shown in Table 14. In the choroid, the concentration of mecamylamine one hour after dosing unilaterally was ˜2800 ng/g. The fellow untreated eye in this group of animals had a mean concentration of mecamylamine of ˜700 ng/g (suggesting that there is delivery of the drug to the fellow eye). One hour after dosing bilaterally, the concentration of mecamylamine was ˜14,000 ng/g, decreasing three hours later to ˜500 ng/g, and six hours later to ˜260 ng/g. The levels of mecamylamine in the retina were similar to that seen in the choroid. Mecamylamine was also seen in relatively high concentrations in the cornea, aqueous humor, but not in the vitreous humor. Mean levels in the plasma and packed cells were approximately 5 to 38 ng/mL, highest one hour after dosing with bilateral dosing. Levels of mecamylamine in the plasma and packed cells were similar, thus there was no evidence of sequestration in the red blood cells (Table 15).

TABLE 14
Mecamylamine concentration in ocular tissues (ng/g)

| | Group | | | | | | | | | | | | | | |
|---|---|---|---|---|---|---|---|---|---|---|---|---|---|---|---|
| Matrix/ | MEC OS 1 hr | | | MEC OU 1 hr | | | MEC OU 3 hr | | | MEC OU 6 hr | | | MEC fellow | | |
| Hour | N | Mean | Std | N | Mean | Std | N | Mean | Std | N | Mean | Std | N | Mean | Std |
| Aqueous | | | | | | | | | | | | | | | |
| humor | | | | | | | | | | | | | | | |
| 1 | 3 | 2513.3 | 705.7 | 4 | 3907.5 | 2855.1 | ND | ND | ND | ND | ND | ND | 3 | 9.7 | 2.1 |
| 3 | ND | ND | ND | ND | ND | ND | 4 | 499.5 | 94.4 | ND | ND | ND | ND | ND | ND |
| 6 | ND | ND | ND | ND | ND | ND | ND | ND | ND | 4 | 48.9 | 26.8 | ND | ND | ND |
| Choroid | | | | | | | | | | | | | | | |
| 1 | 3 | 2806.7 | 176.2 | 4 | 14170.0 | 12230.1 | ND | ND | ND | ND | ND | ND | 3 | 705.7 | 248.2 |
| 3 | ND | ND | ND | ND | ND | ND | 4 | 1086.0 | 513.0 | ND | ND | ND | ND | ND | ND |
| 6 | ND | ND | ND | ND | ND | ND | ND | ND | ND | 4 | 264.5 | 156.0 | ND | ND | ND |
| Cornea | | | | | | | | | | | | | | | |
| 1 | 3 | 9600.0 | 2656.1 | 4 | 17892.5 | 10586.9 | ND | ND | ND | ND | ND | ND | 3 | 652.3 | 335.0 |
| 3 | ND | ND | ND | ND | ND | ND | 4 | 3825.0 | 454.6 | ND | ND | ND | ND | ND | ND |
| 6 | ND | ND | ND | ND | ND | ND | ND | ND | ND | 4 | 1232.5 | 672.8 | ND | ND | ND |
| Retina | | | | | | | | | | | | | | | |
| 1 | 3 | 4160.0 | 1103.2 | 4 | 14710.0 | 12889.4 | ND | ND | ND | ND | ND | ND | 3 | 781.3 | 373.1 |
| 3 | ND | ND | ND | ND | ND | ND | 4 | 563.0 | 272.8 | ND | ND | ND | ND | ND | ND |
| 6 | ND | ND | ND | ND | ND | ND | ND | ND | ND | 4 | 309.8 | 158.5 | ND | ND | ND |
| Vitreous | | | | | | | | | | | | | | | |
| humor | | | | | | | | | | | | | | | |
| 1 | 3 | 37.8 | 6.4 | 4 | 130.6 | 80.9 | ND | ND | ND | ND | ND | ND | 3 | 10.4 | 1.0 |
| 3 | ND | ND | ND | ND | ND | ND | 4 | 17.7 | 11.6 | ND | ND | ND | ND | ND | ND |
| 6 | ND | ND | ND | ND | ND | ND | ND | ND | ND | 4 | 5.9 | 1.5 | ND | ND | ND |

TABLE 15
Mecamylamine concentration in blood (ng/mL)

| | Group | | | | | | | | | | | |
|---|---|---|---|---|---|---|---|---|---|---|---|---|
| | MEC OS 1 hr | | | MEC OU 1 hr | | | MEC OU 3 hr | | | MEC OU 6 hr | | |
| Matrix/Hour | N | Mean | Std | N | Mean | Std | N | Mean | Std | N | Mean | Std |
| Cell | | | | | | | | | | | | |
| Fraction | | | | | | | | | | | | |
| Pre | 3 | 0.0 | 0 | 2 | 0.0 | 0 | 2 | 0.0 | 0 | 3 | 0.0 | 0 |
| Post | 9 | 15.0 | 1 | 6 | 33.1 | 26 | 6 | 7.0 | 6 | 9 | 1.2 | 0 |
| Plasma | | | | | | | | | | | | |
| Pre | 3 | 0.0 | 0 | 2 | 0.0 | 0 | 2 | 0.0 | 0 | 1 | 0.0 | |
| Post | 9 | 16.3 | 2 | 6 | 37.8 | 31 | 6 | 8.3 | 7 | 3 | 0.3 | 0 |

### Example 11

### Safety of Topical Ocular Instillation of a Saline Solution Containing Mecamylamine HCl

A phase 1 study entitled: “A Phase 1, Double-Masked, Randomized, 1-Day and 14-Day Escalating Dose Study to Assess the Ocular and Systemic Safety of Mecamylamine Ophthalmic Solution” was initiated in the United States in healthy adults. The safety objectives of this study were to evaluate the ocular and systemic safety of 4 concentrations of mecamylamine ophthalmic solution following 1-day (administered twice) and 14-day (BID) administration. The dose levels of mecamylamine ophthalmic solution evaluated were 0.03%, 0.1%, 0.3%, and 1%. A summary of the composition of the mecamylamine ophthalmic solution and placebo formulation is shown below in Table 7.

In the 1-day portion of the study, 10 subjects in each group were dosed in a single eye on two occasions with 6 hours between dosing. Within each group 8 subjects received mecamylamine ophthalmic solution and 2 subjects received vehicle (placebo) only. In the 14-day portion, 10 subjects were dosed twice daily at 12-hourly intervals in both eyes for 14 consecutive days. Subjects were evaluated for local (ocular) safety and tolerability based upon evaluation of ocular symptoms, ocular comfort, best-corrected visual acuity, biomicroscopy with fluorescein staining, measurement of intraocular pressure (IOP) by Goldman applanation tonometry, ophthalmoscopy, pupil size, and Schirmer's Test. They were also evaluated for systemic safety based upon assessment of physical examination, 12-lead ECG, vital signs, adverse events, hematology, clinical chemistry, and, urinalysis.

As of Oct. 26, 2006, 70 of the total 80 planned subjects were treated. Dosing is currently ongoing for the final dosing group of 10 subjects (ATG003 1% BID×14 days). The results to date have indicated no treatment-related indications of ocular or systemic toxicity. Specifically:
All subjects in all dosing groups rated the comfort level as being either “Very Comfortable” or “Comfortable” following study drug administration. No subject in any of the dosing groups experienced “Uncomfortable” or “Intolerable” symptoms at any time.
There have not been any significant changes in best-corrected visual acuity in any subject.
No clinically relevant treatment emergent effects noted in any subject based on biomicroscophic (slit lamp) examination with dilation and fluorescein staining. In particular, there have not been any observations of corneal erosions or ulcers, anterior chamber abnormalities, conjunctival irritation or redness, or any lens or retinal abnormalities noted in any subject.
No abnormal or clinically relevant treatment-emergent increases in intraocular pressure were noted in any subject.
No change in pupil size noted in any subject.
No treatment-emergent changes in tear production noted in any subject.
No clinically relevant changes have been noted in pulse or blood pressure (including postural changes) in any subject.
There have been no serious or severe adverse events reported in any subject. All adverse events reported to date have been of mild severity, and of a transient nature. No subject has discontinued study medication as a result of an adverse event. Significantly, there has not been any evidence of a drug-related increase in adverse effects resulting from systemic ganglionic blockade (such as constipation, urinary retention, postural hypotension, or dry mouth).
No clinically relevant changes have been noted in ECG parameters in any subject.

No clinically relevant changes have been noted any laboratory value (hematology, clinical chemistry, and, urinalysis):

TABLE 16
Composition of Mecamylamine Ophthalmic Solution and Placebo:

| | | | Test Articles by Strength of Active (w/v) | | | |
|---|---|---|---|---|---|---|
| Ingredient | Function | Placebo | 0.03% | 0.10% | 0.30% | 1.00% |
| Mecamylamine | Active | NA | 0.03 | 0.10 | 0.30 | 1.00 |
| hydrochloride | Pharmaceutical | | | | | |
| | Ingredient | | | | | |
| Monobasic | Buffer | 0.097 | 0.097 | 0.097 | 0.097 | 0.097 |
| sodium | | | | | | |
| phosphate | | | | | | |
| monohydrate | | | | | | |
| Dibasic sodium | Buffer | 0.322 | 0.322 | 0.322 | 0.322 | 0.322 |
| phosphate | | | | | | |
| heptahydrate | | | | | | |
| Sodium Chloride | Tonicity | 0.80 | 0.80 | 0.77 | 0.71 | 0.50 |
| | Adjustment | | | | | |
| Benzalkonium | Preservative | 0.01 | 0.01 | 0.01 | 0.01 | 0.01 |
| Chloride | | | | | | |
| Edetate Disodium | Metal chelator | 0.01 | 0.01 | 0.01 | 0.01 | 0.01 |
| Dihydrate | | | | | | |
| HCl Solution/ | pH Adjustment | 7.2 | 7.2 | 7.2 | 7.2 | 7.2 |
| Sodium | | | | | | |
| Hydroxide | | | | | | |
| solution (adjust | | | | | | |
| to pH 7.2) | | | | | | |
| Purified Water | Vehicle | q.s. to 100% | q.s. to 100% | q.s. to 100% | q.s. to 100% | q.s. to 100% |

### Example 12

### Determination of Levels of Mecamylamine in the Plasma of Human Subjects Following Ocular Administration of Mecamylamine HCl Ophthalmic Solution

In a phase 1 double-masked, placebo controlled randomized design, healthy volunteers have received mecamylamine HCl ophthalmic solution at concentrations of 0.03%, 0.1%, 0.3%, and 1%. In the first part of this study, subjects received two doses of mecamylamine ophthalmic solution in one eye and placebo in the other eye. Doses were administered as a topical eye drop on a single day with a 6-hour interval between doses. In the second part of the study, subjected received two doses per day of the assigned treatment in both eyes for 14 consecutive days. No blood samples for drug analysis were taken in the first, single-day dosing, part of the study. In the second, 14-day dosing, part of the study, blood samples were taken in subjects on days 1, 7 and 14 of dosing (pre-dose, 1.5 and 3 hours after first dose). A final sample was taken 72 hours after the final dose. Blood samples are being analyzed using a LC-MS/MS method to determine the concentration of mecamylamine.

### Example 13

### 14-Day Dog Toxicology Study for Ocular Administration of Mecamylamine

An ophthalmic saline solution formulation containing 3% mecamylamine hydrochloride (HCl) was evaluated for toxicological effects and drug pharmacokinetics in healthy beagle dogs. The drug solution or a matching vehicle control was administered 2, 4 and 8 times daily for 14 or 15 consecutive days to both eyes of Beagle dogs.

Thirty-two experimentally naïve Beagle dogs (16 males and 16 females), 5-7 months old and weighing 6.0-8.5 kilograms were assigned to treatment groups as shown Table 17 below.

TABLE 17
GLP 14-Day Dog Ocular Toxicity: Treatment Groups

| | | | | Dose Volume | | | |
|---|---|---|---|---|---|---|---|
| | | Dose Level per | | per | Dose Level | Number of | |
| | | administration | Concentration | administration | per day | Animals | |
| Group | Treatment* | (mg/eye/dose) | (mg/ml) | (ml/eye/dose) | (mg/eye/day) | M | F |
| 1 | Vehicle Control | 0 | 0 | 0.050 | 0 | 4 | 4 |
| | (6 doses/day) | (right eye only) | (right eye only) | (right eye only) | (right eye only) | | |
| 2 | MEC 3% | 1.5 | 30 | 0.050 | 3 | 4 | 4 |
| | (2 doses/eye/day) | | | | | | |
| 3 | MEC 3% | 1.5 | 30 | 0.050 | 6 | 4 | 4 |
| | (4 doses/eye/day) | | | | | | |
| 4 | MEC 3% | 1.5 | 30 | 0.050 | 9 | 4 | 4 |
| | (6 doses/eye/day) | | | | | | |

Dosing was as follows: 2/day = q. ˜4 hours, 4/day = q. ˜2 hours, and 6/day = q. ˜1.5 hours for a minimum of 14 days (males)/15 days (females)

Animals were dosed 2, 4 or 6 times a day for 14 consecutive days for males or 15 consecutive days for females. The dose (50 microliters) was administered to the globe of the eye on each dosing occasion. Mortality and clinical observations were evaluated twice daily. Ocular observations were evaluated according to the Draize scoring system twice daily (Draize et al., (1944) J. Pharm. Exp. Ther. 82: 377-390, incorporated by reference herein). All eyes from all animals had ophthalmology examinations using an indirect ophthalmoscope and slit lamp and were evaluated according to McDonald and Shadduck (McDonald & Shadduck (1977) Advances in Modern Toxicology 4: 162 (New York, Wiley), hereby incorporated by reference herein) prior to treatment initiation and 1-2 hours following the final dosing of Groups 1 and 4 on Days 7 and 14. Approximate pupil size was determined for each animal prior to study initiation, at least 30 minutes following the final daily dose on Days 1 and 8 and prior to terminal sacrifice on Days 15/16. Electroretinograms (ERG) were obtained from all animals prior to treatment initiation and on Day 14. Blood samples for evaluation of hematology, coagulation and clinical chemistry parameters were collected prior to treatment initiation and prior to terminal sacrifice on Days 15/16. Selected tissues were harvested at necropsy, selected organs weighed, and selected tissues from all animals were evaluated microscopically.

There were no unscheduled deaths during the study. Additionally, there were no test article-related effects on body weights, food consumption, hematology parameters, coagulation parameters, organ weights, intraocular pressures, or pupil sizes during the study. There were no adverse test article related effects on clinical chemistry parameters.

On most dosing days, except Days 2 and 11, there was at least one Group 4 animal noted with squinting of the left and/or right eye at ˜30 minute post-dose 6. As similar observations were not noted on any days in Group 1 animals, which received the same number of doses of the vehicle to the right eye, the findings were considered related to the six times daily dosing of mecamylamine HCl, 3% ophthalmic solution.

The most common ocular observation by Draize evaluation was a redness score of 1 (of score 3), a definite injection of the vessels of the conjunctivae. The finding was more common at the 1-2 hour post-dosing observation of the Group 3 and/or 4 animals, less frequently in Group 2 animals and rarely observed in Group 1 animals. The sign generally was observed in fewer eyes at the daily pre-dose Draize evaluation, indicating the resolution of the findings from the previous day.

There were no apparent test article related effects observed on Day 7 by McDonald and Shadduck scoring and indirect ophthalmoscopy and slit lamp biomicroscopy. On Day 14, the results of ocular examinations were complicated by, or possibly the direct result of, eye manipulations from the ERG procedure. Results did show conjunctival congestion observed in at least one eye of one Group 1 male, three Group 2 animals, two Group 3 animals, and five of eight Group 4 animals had bilateral conjunctival congestion. Corneal erosions, however, were confined to two Group 4 animals and suggestive of a test article effect.

Examination of all the electroretinographic data, both qualitative and quantitative provided no evidence supporting retinal degeneration or other physiological abnormality attributable to the test compound or vehicle.

There were no test article related gross necropsy findings, and test article related lesions were not apparent from histopathological evaluation of the globes (sections including retina, choroid, sclera, lens, cornea, iris/ciliary body, and optic nerve), eyelid (when present), conjunctiva, extraocular muscle and lacrimal glands (when present) for all animals from the Day 15 and Day 16 sacrifices. Eye sectioning included a central section of approximately 5 mm, which upon evaluation did not reveal any detectable corneal erosions for the two Group 4 animals which were positive for fluorescein staining on Day 14, indicating a plausible resolution of those lesions.

Maximum observed concentrations (Cmax) were highly variable, most notably for the first dosing of Group 2. Cmax was generally linearly dose proportional for Groups 3 and 4 (means of 44 and 64 ng/mL) but Group 2 was well over linearly dose proportional (mean of 49 ng/mL) and had the highest overall value (185 ng/mL) despite being dosed the least. For Group 2, Day 1 values were more variable (relatively) than Day 13; for Groups 3 and 4, Day 1 values were less relatively variable than Day 13 values. The high-dose group (Group 4) had small but measurable mecamylamine values before dosing on Day 13. Except as noted for Group 2, no notable differences were found in toxicokinetics between Day 1 and Day 13. The accumulation ratios were a maximum of 1.14, indicating no significant accumulation for any group after 13 days of ophthalmological dosing. There was also no indication of significant induction or inhibition of metabolism of the drug.

In conclusion, a 3% mecamylamine hydrochloride, 3% ophthalmic solution was not associated with any definitive adverse effects upon twice daily dosing (˜4 hours between doses, totaling 3 mg/eye/day) or four times daily dosing (˜2 hours between doses, totaling 6 mg/eye/day) for fourteen (males) or fifteen (females) consecutive days to Beagle dogs. Beagle dogs administered six times daily dosing (˜1.5 hours between doses, totaling 9 mg/eye/day) of mecamylamine HCl, 3% ophthalmic solution had clinical signs of squinting (not present in controls) on several days, the most consistent Draize scores, although mild, and the appearance of corneal erosions on Day 14 in two of eight dogs may have been suggestive of a test article effect. Therefore, the no observable adverse effect level (NOAEL) of topical mecamylamine HCl, 3% ophthalmic solution is considered to be four times daily dosing (˜2 hours between doses) totaling 6 mg/eye/day for fifteen consecutive days.

### Example 14

### A 39-Week Ocular Toxicity Study of Mecamylamine Hydrochloride Ophthalmic Solution in Dogs with a 4-Week Recovery

The purpose of this study is to characterize the general and ocular toxicity and toxicokinetics of the test article when administered to healthy Beagle dogs, twice daily by topical ocular application for thirty-nine weeks followed by a four-week recovery period and including a 13-week interim sacrifice.

The conduct of a chronic toxicity study in a nonrodent species is required prior to longer-term human use. The Beagle dog is a standard non-rodent species used in toxicology studies based upon the substantial amounts of published historical data. The number of animals used in this study is required to define the toxicity, and reversibility thereof, and toxicokinetics of the test article with longer-term repeated dosing.

Three treatment groups were included in this study as described in the following table.

TABLE 18
Group Assignments and Dose Levels

| | Dose Level per | Dose Volume per | | Dose Level | Number of | |
|---|---|---|---|---|---|---|
| | administration | administration | Concentration | per day | Animals | |
| Group | (mg/eye/dose) | (ml/eye) | (mg/ml) | (mg/eye/day) | Male | Female |
| 1. Placebo Solution | 0 | 0.050 | 0 | 0 | 10 | 10 |
| 2. 1% Mecamylamine | 0.5 | 0.050 | 10 | 1 | 7 | 7 |
| 3. 3% Mecamylamine | 1.5 | 0.050 | 30 | 3 | 10 | 10 |

Study drug is being administered by ocular instillation twice daily (approximately 6 hours between doses.) The ocular route was chosen, as it is the intended route of administration in humans.

Following 13 weeks of dosing, 3 animals/sex in Groups 1, 2 and 3 will be euthanized by barbiturate overdose and necropsied. Following 39 weeks of dosing, 4 animals/sex/group will be euthanized by barbiturate overdose and necropsied. Following the treatment phase, 3 animals/sex in Groups 1 and 3 will remain on study, untreated, and be euthanized by barbiturate overdose and necropsied following a four-week recovery period.

During the study animals are being observed for mortality and clinical observations at least once a day. Ocular observations according to Draize are being recorded twice weekly with the first recorded observations occurring on Day 1. The observations are being recorded prior to the first dose on a particular day and approximately 1-2 hours following the second dose on the same day. Ocular scoring is being recorded once prior to scheduled sacrifices following 13 weeks and 39 weeks of dosing and following the four-week recovery period.

After approximately 1 month of drug administration there have been no clinically significant observations related to the possible toxicology of the test article.

What is claimed as new and desired to be protected by Letters Patent of the United States is:

1. A method for treating or preventing conditions mediated by neovascularization, abnormal angiogenesis, vascular permeability, or combinations thereof, of posterior tissues, anterior tissues or fluid of the eye comprising the step of
a) topically applying to one or both eyes of an individual in need thereof a formulation comprising mecamylamine, or a pharmaceutically acceptable salt thereof, and a carrier suitable for topical administration to the eye,
wherein the mecamylamine or a pharmaceutically acceptable salt thereof is present in the formulation in an amount sufficient to deliver a therapeutically effective amount of mecamylamine to one or more of the posterior or anterior tissues or fluids of the eye for the treatment or prevention of conditions mediated by neovascularization, abnormal angiogenesis, vascular permeability, or combinations thereof, of posterior tissues, anterior tissues or fluids of the eye.

2. A method for treating or preventing conditions mediated by neovascularization, abnormal angiogenesis, vascular permeability, or combinations thereof, of posterior tissues of the eye comprising the step of
a) topically applying to one or both eyes of an individual in need thereof a formulation comprising mecamylamine, or a pharmaceutically acceptable salt thereof, and a carrier suitable for topical administration to the eye,
wherein the mecamylamine or a pharmaceutically acceptable salt thereof is present in the formulation in an amount sufficient to deliver a therapeutically effective amount of mecamylamine to one or more of the posterior tissues of the eye for the treatment or prevention of conditions mediated by neovascularization, abnormal angiogenesis, vascular permeability, or combinations thereof, of posterior tissues of the eye.

3. The method of claim 2, wherein when the formulation is topically administered to a rabbit eye, the ratio of the concentration of mecamylamine present in choroidal and retinal tissue, measured in units of ng/g, to the concentration of mecamylamine in plasma measured in units of ng/mL ([ng/g mecamylamine choroidal+retinal tissue]: [ng/mL plasma]) is at least about 40:1.

4. The method of claim 1, wherein when the formulation is topically administered to a rabbit eye, the ratio of the concentration of mecamylamine present in choroidal and retinal tissue, measured in units of ng/g, to the concentration of mecamylamine in plasma measured in units of ng/mL ([ng/g mecamylamine choroidal+retinal tissue]: [ng/mL plasma]) is at least about 20:1.

5. The method of claim 3, wherein the ratio is at least about 80:1

6. The method of claim 3, wherein the ratio is at least about 300:1.

7. The method of claim 3, wherein the ratio is from about 40:1 to about 1000:1.

8. The method of claim 3, wherein the ratio is from about 40:1 to about 2000:1.

9. The method of claim 4, wherein the ratio is from about 20:1 to about 1000:1.

10. The method of claim 3, wherein the ratio is from about 40:1 to about 2000:1.

11. The method of claim 1, wherein when the formulation is topically administered to a rabbit eye, the mean maximum concentration of mecamylamine in plasma is less than about 70 ng/mL.

12. The method of claim 11, wherein the mean maximum concentration of mecamylamine in plasma is less than about 50 ng/mL.

13. The method of claim 11, wherein the mean maximum concentration of mecamylamine in plasma is less than about 25 ng/mL.

14. The method of claim 11, wherein the mean maximum concentration of mecamylamine in plasma is less than about 10 ng/mL.

15. The method of claim 11, wherein the mean maximum concentration of mecamylamine in plasma is less than about 5 ng/mL.

16. The method of claim 1, wherein when the formulation is topically administered to a rabbit eye, the total concentration of mecamylamine in plasma measured as the area under the curve is less than about 100 ng/mL-hr.

17. The method of claim 2, wherein when the formulation is topically administered to a rabbit eye, the total concentration of mecamylamine in plasma measured as the area under the curve is less than about 100 ng/mL-hr.

18. The method of claim 1, wherein the mecamylamine is incorporated in the formulation as substantially pure S-mecamylamine.

19. The method of claim 1, wherein the mecamylamine is incorporated in the formulation as substantially pure R-mecamylamine.

20. The method of claim 2, wherein the mecamylamine is incorporated in the formulation as substantially pure S-mecamylamine.

21. The method of claim 2, wherein the mecamylamine is incorporated in the formulation as substantially pure R-mecamylamine.

22. The method of claim 2, wherein the carrier comprises an aqueous saline solution.

23. The method of claim 22, wherein the aqueous saline solution is isotonic.

24. The method of claim 1, wherein the carrier comprises water.

25. The method of claim 24, wherein the formulation is substantially free of surfactant.

26. The method of claim 24, wherein the formulation further comprises one or more of a preservative or a surfactant.

27. The method of claim 26, wherein the formulation comprises a preservative.

28. The method of claim 27, wherein the preservative is selected from the group consisting of benzalkonium chloride, benzethonium chloride, chlorhexidine, chlorobutanol, methylparaben, phenylethyl alcohol, propylparaben, thimerosal, phenylmercuric nitrate, phenylmercuric borate, and phenylmercuric acetate.

29. The method of claim 28, wherein the preservative is benzalkonium chloride.

30. The method of claim 24, wherein the carrier further comprises one or more tonicity agent(s).

31. The method of claim 30, wherein the one or more tonicity agent(s) is a polyol.

32. The method of claim 31, wherein the polyol is a sugar alcohol, trihydroxy alcohol, propylene glycol or polyethylene glycol.

33. The method of claim 30, where the one or more tonicity agent(s) is mannitol, glycerin or a combination thereof.

34. The method of claim 24, wherein when the formulation is topically administered to a rabbit eye, the mean maximum concentration of mecamylamine in plasma is less than about 70 ng/mL.

35. The method of claim 34, wherein the mean maximum concentration of mecamylamine in plasma is less than about 50 ng/mL.

36. The method of claim 34, wherein the mean maximum concentration of mecamylamine in plasma is less than about 25 ng/mL.

37. The method of claim 34, wherein the mean maximum concentration of mecamylamine in plasma is less than about 10 ng/mL.

38. The method of claim 34, wherein the mean maximum concentration of mecamylamine in plasma is less than about 5 ng/mL.

39. The method of claim 24, wherein the formulation further comprises a chelating agent.

40. The method of claim 24, wherein the formulation is substantially free of polymer.

41. The method of claim 40, wherein the formulation is isotonic.

42. The method of claim 40, wherein the formulation is substantially free of surfactant.

43. The method of claim 40, wherein the formulation further comprises one or more of a preservative or a surfactant.

44. The method of claim 43, wherein the formulation comprises a preservative.

45. The method of claim 44, wherein the preservative is selected from the group consisting of benzalkonium chloride, benzethonium chloride, chlorhexidine, chlorobutanol, methylparaben, phenylethyl alcohol, propylparaben, thimerosal, phenylmercuric nitrate, phenylmercuric borate, and phenylmercuric acetate.

46. The method of claim 45, wherein the preservative is benzalkonium chloride.

47. The method of claim 40, wherein when the formulation is topically administered to a rabbit eye, the mean maximum concentration of mecamylamine in plasma is less than about 70 ng/mL.

48. The method of claim 47, wherein the mean maximum concentration of mecamylamine in plasma is less than about 50 ng/mL.

49. The method of claim 47, wherein the mean maximum concentration of mecamylamine in plasma is less than about 25 ng/mL.

50. The method of claim 47, wherein the mean maximum concentration of mecamylamine in plasma is less than about 10 ng/mL.

51. The method of claim 47, wherein the mean maximum concentration of mecamylamine in plasma is less than about 5 ng/mL.

52. The method of claim 40, wherein the formulation further comprises a chelating agent.

53. The method of claim 24, wherein the carrier further comprises a viscosity-increasing agent.

54. The method of claim 53, wherein the viscosity-increasing agent is selected from the group consisting of water soluble cellulose derivatives, polyvinyl alcohol, polyvinyl pyrrolidone, chondroitin sulfate, hyaluronic acid, and soluble starches.

55. The method of claim 54, wherein the viscosity-increasing agent is a water soluble cellulose derivative.

56. The method of claim 55, wherein the viscosity-increasing agent is hypromellose.

57. The method of claim 53, wherein the mecamylamine is incorporated in the formulation as substantially pure S-mecamylamine.

58. The method of claim 53, wherein the mecamylamine is incorporated in the formulation as substantially pure R-mecamylamine.

59. The method of claim 53, wherein the carrier further comprises one or more tonicity agent(s).

60. The method of claim 59, wherein the one or more tonicity agent(s) is a polyol.

61. The method of claim 60, wherein the polyol is a sugar alcohol, trihydroxy alcohol, propylene glycol or polyethylene glycol.

62. The method of claim 59, where the one or more tonicity agent(s) is mannitol, glycerin or a combination thereof.

63. The method of claim 53, wherein the formulation is substantially free of surfactant.

64. The method of claim 53, wherein the formulation further comprises one or more of a preservative or a surfactant.

65. The method of claim 64, wherein the formulation comprises a preservative.

66. The method of claim 65, wherein the preservative is selected from the group consisting of benzalkonium chloride, benzethonium chloride, chlorhexidine, chlorobutanol, methylparaben, phenylethyl alcohol, propylparaben, thimerosal, phenylmercuric nitrate, phenylmercuric borate, and phenylmercuric acetate.

67. The method of claim 66, wherein the preservative is benzalkonium chloride.

68. The method of claim 53, wherein when the formulation is topically administered to a rabbit eye, the mean maximum concentration of mecamylamine in plasma measured in units of ng/mL is less than about 70 ng/mL.

69. The method of claim 68, wherein the mean maximum concentration of mecamylamine in plasma measured in units of ng/mL is less than about 50 ng/mL.

70. The method of claim 68, wherein the mean maximum concentration of mecamylamine in plasma is less than about 25 ng/mL.

71. The method of claim 68, wherein the mean maximum concentration of mecamylamine in plasma is less than about 10 ng/mL.

72. The method of claim 68, wherein the mean maximum concentration of mecamylamine in plasma is less than about 5 ng/mL.

73. The method of claim 1, wherein the formulation comprises from about 0.001% to about 6% (w/v) mecamylamine or a pharmaceutically acceptable salt thereof.

74. The method of claim 73, wherein the formulation comprises from about 0.001% to about 3% (w/v) mecamylamine or a pharmaceutically acceptable salt thereof.

75. The method of claim 74, wherein the formulation comprises from about 0.03% to about 3% (w/v) mecamylamine or a pharmaceutically acceptable salt thereof.

76. The method of claim 75, wherein the formulation comprises from about 0.1% to about 1% (w/v) mecamylamine or a pharmaceutically acceptable salt thereof.

77. The method of claim 1, wherein the carrier comprises an aqueous isotonic solution and wherein the formulation further comprises a chelating agent and a preservative.

78. The method of claim 1, wherein the formulation further comprises one or more buffering agents.

79. The method of claim 78, wherein the one or more buffering agent(s) is selected from the group consisting of phosphate buffers, citrate buffers, maleate buffers, borate buffers and combinations thereof.

80. The method of claim 79, wherein the carrier further comprises a viscosity-increasing agent.

81. The method of claim 80, wherein the formulation comprises from about 0.001% to about 6% (w/v) mecamylamine or a pharmaceutically acceptable salt thereof.

82. The method of claim 81, wherein the formulation comprises from about 0.001% to about 3% (w/v) mecamylamine or a pharmaceutically acceptable salt thereof.

83. The method of claim 82, wherein the formulation comprises from about 0.03% to about 3% (w/v) mecamylamine or a pharmaceutically acceptable salt thereof.

84. The method of claim 83, wherein the formulation comprises from about 0.1% to about 1% (w/v) mecamylamine or a pharmaceutically acceptable salt thereof.

85. The method of claim 84, wherein the viscosity-increasing agent is hypromellose.

86. The method of claim 2, wherein the carrier comprises from about 0.03% to about 2% (w/v) of a gel-forming polymer and water,
wherein the gel-forming polymer is selected such that when the formulation is topically administered to a rabbit eye the ratio of the concentration of mecamylamine present in choroidal and retinal tissue, measured in units of ng/g, to the concentration of mecamylamine in plasma, measured in units of ng/mL, ([ng/g mecamylamine choroidal+retinal tissue]: [ng/mL plasma]) is at least about 300:1.

87. The method of claim 86, wherein when the formulation is topically administered to a rabbit eye, the mean maximum concentration of mecamylamine in plasma is less than about 70 ng/mL.

88. The method of claim 87, wherein the mean maximum concentration of mecamylamine in plasma is less than about 50 ng/mL.

89. The method of claim 87, wherein the mean maximum concentration of mecamylamine in plasma is less than about 25 ng/mL.

90. The method of claim 87, wherein the mean maximum concentration of mecamylamine in plasma is less than about 10 ng/mL.

91. The method of claim 87, wherein the mean maximum concentration of mecamylamine in plasma is less than about 5 ng/mL.

92. The method of claim 86, wherein the mecamylamine is incorporated in the formulation as substantially pure S-mecamylamine.

93. The method of claim 86, wherein the mecamylamine is incorporated in the formulation as substantially pure R-mecamylamine.

94. The method of claim 86, wherein the formulation is a gel prior to topical ocular administration.

95. The method of claim 86, wherein the formulation forms a gel in situ upon topical ocular administration.

96. The method of claim 86, wherein the gel-forming polymer is a polysaccharide.

97. The method of claim 86, wherein the carrier further comprises one or more tonicity agent(s).

98. The method of claim 97, wherein the one or more tonicity agent(s) is a polyol.

99. The method of claim 98, wherein the polyol is a sugar alcohol, trihydroxy alcohol, propylene glycol or polyethylene glycol.

100. The method of claim 97, where the one or more tonicity agent(s) is mannitol, glycerin or a combination thereof.

101. The method of claim 96, wherein the polysaccharide is gellan gum.

102. The method of claim 2, wherein the carrier comprises from about 0.05% to about 2% (w/v) gellan gum.

103. The method of claim 102, wherein the carrier comprises from about 0.1% to about 1% (w/v) gellan gum.

104. The method of claim 103, wherein the carrier comprises from about 0.1% to about 0.6% (w/v) gellan gum.

105. The method of claim 2, wherein the formulation is substantially free of surfactant.

106. The method of claim 2, wherein the formulation further comprises one or more of a preservative or a surfactant.

107. The method of claim 106, wherein the formulation comprises a preservative.

108. The method of claim 107, wherein the preservative is selected from the group consisting of benzalkonium chloride, benzethonium chloride, chlorhexidine, chlorobutanol, methylparaben, phenylethyl alcohol, propylparaben, thimerosal, phenylmercuric nitrate, phenylmercuric borate, and phenylmercuric acetate.

109. The method of claim 108, wherein the preservative is benzalkonium chloride.

110. The method of claim 1 wherein the formulation further comprises a chelating agent.

111. The method of claim 110, wherein the chelating agent is edetate disodium (dihydrate).

112. The method of claim 1, wherein the carrier further comprises one or more tonicity agent(s).

113. The method of claim 1112, wherein the one or more tonicity agent(s) is a polyol.

114. The method of claim 1113, wherein the polyol is a sugar alcohol, trihydroxy alcohol, propylene glycol or polyethylene glycol.

115. The method of claim 112, where the one or more tonicity agent(s) is mannitol, glycerin or a combination thereof.

116. The method of claim 2, wherein the formulation comprises from about 0.001% to about 6% (w/v) mecamylamine or a pharmaceutically acceptable salt thereof.

117. The method of claim 116, wherein the formulation comprises from about 0.001% to about 5% (w/v) mecamylamine or a pharmaceutically acceptable salt thereof.

118. The method of claim 117, wherein the formulation comprises from about 0.03% to about 4% (w/v) mecamylamine or a pharmaceutically acceptable salt thereof.

119. The method of claim 118, wherein the formulation comprises from about 0.03% to about 3% (w/v) mecamylamine or a pharmaceutically acceptable salt thereof.

120. The method of claim 119, wherein the formulation comprises from about 0.03% to about 2% (w/v) mecamylamine or a pharmaceutically acceptable salt thereof.

121. The method of claim 120, wherein the formulation comprises from about 0.1% to about 1% (w/v) mecamylamine or a pharmaceutically acceptable salt thereof.

122. The method of claim 121, wherein the carrier comprises from about 0.05% to about 1% (w/v) gellan gum and water.

123. The method of claim 2, wherein the individual has been identified as having one or more conditions mediated by retinal neovascularization, choroidal neovascularization, abnormal angiogenesis, vascular permeability, or combinations thereof, of posterior tissues of the eye.

124. The method of claim 2, wherein the individual has been identified as susceptible to one or more conditions mediated by retinal neovascularization, choroidal neovascularization, abnormal angiogenesis, vascular permeability, or combinations thereof, of posterior tissues of the eye.

125. The method of claim 2, wherein the individual has been identified as having or being susceptible to a proliferative retinopathy.

126. The method of claim 123, wherein the individual has been identified as having or being susceptible to a non-neovascular form of macular degeneration.

127. The method of claim 2, wherein the condition is diabetic retinopathy, retinopathy of prematurity, retinal neovascularization associated with macular degeneration, choroidal neovascularization associated with macular degeneration, retinopathy associated with macular edema, or retinopathy associated with sickle cell disease.

128. The method of claim 2, wherein the condition is diabetic retinopathy.

129. The method of claim 2, wherein the condition is retinopathy of prematurity.

130. The method of claim 2, wherein the condition is retinal neovascularization or choroidal neovascularization associated with macular degeneration.

131. The method of claim 130, wherein the condition is an age-related maculopathy.

132. The method of claim 130, wherein the condition is age-related macular degeneration.

133. The method of claim 132, wherein the age-related macular degeneration is a neovascular form of age-related macular degeneration.

134. The method of claim 1, wherein the condition is associated with abnormal angiogenesis affecting the anterior tissues of the eye or is a condition involving abnormal angiogenesis affecting both anterior and posterior tissues of the eye.

135. The method of claim 134, wherein the condition is associated with abnormal angiogenesis affecting the anterior tissues of the eye.

136. The method of claim 135, wherein the condition is corneal neovascularization, pterygium, post-corneal transplant neovascularization, rubeosis iridis, or neovascular glaucoma.

137. The method of claim 1, wherein the condition involves vitreal, retinal or choroidal neovascularization.

138. The method of claim 134, wherein the condition is an ocular tumor.

139. The method of claim 1, wherein the individual is a mammal.

140. The method of claim 139, wherein the mammal is a primate, rabbit, canine, feline, or rodent.

141. The method of claim 140, wherein the mammal is a primate.

142. The method of claim 141, wherein the primate is a human.

143. The method of claim 2, wherein the individual is a mammal.

144. The method of claim 143, wherein the mammal is a primate, rabbit, canine, feline, or rodent.

145. The method of claim 144, wherein the mammal is a primate.

146. The method of claim 145, wherein the primate is a human.

147. The method of claim 40, wherein the individual is a human.

148. The method of claim 2, wherein the therapeutically effective amount of mecamylamine is delivered to the retina.

149. The method of claim 2, wherein the therapeutically effective amount of mecamylamine is delivered to the choroid.

150. The method of claim 2, wherein the therapeutically effective amount of mecamylamine is delivered to the retina and the choroid.

151. The method of claim 1, wherein step (a) is performed once per day, twice per day, three times per day, four times per day, once every other day, once per week, or twice per week.

152. The method of claim 1, further comprising a step (b), where step (b) comprises
administering to the individual a pharmaceutical agent, additional treatment modality or combination thereof.

153. The method of claim 152, wherein step (b) is performed prior to or concomitantly with step (a).

154. The method of claim 152, wherein the pharmaceutical agent is a VEGF antagonist, tyrosine kinase inhibitor or VEGF scavenger.

155. The method of claim 154, wherein the VEGF antagonist is a VEGF aptamer.

156. The method of claim 155, wherein the VEGF aptamer is pegaptanib.

157. The method of claim 152, wherein the pharmaceutical agent is an anti-VEGF antibody or fragment thereof.

158. The method of claim 157, wherein the anti-VEGF antibody is bevacizumab, ranibizumab, or a combination thereof.

159. The method of claim 152, wherein, the additional treatment modality is thermal laser photocoagulation or photodynamic therapy.

160. The method of claim 2, wherein step (a) is performed once per day, twice per day, three times per day, four times per day, once every other day, once per week, or twice per week.

161. The method of claim 2, further comprising a step (b), where step (b) comprises
administering to the individual a pharmaceutical agent, additional treatment modality or combination thereof.

162. The method of claim 161, wherein step (b) is performed prior to or concomitantly with step (a).

163. The method of claim 161, wherein the pharmaceutical agent is a VEGF antagonist, VEGF scavenger or tyrosine kinase inhibitor.

164. The method of claim 163, wherein the VEGF antagonist is a VEGF aptamer.

165. The method of claim 164, wherein the VEGF aptamer is pegaptanib.

166. The method of claim 161, wherein the pharmaceutical agent is an anti-VEGF antibody or fragment thereof.

167. The method of claim 166, wherein the anti-VEGF antibody is bevacizumab, ranibizumab, or a combination thereof.

168. The method of claim 161, wherein, the additional treatment modality is thermal laser photocoagulation or photodynamic therapy.

169. A method for treating or preventing conditions mediated by neovascularization, abnormal angiogenesis, vascular permeability, or combinations thereof, of anterior tissues of the eye comprising the step of
a) topically applying to one or both eyes of an individual in need thereof a formulation comprising mecamylamine, or a pharmaceutically acceptable salt thereof, and a carrier suitable for topical administration to the eye,
wherein the mecamylamine or a pharmaceutically acceptable salt thereof is present in the formulation in an amount sufficient to deliver a therapeutically effective amount of mecamylamine to one or more of the anterior tissues or fluids of the eye for the treatment or prevention of conditions mediated by neovascularization, abnormal angiogenesis, vascular permeability, or combinations thereof, of anterior tissues of the eye.

170. The method of claim 169, wherein when the formulation is topically administered to a rabbit eye, the ratio of the concentration of mecamylamine present in corneal tissue, measured in units of ng/g, to the concentration of mecamylamine in plasma measured in units of ng/mL ([ng/g mecamylamine corneal tissue]: [ng/mL plasma]) is at least about 100:1.

171. The method of claim 170, wherein the ratio is at least about 1000:1

172. The method of claim 171, wherein the ratio is at least about 1500:1.

173. The method of claim 172, wherein the ratio is from about 1000:1 to about 4000:1.

174. The method of claim 173, wherein the ratio is from about 1000:1 to about 3000:1.

175. The method of claim 169, wherein when the formulation is topically administered to a rabbit eye, the mean maximum concentration of mecamylamine in plasma is less than about 70 ng/mL.

176. The method of claim 175, wherein the mean maximum concentration of mecamylamine in plasma is less than about 50 ng/mL.

177. The method of claim 175, wherein the mean maximum concentration of mecamylamine in plasma is less than about 25 ng/mL.

178. The method of claim 175, wherein the mean maximum concentration of mecamylamine in plasma is less than about 10 ng/mL.

179. The method of claim 175, wherein the mean maximum concentration of mecamylamine in plasma is less than about 5 ng/mL.

180. The method of claim 169, wherein the mecamylamine is incorporated in the formulation as substantially pure S-mecamylamine.

181. The method of claim 169, wherein the mecamylamine is incorporated in the formulation as substantially pure R-mecamylamine.

182. The method of claim 169, wherein the therapeutically effective amount of mecamylamine is delivered to the cornea, iris, sclera, trabecular meshwork or lens.

183. The method of claim 169, wherein the therapeutically effective amount of mecamylamine is delivered to the cornea.

184. The method of claim 169, wherein the therapeutically effective amount of mecamylamine is delivered to the lens.

185. The method of claim 169, wherein the therapeutically effective amount of mecamylamine is delivered to the iris.

186. The method of claim 169, wherein the therapeutically effective amount of mecamylamine is delivered to the sclera.

187. The method of claim 169, wherein the therapeutically effective amount of mecamylamine is delivered to the trabecular meshwork.

188. The method of claim 169, wherein the condition is associated with abnormal angiogenesis affecting the anterior tissues of the eye or is a condition involving abnormal angiogenesis affecting both the anterior and posterior tissues of the eye.

189. The method of claim 169, wherein the condition is corneal neovascularization, pterygium, post-corneal transplant neovascularization, rubeosis iridis, or neovascular glaucoma.

190. The method of claim 188, wherein the condition is an ocular tumor.

191. The method of claim 169, wherein step (a) is performed once per day, twice per day, three times per day, four times per day, once every other day, once per week, or twice per week.

192. The method of claim 169, further comprising a step (b), where step (b) comprises administering to the individual a pharmaceutical agent, additional treatment modality or combination thereof.

193. The method of claim 192, wherein step (b) is performed prior to or concomitantly with step (a).

194. A pharmaceutical formulation comprising mecamylamine, or a pharmaceutically acceptable salt thereof, water and a gel-forming polymer formulated for ocular topical administration,
wherein the gel-forming polymer is selected such that when the formulation is topically administered to a rabbit eye, the ratio of the concentration of mecamylamine present in the choroidal and retinal tissue, measured in units of ng/g, to the concentration of mecamylamine in plasma measured in units of ng/mL ([ng/g mecamylamine choroidal+retinal tissue]: [ng/mL plasma]) is at least about 300:1.

195. The formulation of claim 194, wherein the ratio is from about 300:1 to about 1000:1

196. The formulation of claim 194, wherein the ratio is at least about 350:1.

197. The formulation of claim 194, wherein when the formulation is topically administered to a rabbit eye, the mean maximum concentration of mecamylamine in plasma is less than about 70 ng/mL.

198. The formulation of claim 197, wherein the mean maximum concentration of mecamylamine in plasma is less than about 50 ng/mL.

199. The formulation of claim 198, wherein the mean maximum concentration of mecamylamine in plasma is less than about 25 ng/mL.

200. The formulation of claim 198, wherein the mean maximum concentration of mecamylamine in plasma is less than about 10 ng/mL.

201. The formulation of claim 198, wherein the mean maximum concentration of mecamylamine in plasma is less than about 5 ng/mL.

202. The formulation of claim 194, wherein the gel-forming polymer is present at a concentration of from about 0.03% to about 2% (w/v).

203. The formulation of claim 194, wherein the formulation is a gel prior to topical ocular administration.

204. The formulation of claim 194, wherein the formulation forms a gel in situ upon topical ocular administration.

205. The formulation of claim 194, wherein the gel-forming polymer is a polysaccharide.

206. The formulation of claim 205, wherein the polysaccharide is gellan gum.

207. The formulation of claim 205, wherein the gel-forming polymer is gellan gum present at a concentration of from about 0.05% to about 2% (w/v).

208. The formulation of claim 206, wherein the gellan gum is present at a concentration of about 0.1% to about 1% (w/v).

209. The formulation of claim 208, wherein the gellan gum is present at a concentration of about 0.1% to about 0.6% (w/v).

210. The formulation of claim 194, wherein the carrier further comprises one or more tonicity agent(s).

211. The formulation of claim 210, wherein the one or more tonicity agent(s) is a polyol.

212. The formulation of claim 211, wherein the polyol is a sugar alcohol, trihydroxy alcohol, propylene glycol or polyethylene glycol.

213. The formulation of claim 210, where the one or more tonicity agent(s) is mannitol, glycerin or a combination thereof.

214. The formulation of claim 194, wherein the formulation is substantially free of surfactant.

215. The formulation of claim 194, further comprising one or more of a preservative or a surfactant.

216. The formulation of claim 194, wherein the formulation comprises a preservative.

217. The formulation of claim 216, wherein the preservative is selected from the group consisting of benzalkonium chloride, benzethonium chloride, chlorhexidine, chlorobutanol, methylparaben, phenylethyl alcohol, propylparaben, thimerosal, phenylmercuric nitrate, phenylmercuric borate, and phenylmercuric acetate.

218. The formulation of claim 217, wherein the preservative is benzalkonium chloride.

219. The formulation of claim 218, wherein the mecamylamine or a pharmaceutically acceptable salt thereof is present at concentration of from about 0.001% to about 6% (w/vol).

220. The formulation of claim 219, wherein the mecamylamine or a pharmaceutically acceptable salt thereof is present at concentration of from about 0.001% to about 5% (w/vol).

221. The formulation of claim 220, wherein the formulation comprises from about 0.03% to about 4% (w/v) mecamylamine or a pharmaceutically acceptable salt thereof.

222. The formulation of claim 221, wherein the formulation comprises from about 0.03% to about 3% (w/v) mecamylamine or a pharmaceutically acceptable salt thereof.

223. The formulation of claim 222, wherein the formulation comprises from about 0.03% to about 2% (w/v) mecamylamine or a pharmaceutically acceptable salt thereof.

224. The formulation of claim 223, wherein the formulation comprises from about 0.1% to about 1% (w/v) mecamylamine or a pharmaceutically acceptable salt thereof.

225. The formulation of claim 219, wherein the gel-forming polymer is gellan gum present at a concentration of from about 0.05% to about 1% (w/v).

226. The formulation of claim 194, wherein the formulation comprises a pharmaceutically acceptable salt of mecamylamine.

227. The formulation of claim 226, wherein the salt of mecamylamine is mecamylamine hydrochloride.

228. The formulation of claim 194, wherein the mecamylamine is incorporated in the formulation as substantially pure S-mecamylamine.

229. The formulation of claim 194, wherein the mecamylamine is incorporated in the formulation as substantially pure R-mecamylamine.

230. The formulation of claim 194, further comprising a pharmaceutical agent.

231. A kit comprising a formulation of claim 194, packaging and instructions for use.

232. The kit of claim 231, wherein the formulation is provided in a multi-dose form.

233. The kit of claim 231, wherein the formulation is provided in one or more single unit dose forms.

234. The kit of claim 231, wherein sufficient formulation is provided for treatment over a period of about 1 day, about 1 week, about 2 weeks, about 3 weeks, about 4 weeks, about 1 month, about 2 months, about 3 months, about 4 months, about 6 months, about 9 months or about 1 year.

235. The kit of claim 231, further comprising one or more non-mecamylamine nicotinic acetylcholine receptor antagonists.

236. The kit of claim 231, further comprising one or more pharmaceutical agents.

237. The kit of claim 231, wherein said pharmaceutical agent is provided in a separate container from the pharmaceutical formulation of mecamylamine, or a pharmaceutically acceptable salt thereof.

238. The kit of claim 231, wherein the mecamylamine is incorporated in the formulation as substantially pure S-mecamylamine.

239. The kit of claim 231, wherein the mecamylamine is incorporated in the formulation as substantially pure R-mecamylamine.

240. A method for the preparation of the formulation of claim 194, comprising the steps of
(a) dispersing a gel-forming polymer in an aqueous solution of mecamylamine, or a pharmaceutically acceptable salt thereof;
(b) mixing the mixture formed in step (a) to form a solution or gel; and,

241. The method of claim 240, further comprising the step of
(c) equilibrating the solution or gel formed in step (b).

242. The method according to claim 240, wherein the aqueous solution of mecamylamine, or a pharmaceutically acceptable salt thereof, further comprises a pharmaceutical agent, a preservative or a surfactant.

243. The method according to claim 240, wherein the aqueous solution of mecamylamine, or a pharmaceutically acceptable salt thereof, further comprises a preservative.

244. The method according to claim 243, wherein the aqueous solution of mecamylamine, or a pharmaceutically acceptable salt thereof, comprises a surfactant.

245. The method according to claim 243, wherein the solution formed in step (c) forms a gel in situ upon topical ocular administration.

246. The method of claim 240, wherein the solution formed in step (b) or step (c) is a gel prior to topical ocular administration.

247. The method of claim 240, wherein the mixing in step (b) comprises stirring, heating or a combination thereof.

248. The method of claim 24, wherein the formulation further comprises a chelating agent, one or more preservatives, and one or more tonicity agents.

249. The method of claim 248, where wherein the formulation further comprises one or more buffering agents.

250. The method of claim 40, wherein the formulation further comprises a chelating agent, one or more preservatives, and one or more tonicity agents.

251. The method of claim 250, where wherein the formulation further comprises one or more buffering agents.
